SEQUENCE LISTING (51 sequences)

SEQ ID NO: 1 - DNA, 32146 bp, Homo sapiens
  source 1..32146: mol_type = unassigned DNA
aggcgcggacgcaggttacagcagcgcttggcctctgctgatgccgtcgttatcctacccctcccccgtcccagctctacggcggccgcgcgctccaggccggtcgctccaccccccggctcccgggactgtggactccacgaccctgtcctcggccctgtccgcgccgaagcagcccgggactgcgcagcgccccgcgtgccggtgagcgaccccggacgtgcagtccccaggtccccttccggagcgaggctgagcggctgcgccgcggcttgagccgggcctgacaaggaggggaggccgctggggtgctgggccagccgcaggtacggcggcggtcagggctggtgatcgagttgctgtgtcgcctctaatgaggcccagccagggaacactcggcttcggcccaagccctacctctgggttgcctctaggccctcacaccttgagcgccaggtggccctcttccttttgctgttgatgaatgttcttgccgtggtgccgggatcttattttgcagctttacagagtaggctttgtgctaggtgttgtttatggagcttttcaaaggcgcttgtttccttagttctcaagcccttttagaactaaaaccagtgtctacacagaagatcaggagccatttaaattggagtgctgtgatatattttttctctccaaactgagtctgatatccatgttgactttcagtaaactaaggaaccgctataataaataacactgagcccacagaggttgcagaagagggtctagcaccccttttttaaaccattgagtctcccaagaataaaagtggttacacttgtcagttctctaaggaaaatctagtagagaaggcaagggattcccagtttctcacagagtctggcacttgtccaaacacctaccctaaagcagaatcgatcatggcagctattagattaggacagaaaacaggctcaaacggagagccacttgagtctgcctttctgtgtcttttttctcaacgcgctccagttaagatgcagcttacagcctggctgtgttgactgagatcccagaaagagtgcccttaaggccttgctgataaggcccaatgatcccaattcatctagtgttccgcatagaccccaaggttacagtgtgtggggacagtgatgttaagtcttccctatcctacctgtggctgacaggtgttgattaagacaatgattttttagaagtcataaatcatttgtatttttgatgtgggcagtgatttttataaatacatagttttttttttttgaaagagtgaaaatctcattttacatttgtttgcataccatttgtatagttttttaaattctgtttatttgagtgacagcaacaagtggatggcatgcgaattttatttatttttttatctttgtatttattttgtgtatattcatggagtacaagtgccatttgctacattgatacacaacatcctggtgaagtcagagcccttagtgcatccagcactggagcaacacacattgtacccaccaagcaggctcctatcaggcatgccagttctagaaagtaaaattttcttcatactctaggatgaactgacttacttggaatgtctgttttgtttaatcaaataagtgagaatttttgaaagaaatactatctgtctgaatgtatgtaatctatttttaggaaccgctaaaagacatatcaaatttagaaggagtataattatgttatttttcagttttactttttcactttattttttctcaggatataagcaggaaaataccagcagggtgtattatatgactgttaagttcttttcagctttaaaagggtatgaccttaagtataaagttacttcattatctacctgcagaagttatttttgtgtaggcactagtactaattatttatttctacttagatttttacatttgtattgttattttgatactcttatcctaaaatttttgaagttagtgttctaaattttgctgctttgtattggtaacttatatttaatattttggaacattggcttcataattttaactgtcaactgttgaatgaaaaaaaaactgtgattttggaattttggggctttttattgttgttgcgagtgtttaaatgtttaaagcaagaaatttaaggatggaaaagagtggtgattgtatttatcacaaaggtgcgtgaattttcagaggctgaaacgtactactttgaagttacttggtgatatacatcaaatacctagttatcgtctacccaaagcaatttttgcctttagtgctaactagttaaaaggtagaatgtatttttattttattttaagaaaaatgaaacctgcatgtttcttttcagagaacctgcaggtgtagacttttgtcaaatgttaatgtttgaaagcccattctcatcataagcatgataggacatcatttcacattatttgtttaaaaaaacacttctggactctagtaaatggctcctttgtttcagacattattttgattggatataatcctaatccaaaaggtataatccaaaaggtagtttgaacattctcttaatgtttgttctgtaaaccaaaaataaaattctgagccccccaactaagtggaacccgcttctctgtcaagggcattccaaagtaaacctaaaaaggtagttcaggccatgatgaaaaggtgggtggaggggattgaacatgccccgttgtaccttcctctctttggaattcaggcacaactgaccagcatcaattaaaacagagatttgaagactaacaagagtctctttgtagcaataagatatcaaatcccaacctgactctaatatagaatcactcgacagatagcaggccctgagagaaattgaaatatcttaccccaaaatatatttctttgaaatggccttgcaaagctgtctctcgtggggaagatccacattctgtacagaatcccctttcctttccagatcttttcctaattcagcagcgatttaaccaagagcctggaatattttaaggagtaataagagacatttacaaactattctctctgaagcctgctacctggaggcatcatctagataatcagaaccttggcttccacatcctcctcccttgtcttaactacaaacatttctttctgctgacttcaactcctcaggtagagtttaaccgtttcaaccaattgccattaggaaatctttaaatccacctatgcacctatgacctggacaccccccagccttccacccctgcttcaaaatgtcctgcttttctgggctgaattaatgtataccttacatctattgatttatgcctctgcctgtaatttctgtctccaaaatgtataatcaagctgtaacccaactaccttgaggacattttctcaggacctgttgagactgtgcctcaggccttgttcactcatttttggctccgaataaatcccttcaaatattttacagagtttgactgttccttgactgttctaaattaattagtgtaaatatctcagagtgttcttgaatgtacttttttccctgaagtttactgtaagatttttatttttctttagacatgggaaagtctctttctcatttgcctttgcattcaagcaaagaagatgcttatgatggagtcacatctgaaaacatgaggaatggactggttaatagtgaagtccataatgaagatggaagaaatggagatgtctctcagtttccatatgtggaatttacaggaagagatagtgtcacctgccctacttgtcagggaacaggaagaattcctaggggtatgtgttattgtattgttttccctttaaatgattttaggtatttgctcaagtattataaaaactgtgggtctcaggaaccacctgttttagaggactcttagaattaaagttgaaacatcatatcagttaccttcagtcaaaaagatgcaaaagaaacaaataaagtattaattaaggttcctaaagcagtaaagaaaaccacctcactacctccagtattttttcattcccacctgctattatagcagatattcactgtcttgtaaatgaaaacttttagtcctatttggcatctgtagagatgacatcatagttacttgctactttgctatatcagatgttagaagcaatttaagtaatttgttgacctattttttttttttttttaagagatgagatctcgcaattgttggccaggctggtcttgaactcctgtcctcaagcaatcctcccacctcggcttcccagagtgctaggattatagcagtaagccaccatgcccagccagagttaatgtgtaaggaagcttattctttcatgttatttttaaaattttatttttttaataataaggatggagtttccccatgtttcccaggatgatgtcgaattcttgggctcaagcgatcatccagccttggcctcccaaagtgttagaattacagctgtgagccaccacgcctggccaacctaatatttttaattcattatctaaagaagaagaataagctggctgtgccctaatgagttaaagaaactctgtgaagagggatggtacgtgaaaatgagccaaattgaaaaaaaaaaattgtttgtgatatatcataggggacgtcagaaggaaataatcccctgtgtgctgcttgtaagctactttaggaacagtggcaataagtagaaataataaagatactttatgctgttctcattatcttcttggtgaaagttgatgtgtgaccctggagatacccagagggaagaaggtcaaagtataaagtatactttgtgtacctttttaagatgcttaaaatgtatcttaaaatgctgtgggtttgaattgaagtttacatttgcgttggcctttgagtatgatggttttattttcacaaggatagtagctatttaagaataaaataattattgtagttgaagggattaatgcaccaggctcagggggtaggggagtactgttttagcagtctggttacatttttgaggtatgaccagtaaacttttttggccgcctggatacagattatggaaagtaggaaatcaaggatgactccagagtttttttgggggggtttgtttgagcaactgaaagaatgtagtagcaatttattaaaattgaaaaaaaaaagattatttgagtgtatatgtcacaggagagttcaggagctcaagtttagacatggatatttggagatgcctatttgacgtggaagtggatatgttcattaggcagttagttatcataagggtcaggaattcaattgcagcgcccagcctcaagttaccaatgtatagatgacacttaaagctcagctggatgacagcagcaagctagtcagtgcagatggcaaaaatgagaccctagcgaatcctgagacactgtagcatatcaaggtcagtgaaagaattaacaaaggaccctgagaaagaaaagttagaatgagaggaggaaatcattgtgtgtggtatcatggaagataagtgaagaaagtgtaccaaggaggaaagagtgaccaactgtcatatgctactgatagattgggtagtgtgaagactgagaactggccattggatttggttatatggaggccacagaagagtttaaaagagcggttttggtaaagacgtgtagcatgtatagagtgggctcaagagtgcataggagatgttacatagagagaaaatagattttctgaggggtttggctccatagcagagagaagagaggtagtcattagaaggggctgtgaggtcaggagagggatttttttttttctttgtaagatagagtgaataatagacagttgtatactgatgagaattatctgtagagaaaacattggtaattcgggagataagagaattattggagcaatgttcttgaatagacagaagggcagatctactgcacaagaacaaagagctgctctttttttctataaaaggtaagttataagcacaggtggtttattcttggtaataaaagagaaggtaaagtatggacacagaagcaggtaggtggatagatctgataatagaagcttgtagaaattatcttccatttatcacataatgaactaagaatgaggatagcgaagaagatattaaagatttgaagagagttgaagttataaaattgtggtcttggagagtcagcttgagagtatgaacttgggaaatgtagtatgattgttgacatcattaagtgctacttgacatgagtgatcaaaaatgaaaccagtgagtgtagttgtgtgtttttctgtgtccatgtttagctctgttgatatatgcataggagccagagggttggttttaaccagggttgtagttttagccagatgaataccactaaacaaggtgagatatgtaaacacacaaagatacagatatccatgttatatgtatttgtataattatatatgggtgtgatgggggagcacagatgagaggaagagtatagatgtgtcgggggaaataacagaagcattttagaaagcttttaaagagaacttgactatctgataccttaatagttaatatgataagccaacttctaataagattataaaattggcatttaaaggtccatttatcctttaaacttatggatatagctcaacccaagaaagaaatcacggattttgaagtgactttaacatggattggggtgagacctgaaagggccctggaaggctcattatgtagggggctcttaaagcttgagacttctacttcagaggccggttttggttttcaagggggacaatgtcagaagctctgcagtgatagagatgatagaggtttcaggaaagggcgataccaatatgtttcttcttttatcatccagacaaccaaatcagagtcttaatggacaccagtgaagctgaaggaaggcctggtaaatttagctcttctgaaagctaggacttagattttttttagtggaagaaaaagtaagccctcatttgttatacttttttttttttttttttagctagacatttacgtgtgaaaaaacacgctgtcttctctagaacttctgctggatactttctgaagacttagcaatttactaaaggaaagagatgagtgccagatgatattctggtctttccaaaccagattgtgatggggagccattatatttttaactaatcatagtaatttctgaacttacttctttcacatttagggcaagaaaaccaactggtggcattgattccatatagtgatcagagattaaggccaagaagaacgtaagtgattctaagaatatggcagtgttttatgttttatttttgttttttgtattttttcaaattatgtataataactagttaaaactattaaaagaaaaaatgctgtcacgtagttaaattatgtcatcatatgctttgtatccttaaacagacaagaaaggattttctggttggggtaaggtcttgaaagcagtatggtatctttatcttctcaaatctatggaaatagatctagaatagcagtacaaacacacacacacataaaattttcattaaaaattggataataaggtgtactccacaaccccaggataaaaattggtatttaaacatggtgacagcagggaccccaacggccttagcagctgtgtggaggttgtgaaggaaagcaaaaagagttactaaggatcatatgagccctgatacacaaagcaaatactttgtcccaaaagaagaggacacccagcaaatatttccaagaataatctgtaaaaatgacaagatttgcatgctctagcttggagaaagtgcaaaaagaccacaagagacctgctaagtatcttcgagatcagaaggaactggaccatcctaaatcctcagagacgttaggaaatgtggaagcaaaaattattgcaaaaggatacaaatacatatcctcaagaaaaaagttgagtagaatccaatgtttacaggacaggagttaggaaggaaggagagaacagattgacatattagggagatcatctcaaaactgtggagataaatatacatggaggccatattttcaaatactttgtatatcaacagaggatggagcccttgaacagtgaagctagcaaagcaacgctggcatattccatcaccgcaaatgaaaccttttcttgaaagtacaaaagctcttttcatttaaacatgagcacctggaaaggattgcaacaaagcattatacaaatatactttttaagaagttgaagaaagtaagctaagcaaaatactgacactttaataaaggcatgtgaaaagagcagatgaacactttaacttaatactataaaagctataacaggacagcataaggcagaaattgagaagctcagaaatgagataactaaacaatgggagtatgtgcaaagagaatttacagacctctggaaagaattagaagaaaaacatttccacaaaactggaagaacacaagtacaaatacattggaaatacagtcagggaaatagaacataaacgggaggaatgaaaacatcaacaacaaattgtgtaaaggatttgagagaaaatgtctaaaagtcacataaagaaaactaaacatgtatgtaattggagcatctgaagaagaaaaccaaaggaatagaactggacaaatgcaaaacattaaatcaagaacacagggacctgcatattgaaaagacaccacaatcctgaaaaaaatcaacccaggttagtgaaattcttgaactttaagaaaacaacaaaactcctaaatacctaggcaaaaggatcaattaatttataaatgttagaaaatcagattgacatgctgcttcttgactacagtgcctcaaacggataagcaatggtgtgacatatttaaatcaggaaaataaaacaaaccaaggattttataaacagtctgaccttttattacaaaatgtatagacagatatgaatatgaaggaaattaggattaatccctttataacctaaaagtgggagaactttttaaactatgattttgaatgcagaaaagataaatttgacagatgaacttttacaggatgaagacaaaaccttaactgaagtcaaaatacaaatagcaaactaagtagtaaagtttacaaatcatatcacagacagagggccaatatccttactctataaagagcttcagaatttgagaagaaaaaggccaacctccaaagcaaaatagaagctatgaacagctagttcacaaaaaagaaatgcaaatgacctttaaacatttgaaaagatgtccaatcttgctcataatagaaatgcaaattaaagctacacttagatgccatttctatcacagtggcaaaaatccaaaatgttgacaacatactttgttggcagtagtgactgaaaacaagtatattcattccttgctagtgaatgaaaatttggcattatttgccaaattacatctgcatttactttttggctcagaaatcctactttgaaaaatttattctcaggatagttgtcaaatatagtaaatggtatttgcgcaaattatttattgtgccaatgtttgcagaagcaaacaactggaaacaatgtaaataccaataatagaggactgatggaataagatactatgtgtacacaatggtaaaatggaaggagaactatattttaccacagtgtgatattgacaatatgtttttctttgaaaacaccacacactcaaacccacacatatacacacaaagagacaaacaatgggatgataaaccaaatctaatacaattggttcacctctgagtttgggaaggaatttggaagagggagtggataagcaagtaaagtgtgtgtgtgtgtgtgtgtgtgtctgtatctgtgtacattttttttacataacttaaaagatgaaatcaaaagtaactgccaaaaattgagacaaaaggaaacattattctaactgtatatgaagttggtgacactaacaaaactgaaataattattttaagtgacttgaaaacattgtgctgtgactataaatgtcgggagttgtgttccaaatggcaaagagaatcacaacaaattttaaactgtacttaatagttgcattattaggagtacctttgatgtagtttggaaattttatacttaaataagttattatgtaaatgccttagggagcaagagttttagcaataagaccaaagagatacaaaatcaaagaaattgaattttccacttttgacccaaatagaataatgcctgaaataccccacctgaaatcactgtaaaactggacaaatatagtaaacaatattttcagacattggacatcagccagcacagggcagtgattcctgagaaagcaaaaacaaaggagataagccctatgattgcaccagcaagccctgtgattgtaccagcttactgtctgagaaaggttccagcctgcagcacaaggaaggaaccaacccaggcagagctcagtggtcttcctcaatggagatggaactgggtgtccagaaagccaaaatgactagagttagcagggcagagtaccagaaggtagagagctggagagggtgctcttaagcctgcagctgtgtcctgatcaacatatatgtgtgaggaacatacctgaggctgaggaaagaccacccaataggagcagagagaatagtcctcagagctcacacaaggccaagaacagttgttgtttctcaatttgtaataaaattataatattttgggaacaactttgggtctttatttctttacaaattgagtgatttgtaagattattagaaaattatatttatcaattgcttttgtctgtatactttagctatgtcatttaatcctcaaacatggattttaaagaggaagaaaagtaaattgcattaaatactgcaggtaggaaatggcaaaaatggaattctaacttgattgcagatataccacacaactttcataacttcattagcaccttataactcccaggatttggttgctatcactagagccataagaaaaatcattactagttaattttgtaatggagacatatattttgtcagtaaactagccaacttttcaatttggaatgaaatactagatggaatgttgttttattgtcccaccaggttcagtaatgttcgttatggctacaaacgtggtatacagagtaatttagaacatgattcatttattcacaactaggactgacttataagccctaaggactagattatgatggattcacccgcccataagtaattaaaaataaaacgctgaagtgtagaaaatccaacaaagttcagattttattatgatgatgttaaacacttcttatgcctttttatgtattttggtgtgttatagatactataaacgtgtttaaatttcctattaggtaatccagtggggatcactacccctactcaggttgagagagtccaaaatgagtgagtgggaactgttttgattgctcaagacaacagcatggacttagtagcagtgacttagcaaatttgtgtttgcttgtattattttgttttttaataaaccagtttgctctgttcttaacttgggcaacttaaaacactaagaaatatttataaaatatctgcaggaggcactttaattgctcttgtaatacatacaaagattaaatccatcagagcatttagaaaggaaatggtggcggagagtggaagaaacaataaaatggaaaatttttgtataagtagattgtaaggaaaataagtaattgtcatgcaagactcaaaaacaaaaaaaggatgttgtgggcatccaatagaggacttgtatgttagagagaacaggaaatagttatttgaactctggttttgaggttaaccttgaaaagaggaagtttttacctacaaatttttccttgtcttatacctgcatgaaatatcttttcttgattagaaaaagaaatgtaacaacaccttcaaagcagagttgatcaatgtaaaaaaaatcttaaaaactgagtatgtaagtactttgagatgagaatactcaaaaaaagactaattatagtaagaatcattgatgagccatgtggttgtgtcatttttttttaattttaaaaatatggaggatgtgcaggtttgttacataggtaaatgtgtgccatggtggtttattgtacccatcaacccatcccctaggtattaagctcagcatgattagctatttttcctaatgctctcccttccccatcattattatttctaagatggttctgatgagcagtcactttaggggatcatgtaatggcaaaacttaacatgtgaaaattctgcttcagtgaattcagagggagttttgaaatgtgcgtttgtatgaagctcctcagcttgaatctaatgtgaaaactgctctggcagtcaaaaaagtaaatgttctattgtttttaagcccaaaatttcagtgtccaaaaatgtgattccttttttttttttttttggtacagagtctcgctctctcgtccaggcaggagtgcggtggtgcgatctcagttcactccaacctctgcctcccgggttcaaactattctcctgcctcagcctcctgagtagctgggattacaggcgcacaccaccacacctggctaatttttgtgtttttagtagagatggtttttcactgtgttggtcaggctggtctcgaactcctgacctcttgatacgcccgcctcagcctctcaaagtgctgggattacaggcgtgagccaccgcatcccgccgatttcatcttttaaagagaacatcttaacacttaatagactcagactcaaccatacccaatactttcagatgtgaaaatttggtaacatttagcatcttatatatgttgctgctgatatatgggggaaattgaacataccttctctaatgttgtgttatataatttctgatgcacatgtacttaaataccctcttttccttttcagaaagctgtatgtgatggcttctgtgtttgtctgtctactcctttctggattggctgtgtttttccttttccctcgctctatcgacgtgaaatacattggtgtaaaatcagcctatgtcagttatgatgttcagaagcgtacaatttatttaaatatcacagtgagtataaatttatatgaaaaatgtttaacttcattcttttatcctattaagcagcacctttgtccccattgagaagagatgtttgttaaatgtgacactggtcagtgtgctttctgtatgtcttttctgtattgacgttctctaccatttctggttctgctaaaaatagcaataataaatactggcaattctgtatatacagaatgatttagcatttgaaaatgtctttaggagggtaaaataaactaaaaaaagaaaaaaggtattacaatttacttttcagcatatgactctgaataacttctagcaaagtgttgacatagttgatttgatagattttataaatgtaacattttgatttcattgtatttgggaaatacagtttttgccacgtttaatagaccattcctttatcagctttaaagatgaaatacaggtgactggtttctcttttgtaaattagcatatgtgaaaatgccgtgtttttttttttttaattattaagttctagggtacatgtgcacagcatgcaggtttgttacatatgtatacaggtgccatgttggtgtgctgcacccattaactcatcatttactttaggtatttctcctaatgctatccctcccccatccctccacctcacgacaggcctcggtgtgtgatgttcccctccctgtatccaagtattctcattgttcaattcccacctatgagtgagaacacgtggtgtttggttttctgtccttgtgatagtttgcttagaatgatggtttccagcttcatccacgtccctacaaaggacatgaactcatccttttttatggctgtatagtattccatggtgtatatgtgccacgttttcttaatccagtctataattgatggacatttcggttgtttccaagtctttactattgtgaatagtgctgcaataaacataacgtgttaatgtgtctttatagcagcatgatttataatcctttgggtatatacccagtaatgggattgctgggtcaaatggtatttctagttctagatccttgaagaatcaccacactgtcttccacaatggttgaactagtttacagtccctccaacagtgtaaaagtgttcctatttctccacatcctctctaccatctgttgtttcctgaactttttaataatcgccattctaattggtgtgagatgatatctcattgtggttttgatttgcatttttctgatggccagtgatgatgagcattttttcatgtgtctgttggctgcataaatgtcttcttttgagaagtgtccgttcatatccttcgcccactttttgatggggttgtttgatttttttaagttctttgtagatcctggatattagccatttgtcagatgggtagatagcaaaaattttctcccattctttaggttgcctgttccctctgatgatagtttcttttgctatgcagaagctttttactttaattagatcacatttgtcaattttggcttttgttgccattgcttttggtgttttagtcatgaagtccttgcccatgcctacatcctgaatggtattgcctaggttttcttctagggtttttatggttttaggttttttaaatattaggtctaacatttaagtctttactccatcttgagttaatttttgtacaaggtgtaaggaagggatccagtttcagctttctacttatgacaagccagttttcccaggaccatttattaaatagggaatcctttccccattcttgtttttgtcaggtttgtcaaagatcagatggttgtagatgtgtggtattatttctgagggctgtgttctgttccattggtctgtatctctgttttggtaccagtaccatgctgttttggttactgtagccttgtagtatagtttgaagtgatgcctccagctttgttctttttgcaaacaatgtgtgtttcttaggacattgtttttgaaaggaccgtgtagccttgccactcatgaaaacaaaacaaacaaaaaccttagagaggttttctacctgttttacatgttaacgtttgtttgaaaagagcacggtttgctaactattgaattaggaaaagggataatatttattggccagccatgagatatatgtgagtgacagtgatctgccaggaagagtgatagttgttttttcttgcagaggttaatggtgtggttaaggttgaggtagagatgtctgtggtggtggttggaaagagggaggtttgtcaggacagtgagtgaaatggaatggtatgttaaagtggaatcactacttaattacagagaaaaaatggatcccgtggatccatttattcttattgctttctgtcaaaagttgccggtttaagggctttttctttaaaaacaacaatataggaaaatatacgtatatgtgaataaaacgtttgtaacacaatgctaattgtaaattgttttatgcaactggggaattgttgggtattttctcacttagccctttggaacaaggaacatattttcttttttatacactgtttaaaacattggccagactttttttggcctacctccaaagacttttttcatgtcaaataacataaatatgtactaataaataataaaaaaccctgtgatgttttcttttaagtaccagcaagtatatttaaatgtgaatatcatactctattttaagtaattatgcagaaattctcacattttggaatactgccacttaaactgagacagactgaacattaaatgaaaaataagattatttggatgaacattaactagatgacaatgagataagaggctgaattttctgtacattatctataagtatgttgaaaatggaactgctttgctttcttaaaatgattagtatatattagtagtactactttttagtaacatttgtctttctacttatgaaaatatttgtcattataagacatttgagaactataatgtataaagatttttacttctgctacctaggtcgaactattaccatatacatatatttacgaacaatgcatatacatttacaaatgtaatcattctcatgtgtaaaggatacacacattcattcacatatatacaaatgcacatttcaactgaggtcacagtacattttcccatgttaaatggtcttgtgtagtcttttacatgatcatattataattaattcaaccattttcttaatgaatgtttaattttactattacaaatattaggaaaacatctttgtgcttaaactttgtttccatttcaggttatttctttaggataaattcctgaaagtacatttgccaattaaagatcataggtagtttagggtctttgttaacaattatggtgatctcttttcatcttttaaattaaatggattctcactactctgagcaacaaatgtctaacctttattactaaatttttatttattctgtcttattttttctttgtcttaattctcataaggatttgtattctcaacctgtattacatcattgatcccttaaatattctaatgaaactgtggacagtacagaaaaagacatgtaccagcataattttgcatatacaaagacttccaagattcttctaaatttcaatccttggtcttatcagaagatccattgacctcaaattaactatctctttcctatcatgtctagaaatgaaattgacatgaacattaagagccaaatagataaaatgtttatatatttaaagattatttttactgtgtttgagaacatttttgaaatttctttacatttgtgactataccaatgtaaatgactccataaatgtaatcatataattgctgactctagcatattctttcatccttttttttactaaaactattttctctccttcaactcatcccatattgcatcttttcatatctttctacatattcctattaacatttatgtatactcacatattcagctttataaaaatcagtatttttaaaaaatattatttagtacttatttatgtgcatcttggttttcacatttgttaataccttgtaaaaatctttcctaattatctgacttaattgattctttttgatggcttcataatattccatataaggacttaatataatttattctttctttttatgaatagaattcattttattgtcaggattttttttcccattccaaataacactgtcattagcatcctaatgtttttatttctgtggaatcggtgctcagaagtaggattgcccgatcaaagtctatacataattctaattttaatgtatataactaaattactgatgaaatggctgtaacacttcacatatttgctagcagtatgaaaatatacccttttcccatatctgtgccaggaatagaatttggtgctttctataaattgtactagtttgaggaatttaaagcaatattccatttttacttaaataaacgttttcctaactataaatgaatttgagcattgtaaaatatttttggccatttagctttgtacctttttgatttgtttatgtatgtcctttgttcttttttcatttcctgttagagttttgtctttttcttttcaatttgtaagggctctttgcatgttatagattataaccgatgcttacaaatatttcttttatttatcatttagtgtctatggattttgtttatgttaccttttggtatgactgttacagatcttttatatatctgttattttttatttaacatttttaaattttttaatgactgttttctacagtaatttgagacaacagttgtaaattagaaatgtagaaggtgatcccagctattagttgttacttaagcatgtgaatgatgcaaccacatttctccccaaccaaccagacttgaatctagcccatttcttcttcatagtgagctgttgtaaatctctactagtgtacaagttgaaatagcaatttcttttaaacaaaaaagattattttaggtactgttgaacattttaaatggtcatctttgtatttaatagaataggaaacagcttactaatgtccatattctgaagctatacattttgttttttttaaatactgaattatgtttttattcttggcatattacatacatagtactcgtgcataaaaccaaataattggtttaattttctttgacattttggtattactttctttttcttaataaatgaagcatagctacagctgaagttactttaattttaaatacatatttgtatatttttagctaacttgtaaattttctgtgtcctttttttgtagaacacactaaatataacaaacaataactattactctgtcgaagttgaaaacatcactgcccaagttcaattttcaaaaacagttattggaaaggcacgcttaaacaacataaccattattggtccacttgatatgaaacaagtaagaatcaatcatgaaatactttgtaagagttaaatgaatgtcagctttgtatatcatataacttgaaggaggtgggggatttcctcaaaaacttgatttagaaatgtgtctctgctgggtacagtggctcatgcctgtgatccccgcaccttgggaggctgagctcagtggactgcttgagcccaggaattcgagaccagcctgggcaacatggtgaaacctcatctctacaaaaaaatacaaaaatgtgctgagtgtggtagtgtgcacctctcttcccacctactggggaggctgaggtgggaggatcactggagcccagtaggtcgaggctcgcgagccgtgatcacaccgccgtactccaaactgggtgacacagtgagatcctgtctcaaaaaagaaaaaaaagaaaaagaaatgtgtcttatacacatacaagatgaaatctttgagaatcaagcttgtaaaaggagaaaaatttctaattatttgaagttatgaagtatatctgaaatgtttgatgttttgatcttgttctctatcgaatttctccttttgcctttcagattgattacacagtacctaccgttatagcagaggaaatgagttatatgtagtaagttctgatttataataactttttattgtcaaataatgaagtgtataaaataaagtataaagagtatacatattttttaatttccgttatcagtcaaaaagagtacattatgtagtattctggcttctggtcctctctgttcctctttttcaacatatataatattcatgtatatatacacataaaatttgtttttttatttatttttcagaattagggttataccatgtcactttatttttcattactatgttactgtgttaaacttgttactatgttaaaataattataaattttcatcatcttttgaataagcgtagatacagacttttagggtaaataacaacctagttatcaaaatttcagaagatgagcaaaatatataagtaatttatagcctgcatttaaacaaccctgcaactgcagtggcatgaatacatacatgtttttcgagtaagaagtcatgaatatatcagtcattgtttgaagtatagaaaattctcaaccaacaaatgcttattatatttcttagcatctctgttagctttaagataggagggagaatttttaatttataatgtagtaacttgcatttgttcacattttaatttctttaacagtgatttctgtactctgatatccatcaaagtgcataacatagtactcatgatgcagtaagtacaaatcttttttgaaagagattttgcttgttaacattttggatttataacattggcttataatatacacaacatctttataaatgccacctcagttgggttttaagccttacaagagtgctatgagtaaatatcacccactttaaaaattacgaaatggttgttcagagaagtagtgaaagactacgtgactgctaagtagtgaatctggatgtcacaagagttcaggaaaatggaaaggatacagggaactgagccccacatccaattccttactcccttgacaaatagtcattaacagtaataagaatattattggtttgtaaattgggtgttatctaagccaataaaacattgttgattataggtttggtgttttgaccattagctgacatttgattaacctttttttctatgataagagaaccatggtcacttttaagcatataatgaacttttatatttttaacagaagataattgttttaaaatattacacttattatgtgtaattatgtctacagggctcactcagctatccatttttgttgtctgttggggaaatactccttaagaggattgtgtgcacaatattaagttatcattaatcaaatattctcttctgggagataatttttatgtgttaaagtagtctcactatggaaaaacttctaatataactattaaatgtctctcctcacttacattatttttagagttactgtgacaacaacatactttggccactctgaacagatatcccaggagaggtatcagtatgtcgactgtggaagaaacacaacttatcagttggggcagtctgaatatttaaatgtacttcagccacaacagtaaaaactggaagagatggatttaaagaagaaatatctattgatatttcctatactctcaatgaagaggtatttcctaataggagaccttaaattgaacaaacctaaagtttacacttctaagagtacagttaaaagtatgtggacctgcagttcttgtaactctccactctgtgttaatgatatatttgtactaggatcttttacttgaatctaaatttactggttgatttccttctccagcctatcccctacagggaaaagctgatacttcccctatagtacaataaataattatttaaaagtcatagctccagtcactactgaaaacataattttggtgataaacataatttgagaaacttaatttctgaatgtttttatagaaaattactgaaagtctattactcatggaagacttttaaagaataaccttttttcctgttttataaattcccattgttatatggtagtatttcagctacacaatattttagcttttagctagacatttatagcttttcatttgttgaaatggtaatcatctgcatgtttttgtcacttatttcaggttagtgattgcctaacacttataagccaaaataatctttgcaaaattccatacctaaaattttgaaagcccctaatgttttcacacatctttctgtattagttatagttttgtgaaatctttgtgtgatcttcaaacattatcatttaatgtacaatactgtaaataaactgtgcatggcttttatacagctttagtaaatgtcaaataaagtggtacagactcattacaacaagtttctcataaaaatacaataaataggaaaatgaaattcagaaacccatagactgggaataggttccagttacagcttggatctggcataaaataaatttgaaataaaatattttgatgctccatttttttatgttgcttttcatactaaagaatggtgtagacatgttttgcaactgttaggtacccagttatcaattttatcaatgttttagaggaggaaattatttttttggtagaaattgttcaagaaatccttaattgaatgtcattaaatgatggtggccaaaataaaacctatttagaaatttaatcactttgcacatcacttggaatatgatgcctctagtagttacttttttatagttttctacttttggttttatttaaaattgttttcaaatatagattattgacttattcaactttgctgttttatattttcagtatcatttttcatttttttttttttttgtcttttcacttaccaagttctagggacatttaaaatatgtactaagtgtaggagtggttatgataccaaaaaatgtagctgggttgagattaatttcgttctgttttctcatgacagaaatcaggtttccctttccccacccctaagtgcctaacttaggtctgaaacagcctgtttattagtctgactctctcaaccataaaacataagctttatttaattctgcctttaaacacactcaggtttccccttaattttcatattattttctgcaagttttcttgagtatcttcaattcgttgaatgtggtttttggtttttttttgttttaacactagtcttcccttaattcattgctaactcaagccatccttactattaaacccaaatcagtcctttaagttcattatggcctttctagtatttaaaaaaaaaaaaatcattttcatttttcttctgctacgtttcctgactactactgcatacttctctgatacaggttctgtttgtattttttatatcattctcattttctcatttgacatgatctatgtctatatatgatataggtccccttttgtctcaaaatttttaattatgtgacttcaaaaatcacctgtatctgtagtagggcttccaaatctgcttctccatatgtgaccagtcacctgtctgctttcacatttagctagtgaactacacatttactaaaatgtgtaaattttacacatttagtgactgtgtaaaataaaaaaaaagttattttatcatatcctttctattatgttcccatcctgtcctcatgtcccatttactttattatcaccattcatttcttcaaaattatcttttagatacgctcatacaaaaatcaatccttgttttcttgcttgtgtcttttaaccttggaaaattacatcgtgtaaattaaacagatttttctgatgatctgtgcttcttatatactattagagtgcatgatagtatctcctgaaaaggatggaaagtagaagcatttgcttttagtcacttaattttgaatcttttttcttcatcttttgaattaattttttttattatatctacttttagtggagtttgagtcagaaaaaaacaagaatttgaaacaagtaaaaagatagaagagaaataaagatggtatgtgactactttcagagagagttaagtaactgtcagaataagcctggaacaaaacaggctgtaaattaataaaactacaaacacacattcaggtgaagcagaagtatagccataaaacatctagaaagagtgaatgaggcttttagcttttcttaggtcaatgtccagtgtgcttttttccatgggaataggataggtattaatacgcttttctaaactgctctcagaccttatccagaggacatggtaaagatatgttacagaaatttttctgatacttcctggaataactttaagttacaccctagtagactggtcattctaataaaatccagtactataacaaacctctgtatgttgatagcacattggccctttttagagttctttcctatgtttttcttacgtgatttcccacagttccatgagtccaacaaaggagagtgataggctccttatcttttagaagaggaaggaaaggcatgaagaagttgagggactggctgaagatcacgtacttactaagtagtacaactggagcaagatcaagtatctctgtctcccatatctgtgttctatcatttaaaatatatattggaaatccctgctgactcagattggtatgattaaaaatgagaggaaagttcaaatagttagtagtgacaaactaatactgctggactaagattttggtagcattgttttctaaaatattttaaatggagaatgaacacttataaaatgctttggaacataatctttagcttaattttctgttaaaatttagtaccccttcatcattccaataaagataagactgatccattgtctaaggaaattatttataaataatagagattaatttatttgagatttgaaataagaatagtatgaaaatattagataccacataaattgtttgaaattactgaataaccatcttaagtatggaacatttaaatggctatattttatttgtgtacagtttttctgtgccttgttaggccagtgaagcaattattttctctaagaaaatgacaataaaatataacacacttcagattgtctgatttacagtttggaaaggacaccgcaatgttcaaataggtaggagaccatcaaaaacacaattaaagtaacatattaggagacttgaaacttcagcctaataaatccttcatggttcttagccttattattgtgatataattctagatattttcttggagggcatgtgcccaactctcccgcaccccattttgtttgtcttttaaagttcttagaataaacagttctttatataataattatattttatttaagaaaatagtttgttaggtactttttaaaagatgtaaatttttaaatttacaaatacatatgggtctttgataagcaataggaattgaattacaagttactagggttataagcaaaaggttgcttaccataatgtcattaggtcacgatttttagctcacatctggaagcagcaactacttggctcaagtacatataagagtaattagttttattctctcttttttataaaatcgggtttcagatgagatgtttatcttagactattttagggaaaaattttacatgtttgagatggtggagtaaaaagactgttaaacatttcttttaaaaaattatttttacattacaacaatatatttatgatgtgttcagatcaaaaatttaacttctgtgtcccagatctactttcaaagtgagattttcacttgtcagcttaaatttctgactagaactaacatttgtgtatttttgtgcttagtcggaatacaaatttcacagtggatttttgaagtttgtccttaaattggataaaatcaagtgattaaagttactaaagagataaaaatggtaatttccatttttaaaagtaatttggttgtgtttatagttatttgtacaagtatttatcacagactctaaattgaaaaatgtagtatgatctatatttgaccctaaaaatgttggattaatttaacaaatatggcagatttttcataactaagtcttaagtcttctaaaaggaagctgttacccttctgtttttaattacattaattgaaatgtgttttaagagatacaatttcagcatattttatatattaaaaaacaaaaaaggattagtattgagccagtggccaaaaggtaatattactaccatgtagactgttatagttcaaattgtcccacttcacccagaattttagaaactagaagtctgggaggtactatatcagctgtagttgggtaattccaagtgctgatagtactattcatcttttttattattgtgtcagatgaaacaaatgccaagttgcaaaatatgcagatttttattatataatggttttaggcataaattattaacaagccatgccttatgtgtttcatcttatatttttctttagaactaaactataacagattttggaaaatgatttgacgtgcttgctcacttgattgacttggtcagatatttgaatgatggtattacctagattctaatccttgattctagttatataataaataatatagaatatgaaaatatgtttgggcatttactgtttatattatgtagtagcctccatcatgacacacttactacatttatgaattgagcagttctgtaattgtaattattattgctgttcatgtaacaaaacatgcttataatagcaaacaaatagaaatgcccccaaaatgctatttttttaattcagttataactgttactcttgtagttgtgtatgacgcaataaaatttgtaaaaaaatttcagcatgaaaaataaaatttgtatcacttatgtattttggctatgcttttgaaatttccttaacataaacaaatcagtgtagatattcaaaatcttttacctttaatccttttcatccttatagtccttacctgaactctactttagcattatctttgatttctttttttcattcacttaacttccaatcagtactcaagctttatccattctacctctgtaatgtctctggaatttatctcatacgtgaaatttcccttgatattatagacataccttggatatattgcaggtttggttccacaccatatcgataaagtgatataatcaagcaagttacacacctttttttggtttcccagtgcatatgaaagttatgtttacatcatactgtagtctattaagtgtgcaatagcattatgtcgaaaaacacaatatatatgccttaataaaaaataggctgtgtgcagtggctcacacctgtaatcccagcactttcggaggccaaggtgggaggatcacctgagcccagaagttcaagaccaaattggtcaacatggcgagaccccatataaaatatatacatacacacacacacacacacacacacacacacacacacacactattgctaaaaaatgttaacgatcatctgaatgttcagaagtttatactccttttgccggcacaggatcttgtctggatgttgtttgctgatagatccatgtagtggttactaaagttggggtggctatggcaatttcttaagacaacaatgaagtttatcacatctattgccttgtcctttcatgaaagatgtctctagcatgtgatgctgttttgatagcattttaccaatggtaggtcttttttcaaaattggagccagtcctctcaaaccctgcctctgctttatcgaccatgtttaaataattttctaaattctttattgtcatttcagcagtggtctcagcatcttcaccaggagtagattccaactcaagaaaccactttcttagctcattcataagaaacaattcctcatctgttcaagttttggcatcaagttgtagcaattcagtcacatctacagactccattgctaaattttagttctcttgctatttccacatctgctgtgacttccttcactgaagtcatgaacccctcaaaattatccatgagagttgaaatcaatttctttcaaactcctgttaatgttcatatttagacctcttcccctgaatcacaaatgttgttaatggcatctagaatggcaaatcctttctggaaggtttcaatttactttgcccagatccatcagaggaatcactacctatgacagctatggtcttacaaaatgtatttcttatatagtaagattcaaaagtcaaatttactccctgatccatggctgcagaatgcacgttgtgttaagaggcatgaaaacaacattaatctatttgtacatcatcagagctgttgggtgaccaggtacattgtcaatgagtagtaatactttgaaaggaatttttttttccaagcaggtctcaacaatgggcttaaaatattcagtgaaccgtgttgtaaacagatgtcatattatccagactgttccatttattgaacacaggcagagtaggtaaaagttttaagggtcctaggattttcaggatggcaaatgagcattggcttcaacttaaagtcaccagctacattcaccactaaagagagtcagcctgtcctttaaagctttaaaggcaagcattgacttctctttagctatgaaagttttagatggcacctttttgtaaaagaaggttgttttatctacattgaaaatatattgttcattgtaaccactttcatcaatgatcttagctagatattctgggtaacttactgcaggttctccatcagcacttactgcttcaccttgcacttttatattatggaaacaacttatctccttaaacatcattaaccaacctcttggtagcttccaacttttcttcaatagcttcctcatctctctcagccttcatagaattgaagcgtgttagggctttgttcaggattaggctttcacttaaggcaatgttgtggctggtttgatctattgaaaccactaaaacattcccatatcggcaataaggctgtttgattttcttatcattcatgttttctggaacacttttcaattacttacagtaaggaaatagcacttttaatttccttcaagaacttttcctttgccttcacaacttgtgcaagagacctagctttcagcctatcttgactttccaagattgtccttggtgagaaatgcttgattactaggtgtaattatttctagcttttgattgaaagtgagagatatgtgactcttccttttacttgaacagttagaggctattgtaggattattaattggcctaatttcaatgttattttgtctcaaggagagagggatgtgggaacagctggttaggagagcagtccgaacacacacttttttttattactttcactgccttacatgggtatggttcatgctgcctccaaatacttacaaaaacatcaaagatcactgattacagatcattaaaataggtttaataataatggaaacatttgaaatattgcaaaaattaccaaaatgcgacagagacatgaaatgagaacatgctgttgaaaaaaatggcactcatagacttgttggacacaggattgaaagaaaccaattcatcaaaaccacaatatctgcaaaacgcaataaggtgatgcacaataaaaggaagtatatctgcacttccttcttttttactgaaaaagcccaagttcttactctggtgttgagaacctttcattagctggctacaatctcagttcacaatcatttcttattgcccttcaacaatgtcctgaccaaatcgagttctttctcattgggaacagttcattcctttatcccttcgctcattctgttccttttttcttagaaaaattatttgcccatgttttttatcccccaattctatctttttcaatgcccacttcgtgtccaacccctctctactttccctctaaacttactcaaacacttggtgaactttttctgaatgttttttctgaatgtttacttttaaaggagttgcccagtcacaaagtttgagccaagtttttttgttttaaacttgttttaaacttttgttttcatcaacttttttcaaacaatttttgtcagcttagaggttgtgttttaattactatttttaaaatcttgtatgaggtagtatgatttgaaattagatggaatgtgcccaaaattatctctgtagaataatatggaaaaacgaaaatgaaaagatgtattacctgaatgtgctcctcctagccagtttctcaagggagaagaatcattaataagtttgtgaggctttttttttttttctggtagatttctgtttccttgatcagtggtttttcagaattttttaaagcttgtgaaatacttatttgtaaatagcatcttatgaggaaccttgatatgtgacaaaacagatgctttgatttgagagcagaagacctgcagcccctgactgctcacctttccagggacccctgaacattggcctagagggtagtgtacagtcacttcttcagtgataacttactgagtgtgattcaacaaaactgctttggtttatgggtgaaacataatttataactaggcaaatgtcagcttaaatttatattttctaaatagcactgaaaatgacaatagacaaatgtaatttctgatttttaagaaatgattttccattttaacatacattttaaatgttttgtcttcaaagggaatccttaaaaatgatatattggaaaccactgatttcttattttcatttcatgaatttcagattcatgaatggaatatattgataatagcattttttaagttgccccaaaaagatgaaatacaaataattttaatcccaaatctatttcagacacctaactttttttttattttttatactttaagttctaggatacatgtgcagaacgtgcaggtttgttacataggtatacacgtgccatgttggtttgctgcacccatcaacccgtcatctacattagatatttctactaatgctatccctcccctagccccctaccccacaaaaggccccagtgtgtgatgttcccctccctgtgtccatctgttctcattgttcaactcccacttatgagtggcaacatgtgctgtttggttttctgttcctgtgtgtgtttgctgagaatgatggtttccagcttcatccatatccctgaaaaggacatgaactcatccttttttatggctgcatagtattccatggtgtatatatgccacattttctttatccagtctatcattgatggacatttgggttggttccaagtctttactattgtgaacagtgctgcaataaacatatgtgtgtatgtgtctttacagtaaaatgatttataatcttttgggtatacacccagtaatgggattgctgggtcaaatggtatttctggttctagatccttgaggaatcaccacactgtcttccacaatggttgcaatttacactcccaccaacagtgtgtaaaagcttcctatttctctacattctctccagcatctgttgtttcctgacttaatgattgccattctaactggcgtgagatggtttctcactgtggttttgatttgcatttctgtaatgacccgtgatgatgagctttttttcatgtttgttggctgcataaatgtcttcttttgagaagtgtaaggtacttaattcttaaattaaaaaaaaatacccggccgggcacggtggctcacgcctgtaatcccagcactttgggaggccgaggcgggtggatcatgaggtcaagagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggggcttgcagcgagccgagattgcgccactgcagtccgcagtccggcctgggcgacagagcgagactccgtctcaaaaaaaaaaaaaaaaaaaaaaaaaaaatacccttctttaacaaaatcaagtttcaagttttctgccttgttgataacttgttaacatttttttattttagaataaatctaaacagagaccacactgatatctgtaacaacctatgtaaagttaagattaaggttaaatcacatttttcacagagagtgttttactgaaatataaacttttatgcatctgatattatctaattgtgtccttactgggtaacttgtgtatttttcaagttaaaggatttcctaaaaatgcttatttgaatatttatgtcatttgtcacacatactttcaaaacataaccttcttcaacgagtatcttcttttccttcctaaaatactgttaatagctggctattttgtaagcctgtatctggtttaaaaaaaagaagtgaatattttcaatacttatgaaccatagaattctgtattcttttcataacttttaacttttccataggacctatcactatgtaaaatattatacatatctactgatttatttattgcctatgtacctatactagaacatagtaaacatgagaacagggaatcttggttattaattcctgcaggactcaactacttatcactcaaacatttgttcttgaaaagaacttcgtataacatcattagatttttacagaatctttaaaaaagctttaatattttcaaggttttttatttatataaatattgttactttaccactagtgatttcaatagtggcattgtcacatgattcaagaagaggaggccattacattactgttgtattaaaaagtcagttcaggcttaatatatatgactactataagtcaacctcacattttaatgtatgaagaagattgtatgaacgatcacattaattcatttaaatagtatgtattttgatactaatttttaagaagaaatgtatcattctcatggaaataaaagataatttgaaaagca

SEQ ID NO: 2 - DNA, 6514 bp, Homo sapiens
  source 1..6514: mol_type = unassigned DNA
aggcgcggacgcaggttacagcagcgcttggcctctgctgatgccgtcgttatcctacccctcccccgtcccagctctacggcggccgcgcgctccaggccggtcgctccaccccccggctcccgggactgtggactccacgaccctgtcctcggccctgtccgcgccgaagcagcccgggactgcgcagcgccccgcgtgccgatcttttcctaattcagcagcgatttaaccaagagcctggaatattttaaggagtaataagagacatttacaaactattctctctgaagcctgctacctggaggcatcatctagataatcagaaccttggcttccacatcctcctcccttgtcttaactacaaacatttctttctgctgacttcaactcctcagacatgggaaagtctctttctcatttgcctttgcattcaagcaaagaagatgcttatgatggagtcacatctgaaaacatgaggaatggactggttaatagtgaagtccataatgaagatggaagaaatggagatgtctctcagtttccatatgtggaatttacaggaagagatagtgtcacctgccctacttgtcagggaacaggaagaattcctagggggcaagaaaaccaactggtggcattgattccatatagtgatcagagattaaggccaagaagaacaaagctgtatgtgatggcttctgtgtttgtctgtctactcctttctggattggctgtgtttttccttttccctcgctctatcgacgtgaaatacattggtgtaaaatcagcctatgtcagttatgatgttcagaagcgtacaatttatttaaatatcacaaacacactaaatataacaaacaataactattactctgtcgaagttgaaaacatcactgcccaagttcaattttcaaaaacagttattggaaaggcacgcttaaacaacataaccattattggtccacttgatatgaaacaaattgattacacagtacctaccgttatagcagaggaaatgagttatatgtatgatttctgtactctgatatccatcaaagtgcataacatagtactcatgatgcaagttactgtgacaacaacatactttggccactctgaacagatatcccaggagaggtatcagtatgtcgactgtggaagaaacacaacttatcagttggggcagtctgaatatttaaatgtacttcagccacaacagtaaaaactggaagagatggatttaaagaagaaatatctattgatatttcctatactctcaatgaagaggtatttcctaataggagaccttaaattgaacaaacctaaagtttacacttctaagagtacagttaaaagtatgtggacctgcagttcttgtaactctccactctgtgttaatgatatatttgtactaggatcttttacttgaatctaaatttactggttgatttccttctccagcctatcccctacagggaaaagctgatacttcccctatagtacaataaataattatttaaaagtcatagctccagtcactactgaaaacataattttggtgataaacataatttgagaaacttaatttctgaatgtttttatagaaaattactgaaagtctattactcatggaagacttttaaagaataaccttttttcctgttttataaattcccattgttatatggtagtatttcagctacacaatattttagcttttagctagacatttatagcttttcatttgttgaaatggtaatcatctgcatgtttttgtcacttatttcaggttagtgattgcctaacacttataagccaaaataatctttgcaaaattccatacctaaaattttgaaagcccctaatgttttcacacatctttctgtattagttatagttttgtgaaatctttgtgtgatcttcaaacattatcatttaatgtacaatactgtaaataaactgtgcatggcttttatacagctttagtaaatgtcaaataaagtggtacagactcattacaacaagtttctcataaaaatacaataaataggaaaatgaaattcagaaacccatagactgggaataggttccagttacagcttggatctggcataaaataaatttgaaataaaatattttgatgctccatttttttatgttgcttttcatactaaagaatggtgtagacatgttttgcaactgttaggtacccagttatcaattttatcaatgttttagaggaggaaattatttttttggtagaaattgttcaagaaatccttaattgaatgtcattaaatgatggtggccaaaataaaacctatttagaaatttaatcactttgcacatcacttggaatatgatgcctctagtagttacttttttatagttttctacttttggttttatttaaaattgttttcaaatatagattattgacttattcaactttgctgttttatattttcagtatcatttttcatttttttttttttttgtcttttcacttaccaagttctagggacatttaaaatatgtactaagtgtaggagtggttatgataccaaaaaatgtagctgggttgagattaatttcgttctgttttctcatgacagaaatcaggtttccctttccccacccctaagtgcctaacttaggtctgaaacagcctgtttattagtctgactctctcaaccataaaacataagctttatttaattctgcctttaaacacactcaggtttccccttaattttcatattattttctgcaagttttcttgagtatcttcaattcgttgaatgtggtttttggtttttttttgttttaacactagtcttcccttaattcattgctaactcaagccatccttactattaaacccaaatcagtcctttaagttcattatggcctttctagtatttaaaaaaaaaaaaatcattttcatttttcttctgctacgtttcctgactactactgcatacttctctgatacaggttctgtttgtattttttatatcattctcattttctcatttgacatgatctatgtctatatatgatataggtccccttttgtctcaaaatttttaattatgtgacttcaaaaatcacctgtatctgtagtagggcttccaaatctgcttctccatatgtgaccagtcacctgtctgctttcacatttagctagtgaactacacatttactaaaatgtgtaaattttacacatttagtgactgtgtaaaataaaaaaaaagttattttatcatatcctttctattatgttcccatcctgtcctcatgtcccatttactttattatcaccattcatttcttcaaaattatcttttagatacgctcatacaaaaatcaatccttgttttcttgcttgtgtcttttaaccttggaaaattacatcgtgtaaattaaacagatttttctgatgatctgtgcttcttatatactattagagtgcatgatagtatctcctgaaaaggatggaaagtagaagcatttgcttttagtcacttaattttgaatcttttttcttcatcttttgaattaattttttttattatatctacttttagtggagtttgagtcagaaaaaaacaagaatttgaaacaagtaaaaagatagaagagaaataaagatggtatgtgactactttcagagagagttaagtaactgtcagaataagcctggaacaaaacaggctgtaaattaataaaactacaaacacacattcaggtgaagcagaagtatagccataaaacatctagaaagagtgaatgaggcttttagcttttcttaggtcaatgtccagtgtgcttttttccatgggaataggataggtattaatacgcttttctaaactgctctcagaccttatccagaggacatggtaaagatatgttacagaaatttttctgatacttcctggaataactttaagttacaccctagtagactggtcattctaataaaatccagtactataacaaacctctgtatgttgatagcacattggccctttttagagttctttcctatgtttttcttacgtgatttcccacagttccatgagtccaacaaaggagagtgataggctccttatcttttagaagaggaaggaaaggcatgaagaagttgagggactggctgaagatcacgtacttactaagtagtacaactggagcaagatcaagtatctctgtctcccatatctgtgttctatcatttaaaatatatattggaaatccctgctgactcagattggtatgattaaaaatgagaggaaagttcaaatagttagtagtgacaaactaatactgctggactaagattttggtagcattgttttctaaaatattttaaatggagaatgaacacttataaaatgctttggaacataatctttagcttaattttctgttaaaatttagtaccccttcatcattccaataaagataagactgatccattgtctaaggaaattatttataaataatagagattaatttatttgagatttgaaataagaatagtatgaaaatattagataccacataaattgtttgaaattactgaataaccatcttaagtatggaacatttaaatggctatattttatttgtgtacagtttttctgtgccttgttaggccagtgaagcaattattttctctaagaaaatgacaataaaatataacacacttcagattgtctgatttacagtttggaaaggacaccgcaatgttcaaataggtaggagaccatcaaaaacacaattaaagtaacatattaggagacttgaaacttcagcctaataaatccttcatggttcttagccttattattgtgatataattctagatattttcttggagggcatgtgcccaactctcccgcaccccattttgtttgtcttttaaagttcttagaataaacagttctttatataataattatattttatttaagaaaatagtttgttaggtactttttaaaagatgtaaatttttaaatttacaaatacatatgggtctttgataagcaataggaattgaattacaagttactagggttataagcaaaaggttgcttaccataatgtcattaggtcacgatttttagctcacatctggaagcagcaactacttggctcaagtacatataagagtaattagttttattctctcttttttataaaatcgggtttcagatgagatgtttatcttagactattttagggaaaaattttacatgtttgagatggtggagtaaaaagactgttaaacatttcttttaaaaaattatttttacattacaacaatatatttatgatgtgttcagatcaaaaatttaacttctgtgtcccagatctactttcaaagtgagattttcacttgtcagcttaaatttctgactagaactaacatttgtgtatttttgtgcttagtcggaatacaaatttcacagtggatttttgaagtttgtccttaaattggataaaatcaagtgattaaagttactaaagagataaaaatggtaatttccatttttaaaagtaatttggttgtgtttatagttatttgtacaagtatttatcacagactctaaattgaaaaatgtagtatgatctatatttgaccctaaaaatgttggattaatttaacaaatatggcagatttttcataactaagtcttaagtcttctaaaaggaagctgttacccttctgtttttaattacattaattgaaatgtgttttaagagatacaatttcagcatattttatatattaaaaaacaaaaaaggattagtattgagccagtggccaaaaggtaatattactaccatgtagactgttatagttcaaattgtcccacttcacccagaattttagaaactagaagtctgggaggtactatatcagctgtagttgggtaattccaagtgctgatagtactattcatcttttttattattgtgtcagatgaaacaaatgccaagttgcaaaatatgcagatttttattatataatggttttaggcataaattattaacaagccatgccttatgtgtttcatcttatatttttctttagaactaaactataacagattttggaaaatgatttgacgtgcttgctcacttgattgacttggtcagatatttgaatgatggtattacctagattctaatccttgattctagttatataataaataatatagaatatgaaaatatgtttgggcatttactgtttatattatgtagtagcctccatcatgacacacttactacatttatgaattgagcagttctgtaattgtaattattattgctgttcatgtaacaaaacatgcttataatagcaaacaaatagaaatgcccccaaaatgctatttttttaattcagttataactgttactcttgtagttgtgtatgacgcaataaaatttgtaaaaaaatttcagcatgaaaaataaaatttgtatcacttatgta

SEQ ID NO: 3 - DNA, 32695 bp, Macaca fascicularis
  source 1..32695: mol_type = unassigned DNA
ctccgggagcactgtgcgcaggcgcgggagcaggttacggcggcgcttggcctctgctgatgccgtcgttgttctacccctcccccatcccagctctgcggcggccgcgcgctccaggccggtcgctccaccccccggctcccgggactgtggactccgtgaccctgtcctcggccctgtccgcgccgaagcagcccgggactgcgcagcgccccgcgtgccggtgagcgaccccggacgtgcagtccccaggtccccttccggagcggggctgagcggctgcgctgcggcttcagccgggcctgacgaggaggggaggccgcctgggtgttgggcctgccgcaggtacggcggcgctcagggctcgagatcgaggtgctgtttcgcctcttttgaggcccagccagggaacaccggccttcggcccaagcccggcctctgggttgcctctaggccctcaccccttgagcgccaggtggccctcttgattttgctgttgatgaattttcttgccgtggtgccgggatgttatttttcagctttacagagtagactttgtgctaggtgttgtttatggagctttttaaaggcgctggtttccttcgttctcaagcccttttagaactaaaaccagtgtctgcacagacgatcaggagccatttaaattggagtgctgtgatatattttttctctccaaattgagtctgatatccatgttggctttcagtaaaggaaggaaccgctataataacattgagcaccgagaggttggagaagagggtctagcaccccttttttaaaccattgagtctccaagaataaaagtggttaaacttgtcagttctctaaggaaaatctagtagagaaggcaagggattcccagattttgacaaagtctggcacttgtccaaacacctacctaaaacaatcgatcacggcagctgttagattaggacagaaaacaggcccaaacggagagccacttgagttttcctttctgtgtcttttctctcaacgccctccagtaagatgcagcttacaccctggctgtgttgactgagatcccagaaagggtgcctctaaggctgaaagggtgcccttaagaccttgctggtaaggcccagtgatcccagttcatctagtgttccgcatagaccccaaggtcactgtgtggggacagtgatgttaagtcttctctaacctacctgtggctggcaggtgttgatttgaccagggtattgtgtcattgagccaaattaagacaatgattttttagaagtcataaatcatttgtatttttaatgtgggcaattatttttataaatacatagttttttttttttttaaagagtgaaaatcgcattttacagtttcgtttgcacaccatttgtatattttttttattctgtttgacagcaacaagtggatggcatgcgagttttttttttttttatctttgtatttattttgtatatatattcatgggcaatttgctacaagtgcaatttgctacattgatatacgacctcctagtgaagtcagggccctcagtgcatccaacactggagcaacacacattgtacccaccaagcaacctcccatcaggcatgccaattctaaaaagtaaaagtttcttcatactctaggatgaattgacttatttggagtatctgttttgtttaatcaaataagtgagaatttttgaaagaaatactatctgtcttaatgtctgtaatctatttttaggaaccgataaaagacatatcaaatttagaaggagtagaattatgttacttctaagttttactttttcactttattttttctcaagttataagcaggaaaatgccagcagggtgtattgtatgactattaagttcttttcagctttaaaagggtatgaccttaagtataaagttacttcattatctacttgcagaagttatttttgtgtaggcactagtcctaattatttctacttagatttttacatgtgtatttaattttcccatgttatttggatactctcatcctaaaagtttgaagttagtattctaaattttgctgctttgtaaattatatttaatattttggaacactggcttcataattttaactctgtcaactgctgaatgaaaaaaacttgcatgattttggaatttcggggctttttattgttgttgcaagtgtttaaatgtttaaagcaagaaatttaaggatggaagagtggtgattgtatttatcacaaaggtgcatgaattttcagaggctgaaacgtactgttttgaagttacttggacatatacatcaaatacctagctatcatctacccaaagcaatttttgcctttagtgccgactggttaaaaggtagaatgtatttttattttattctaaggaaaatgaaacctgcatgtttcttttcagagaacctgcagatgtagacttttgtcaaatgttaatgtttgaaagcccattctcatcataagcatggtaggatatcatttcacattattttctttaaaaaacacttctggactctagtaaatgactcctttgtttcagacattattttgattggatattatcctaatccaaaaggtagtttgaacattctcttaaagtttgttctgtaaaccaaaaataaaattctgagccccccaactgactgagtggaccctgcttctctgtcaagggcattccaaagtaaacctaataatgtggttttggccatgatgaaaaggtgggcggaggggattgaacataccttattataccttcctctctttagaattcaggcacaactgaccagcattgatgaaaacagagattttaaaactaacaaaagagtctctttgtagcagtaagatatcaaatcccaacctgactctaatatagaatcacttgacagatagcaggccctgaaagaaatcaaaatatcttaccccaaaatatatttatttgaaatggccttgcaaagctgtctcttgtggggaagatccacattctgtacagaatcccctttcctttccagatcttttccttaatccagcagagatttaaccaagagcctggaatattttaaggtgtaataagagacatttacaaactgttctctctcaagcctgctacctggaggcatcatctacataataagaaccttggcttccacaacctcctcccttgtcttaactataagcatttctttctgctgacttcaactcctcaggtagagtttaacccctttcaaccaattgccattagaaaatctttaaatccacctttgcacctatgacctggataccccccaaccttccacccctgcttcaaactgttctgcttttctgggctgaattaatgtataccttacatgtattgatttatgcctctgcctgtaatttctgtctccaaaatgtataaatcaagctgtaacccaactaccttgaggacattttctcaggacctgttgagactgtgcctcaggccttggtcactcatatttggctcagaataagtcccttcagatattttacagagtttaactcttcctttactgttctaaattaattagtgtaaatatctcatagagttcttgaatgtacttttttccttgaagtttactgtaagatttttatttttctttagacatgggaaagtctctttctcatttgcctttgcattcaagcaaagaagatgcttatgatggagtcacatctgaaaatatgagaaatggattggttaatagtgaagtccataatgaagatggaagaaatggagatgtctctcagtttccatatgtggaatttacaggaagagatagtgtcacctgccctacttgtcagggaacaggaagaattcctaggggtatgtgttattgcattgctttccctttaaataatgatttcaggaatttgctcaaatattataaaaactatgggtctcaggaaccacctgttttagaagactgttagaattaaagttgaaacatcatatcagttatcttcaggcaaaaagatgcaaaagaaacaaataaaaaaatattaattaaggttcctaaagcagtgaagaaaaccacctcactacctccaatattttttcattcccttctactattataacagatattcactgtcttgtgaacaaaaacttttagtcctatttggcatctatagagatgacatcatagttatttgctactttgctatatctgatgttagaagcaatttaagtaatttgttgacctaatttttttttttttaagagatgagatctcgcaattgttggccaggctggtcttgaactcctgtcctcaagcaatcctcccatcttggcctcccagagtgctaggattatagcagttgagccccatgcccagccagaattaatgtgtaaggaagcttattctttcatgttattttaaaaattttattatttttaataataaggatagaatttccccatgtttcccaggatgatgatgagttcttgggctcaagccatcatccagccttggcctctcaaagtgttagaattacagctgtgagccaccacgcctggccaacctaatatttttaattcattatctaaagaagaagaataagctgcctgtgctgtaatcagttatttctaaaagaaactctatgaagagggatggtatgtgaaaacaagccaaattgaaaaaaaaaaaaattgtttgtgatatatcacaggggacgtcagaaggaaataatcccctatgtgctgcttgtaagttactttaggaacagtggcaataagtagaaataataaagataccttatgctgttctcattatcttcttggtgaaagttgatatgtgaccctggagacacccagagggaaaaaggacaaaatgaaagtataaagtataccagagtcttaaaaatgctgtgggttggaattgaagtttacatttgcattggcctttgagtatgatggttttatcttcacagggataatagctgtttaagaataaaataattattgtagttgaagggattaatgcaccagactcaggggttaggggagtactgttttaccagtctggttacatttttgaggtgtgactagtaaacttttttggccacctggatacagattatggaaagtaggaaatcaaggatgactgcagagttttttgtttttttaggtcgtttgtttgagcaactgaaagaatgtagtaacaatttattaaaattgaaaaaaatattatttgagtgtatatgtcacaggagagttcaggagctcaagtttacacatgtaaatttggagatacctatttgacatggaaatgggtatgttcattaggcagttagttatcataagggtcaggaattcaattgcagggcccagcctcaagttatcaatgtatagatgacacttaaagctcagctggatgacagcaccaagctaatcagtgcagatagcaaaatgagaccctagcactgaatcctgagacactgtggcatatcaaggtcagtgaaagaattaacaaaggaccctgagaaagaacagcgagagaggaggaatcactgtgtctggtatcatggaagataagtgaagaaagcgtaccaaggagaaaagagttaccaaatgttggatgctactgatagattaggtagtgtgaagactgagaactgaccattggatttggtgacatggaggccacaggagagtttaaaagagcagttttggtaaaggcatgtgtagcatgtatagggtgggctcaagagtgcatagaagatgttacatagagagaaaacagattttctgaggagtttggctccatagcagagagaagagaggtggtccttagaaggcgctgtgaggtcaggagaggatttttttaaatatatgtatatatataagatggagtgaataatagacacttgtatgctgatgagaattatctgtaaacattggtaattcaggagataagaggaattattggagtaatgttcttgaacagatgaaagggcagatctgctgcacaggagcaaagagctgctcttttttctataaaaggtaagttgtaaacacgggtagtttattcttggtaataaaagagaaggtaaagtatggacacagaagcagctacgtggatagatctgatagtaaatcttgtagaaattatcttctgtttatcatataatgaactaagaatgaggatagcgaagaagatattaaagatttgaagagagttgaagttataaaattgtggtcttggagagtcagcttgagagtatgaacttgggaaatgtagtatgattgttgacttcattgagtgctacttgacatgagtgatcaaaaatgaaaccagttgtgtatttttctgtgtccatgtttagctgtgttgatatacgcacaggagccagagggctggttttaaccagggttgtagttttagccaaatgaataccgctaaacaaggtgagatatgtaaacacacaaaaacgcagatatccatattgtatgtatttgtataattttgtataattatatatgggtgtgatgggggagcacagacgagaggaagagtatagaagtgttggagggaataacagaagcattttagaaagcttttaaagataacttgactatctggtaccttaatagttaatataataggccaacttctaataagcttataaaattggcatttaaaggtccgtttatcctttaaaaacttatggatttagctcaacccaagaaagaaataatggattttgaagtgactttaatatgaattggggtgagacctgaaagggccctggaagactcattatgtagggggctcttaaagcttgagacttctacttcaaaagccagttttggtttttgagggggacaatgtcagaagctctgcagtgatagaggtttcaggaaagggagatagcaatatgtttcttcttttatcatccagacaaccaaatcagagtcttaatggacaacagtgaagcagaaggaagacctggtaaatttagctcttctgaaagctgagactttttaaattttttttcagtagagaaaaagtaatggagccctcatttgttacaatttttttttttaagctcgacatctaggtatgaaaaaacacactgtcttctctagaacttctgctgtgtactttctgaagacttagcaatttactaaaggaaagagatgagtgccagatgatattctggtctttccaaaccagattgtgatggggagccattgtatttttaactaatcatagtaatttctaaacctacttctttcacgtttagggcaagaaaaccagctggtggcattgattccatatagtgatcagagattaaggccaagaagaacgtaagtgattctaagaacatggcagtgttttatgttttatttttgttttttgtattttttcaaattatgtataaacaactctagttaaaactattaaaaggaaaaatgctgtcacgttgttaaattatgtcatcatatgctttgtatccttaaacagacaaacaaaaaaggaatttctgtttagggtaaggtcttgaaagcagtatggtatctttatcttctcaaatctatggaaatagatctagaatagcagtaggaaacacatacactcacataaaattttcattaaaaattggataaggtatgctccacaaccccggaataaaaattggtatttaaacatgatgacaacagggaccccagctggcttagcagctgtgtggaggttgtgaaggaaagcaaaaagagagttactgaggatcctatgagccctgatacacaaagcaaatactttgtcccaaaagaaaaggacacccagcaaatatttccaagaataatctgcaaaaatgacaaggtttgcatgctgtagcttggagaaagtacaaaaataccacagagatgtactaagcatcttcgagaagatcagaaggaactggaccatcctaaatcctcagcgaccttaggaaatgtcaaaacaaaaattcttggaaaaggatacagatacatatcctcaagaaaaaagttgttgagtagaaaacagtgtctacaggacaggagttaggaatgaaggagagaacagattcagatattagggagatcatctcaaaactgtggagataaatacacatggaggccatgttttcaaatactttgtatgtcaacagaggatgtatcccttgaacagtgaagctagcaaatggacgctggcatattccatcaccaaaaatggaaccttttcttaaaagtacaaaagctcttttcatttaaacatgagcaactggaaaggatcgcaacaaagcattatacaaatatactttttaagaaattgaagaaagtaagctgagcaaaatactgacactttaataaaggcatgtgagaagagcagatgaacactttaacttaatactatatatagaagctacaacaggacagcataaggcagaaattgagaagctcagaaatgagataactaaacaatcagagtatgtgaaaagagaatttacagacctctggaaagaattagaagaaaaacacatccacaaaactggaagaacacaagtacaaatacgttggaagtacagtcagggaaatagaacataaacaggaggaatgaagacatacacaacaaattaatgtaaacgatttgagaacaaatgtctaaaagtcaaataaactgaacatgtgtgtaattggagcatctgaagaagaaaaccaaaggaatagaactgaacaaatgcaaaacattaaatcaagaacacagagacctacatattgaaaagacatcacaatcctgaaaaaaatcaacccatgttagtgaaattcttgaactttaaggttaagaaaacaacagcaaaactcctaaatacccaggcaaaaggatcagttaatttataaaggttagaaaatcagattgacatgctgctgcttgactacagtgcctcaaacagataagcagtggagtgatgtatttaatcaggaaaataaaacgaaccaaggattttataaatagtctgaccttttattacaaaacgtataaacagatatgaatatgaaggaaattaggatgaatcccttcaaaacctacaagtgggagaactttttaaactatgattttaaatgcagaaaaggtaaatttgataagtaaacttttacagaatgaagacaaaaccttaactgaagtcaaaatacaaatagcaaactgagtagtaatgtttacaaatcatatcacagacagagggccaatatccttactctaaaaagagcttcagaatttgagaagaaaaaggccaacctccaaagcaaagtagaagctatgaacagctagttcacaaaaaagaaatgcaaatgacctttaaacatttgaaaagatgtccaatcttgctcataatagaaatgcaaattaaaactacacttagttgccatttctgtcacattggcaaaaatccaaaatgttgacaacatactttgttggcagtagtgactgaaaacaagtatattcattccttgctagtgaatgaaaatgtggcgttatttgccaaattacatctgcatttactttttggctcaggaatcctactttgaaaaatttattctaaggatagttgtcaaatatagtaaattgtatttgcacaaattattcattgtgccaatgtttgcagaagcaaacaactggaaacaatgtaaatacccataatagaggactgatggaatgagatattacatgtacacaatgataaaatggaagaagtattttactacagtgcgatattgagaatatgtttttctttgaaaacaccacacactcacacccacacacatatacacacaaacagactgacttataagccctaaggactaaatagattatgatggattcacccgcccataagtaattaaaaataaaacactaaagtgtagaaaatcggataaaaccaaatcaaataaaacaatgggatgataaaccaaatctaaagaattggttcccctcagagtttgggaaggaatttgaaagagggagtggataagcaagagtgtgtgtgtgtgtgtgtctgtgtacatatttttttacattaaagatgaaatcaaaagtgaaagtaactgccaaatattgagataaaaggaaacattattctaactgtatatgaaattggtgacactaacaaaactgaaagattttaagtgacttgaaaacattgtgctatgattgtagatgtttagtaagttgtgttccaaatggcaaagaaaatcacaataaattttaaactgtacttaatagttgtattattaggagtacctttggtgtaatttggaaattttatatgtaaataagtaattatgtaaatcccttaggaagcaagatttttagcgttaagagcaaagagatacaaaatcaaagaaattgaattttccacttttgacccaaatagaataatgcctgaaatacttccacctgaaatcactaaaaaaatggacaaatatagtaaacaatattttccaacattggacatcagccagcacagggcagtgattcctgagaaagcaaaaacaaaggagatacgccctataattgcaccagcaagccctgtgattgtaccagcttactgtctgaggaaggttccaagctgcagcacaaggaaggaaccaacccaggcagagctcagaggtcgtcttcaattgaggaggtggatctgagtgtccagaaaaccaagctgactagagtttgcagggccgagtaccagatggtagagagctgcagagggtgctcttaagactgcagctgtgtcctggtcaacatatatgtgtggggaacatacttgaggctgaggaaagactacccaataggagcagagggaatagtcctcagagctcacacaaggccaagaacagttgtttctcaatttgtaataaaattataatattttaggaacaactctgggtccttattcctttacaaattaagtgagttgtggtgagattagaaaattatcagttgcttttgtctgtatactttagctatgtcatttaatcctcaaacatggattttaaagtggaagagaagtaaactgcgttaaatactgtgggtaggaaatggcaaaaatggaattctaacttgattgcacatataccacacaactttcataactttatcagcaccttataactcccaggatttgggtgctatcgctagagccgtaagaaaaatcattgctaattaattttgtaatggagacatatattttgtcagtaaactagccaatttttcaatttggaatgaaatactacatgaaatgttgttgtattgtcctactcaggttcaataatgtttgttatggctacaaccttgatatacagagtaatttagaacatgattcatttattcacaactaggactgactcataagccctaaggactaaatagattatgatggattcacccacctataagtaattaaaaataaaaccctaacgtgtagaaaatccaacaaagttcagattttattatgatgttaaattcttcttatgcttttttatgtattttggtgtattgtagatactataaacgtgtttaaatttcctattaggtaattcagtggggatcactaccctactcaggttgagagagtccagaatgagtaagtgggaactactttgtttgctcaagacaacagtatgggcttcatagcagtgatttagcaaatttgtgtttgcttatattattttgttttttaataaaccagtgtgctctgttcttaacttgggcggcttaaaacactaagaaatatttaaaaaatatctgcaggaggcactttaatagttgttgtaatacatacaaagattaaatccatcagagcatttagaaaggaattggtggcgaagagagagtggaagaaacaataaaatggaaaatttttgtataagtagaatgtaaggaaaataagaaataattgtcatgcaagactcaaataaaaaaggatgttgtaggcatccagtaaagggcttgtatattagagaggacaggaaatagtcctttggaactctgggtttgaggttaaccttgaaaagaggaaatttttacctacacatattggaaagagcaagaaaaggaagaaatacatttcaggagagttagaagaatgtcaaaggcatgtaaggcttagtttcttccttgtcttatacctgtgtaatatatcttttgattaaaaaaagaaatgtaacaacaccttcaaagcagagttgatcaatgtaaaaaatattttaaaaactgagtacataagtactttgagatgagaatactcaaaaaagagactatagtaagaatcatttattaggcatgtggttatgtcatttttttttttttaatatggaggatgtgcaggtttgttagataggtaaatgtgtgccatggtggtttgttgtacctgtcaacccatcccctaggtttttaagcccagcatgattagctatttgtcctcatgctctcccttccccatcattatttttaagatggttctgatgagcagtcactttaggggatcatgcaatggcaaaactcatgtaaaaattccgcttcagtgaattcagagggagttttgaaatgtgtatttgtatgaagcgcctcaacttgattctaatgtgaaaactgctctggcagtcactaaagtaaatgttctattgcttttaagcgcaaaatttcagtgttcaaaaatgtgattccatttttttttgagatggagtctcgctgtcttgtctaggcaggagtgcggtggcacgatctcggctcactccaacctctagctcccgggttcaagctattctcctgcctcagcctcctgagtacctgggatatcaggcacacatcaccacgcctggctaatttttgtgtttttagtagagacggggcttcactatgttggtcagactggtctcgaactcctgacctcgtgatccacccacctcagcctctcaaagtgctgggattacaggcatgagccacagcgtgccgccgatttcatcttttgaagggaacatcttaacacttagtactcagactcaatgatacccaatactttcaaatgtgaaaatttggtaacatttaatgtatgttgctgctaatatacgggggaaattgaacatatcctctctagtgttacattatataattcttgatgcacatgtacttaaataccctcttttccttttcagaaagctgtatgtgatggcttctgtgtttgtctgtctgctcctttccggattggctgtgtttttccttttccctcgctctatcgacgtgaaatacattggtgtaaaatcagcatatgtcagttatgatgttcagaagcgtacaatttatttaaatatcacagtgagtataaatttatatgaaaaatgtttaacttcattcttttatcctattaagcagcacctttgtccccattgagaagaggtgtttgctaaatgtgacactggtcagtgtgctttctgtatgcctttgctgtattgaccttcagtaccatttctggttctgctaaaaatagcaataataaatactggcaattctgtatatgcagaatcatttagcatttgaaaatgtctttaggagggtaaaataaactaaaaagagaaaaggtattacaatttacttttcaacatttgactctgaataacttctagcaaagtgttgacatagttgatttcatagattttataaatgtaacattttcatttcattgtatttgggaaatgcagtttttgccacctttaacagaccattcctttatcagctttaaagattacatacaagtgactggtttctcttttgtaaattagcataggtgaaaatgccctgtggttttttttaattatactttaagttctagggtacatgtgcacaacgtgcaggtttgttatatatgtatacaggtgccatgatggtgttctgcacccattaacttgtcatttacattagatatttctcctaatgctatccccctaccccaccacagaccccggtgtgtgatgttccccaccctgtgtccagatgttctcactgttcagtttgttcagaatgatggtttccaacttcatccatgtccctgcaaaggacatgaactcatccttttttatggctgcatggtattccatggtatatgtgtgccacattttcttaatccagtctatcattgatggacatttcggttggttccaagtctttgctattgtgaatagagccgcaataaacatacatgttcatgtgtctttatagcagcatgatttataatcctttgggtatatacccagtaatgggatggctggatcaaatgatatttctagttctagatccttgaggaatcgccacactgtcttccatagtggttgaactagtttacactcccaccaacagtgtagaagtgttcctatttctccacatcctctccagcatctgttgtttcccgaactttttaatgactgccattctaactggtgtgagatgatatctcattgtggttttgatttgaatttctctgatggccagtgacgatgagcattttttcatgtgtctcttggctgcataaatgtcttcttttgagaagtgtctgttcctatcctttgcccactttttgatggggttttttaatttttttcttgtagatttatttaagttctttgtagattctggatattagccatttgtcagatatgtaggttacaaacattttctcccattctgtaggttgcctgttcactctgatggtagtttattttgctgtagagcagctctttaattagatcccatttttcaattttggcttttgttgccattgcttttggtgttttagtcatgaagaccttgcccatgcctatgtcctgaatggtattgtctaggttttcttctaggatttttatggttttaggtctaacagttaagtctttaatccatcttgaattaatttttgtataaggtataaggaagggatccagtttcagctttctacatatggctagccagttttcccagcaccatttattaaatagggaatcctttccccatttattgtttttgtcaggtttgtcaaagatcagatggttgtagatgtgtggtattatttctgagggctctgttctgttccattggtgtatatctctgttttgataccagtaccatgctgttttgattactgtagccttgtagtatagtttgaagtgatgcctccagctttgttttttttgcaaacaatgtgtgtttcttagaacattgtttttggaaggactgtgtagccttgccactcatgaaaataaaacaaacaaaaagctcagagaggttttctacctgttttacatgttaacatttgtttgaaaagagcatggtttgctaactgttgaattaggaaaagggatgatgtatattggccagccgtgaaatatatctgagtgacagtgatctgccaggaagagtgatagttgttttttcttgcagaggtgagtggtatgattaaggttgaggtagagatgtctatagtggtggttggaaagagggaggtttgtcgggacagtgagtgagacggaatgttatgttaaagtggaatcactacttaattgcagagaaaaagtaactcccgtggatccatttatccttattgctttctgtcaaaagttgccagtttaagggctttttctttaaaaacaatataggaaaatatatatatatatatatatgtgaataaaacatttataacacaatgctaattgtaaattgttttacacaactggggaattgttgggtatattctcagccctttggaacaaggaacatattttcttttttgtacactgtttaaaacattgaccagactttttttggcctacctccaaagccttttttagtgtcaaagaacataaatatgtactactaataaaaagccatgtgatgttttcttttaagtaccagaaagtatatttaaatgtgaatatcatactctgttttaattatgcagaaattctcacattttggaatactgccacttaaactgagacagattgaacattaaatgaaaaataagattattatttggatgaacattaagtttaactagacgacaataagatgctgaattttctgtacattatcagtattttgaaaatggaactgctttgctttcttaaaattagtacatattagtagtactactctttagtaacatttgtctttctacttacaaaaatatttgtcattataagacatttgaaaactataatgtataaagatttttacttctatctaggtcgaactattaccacatacatatatttatgagcaatgcatatacatttacaaatgtaatcattatcatgtgtaaaggatacacacacactcattcacatatatacaaatgcacatttcaactaggtcacagtacattttcccgtgtcaaatggtcttgtgtagtcgtttccgtgatcatattgtaattaattcaaccattttcttaatggatgtttaattttgctattacaaatattcagaaaacacctatatgcttaaactttgtttccatttcaggttatttctttaggataaattcctgaaagtatatttgccaattaaagatgataggtagtttagggtctttgttaataattacagtgacctcttttcatcttttaagttaaatgaattctcactactctgagcaacaaatgtctaacctttattactaaatttttatttattgtcttattttttctttgtcttaattctcataagcatttgtattctcaacctgttttatgtcattgatcctttaaatattctaatgaaacttatggacagtacagaaaaagacaggtaccagcataattttgcatatacaaagacttccaagatttttctaaatttcaatccttggtcttatcaggagatccattgacctcaaattaactatatctttcctatcatgtctagaaatgaaattgacatgaacattaagaaccagctaaaacgtttatatatttagataaaggttatttttactatgtttgagaactgttttgaaatttctttacatttgtgactataccaacgtaaatgactccataaatgtaatcatataattgctgactctagcatattcgttcatccttttttactaaaactattttctctccttcaactcatcccatattgcatcttttcatatctttctccatattcctgttaacatgtatatatatactcacatattcagctttataaaaatcagtattttaaaaaaatattattaagtacttacttatgtacatcttggttttcacatttgttaataccttgtaaaaatctttcctgattatctggcttggttctttttgatggctccataattttccatataaggacttaatataatttattcattgttttatgaatagaattcattttattttcaggattttttttcccatttcaaataacactgtcattagcatcctaatgttttcatttctgtggaatagttgctcagaagtaggattgctagatcaaagtctgtacatagttctaattttaatgtatatgactgaattactgatgaaatggctgtaatacttcacatatttgctagcagtataaaaatatacccttttcccatatctgccaggaatagaatttgttgctttctataaattatactagttcaaggaatttaaagcaatattccatttttacttaaattacctttttcttaactgtaaatgaagttgagcattgtgaaatatttttggccatttagctttgtacctttttgatttgtttatgtatattctttgttcttttttcattttctgttacagttttgtcttttcaatttgtaagggccctttccatattctagattataactgatgcttacaaatatttcttctatttatcatttagtgtctactgattttgtttatgataccttttggtatgactgttttagattttttatatatctgttattttttatttaacatttttaaattttttaatgactgttttctacagtaatttaagacgataattgtaaattagaaatgtctaaggtgatcccagctattagttcttacttaagcatgtgaatgatgtaaccacatttctccccaaccaaccagacttgaatctagcccatttcttcttcataatgagctggtgtaaatctctactaatgtataagttgaaatagcagtttcttttaaacaaaaaagatgattttaggtactattgaacattttaaatgaccatctttatatttaatagaatagcaaacagcttactaataaagtttgtggagacatgtatatatattgagttagtaaaaatgtagattaaatcttaaaattatttttattaaacatttgctaaggccacaaataaattttctgacattaatttgtccatattctgaaactatacattttctgtttttttaaatgttgaatcatgtgtttattcttggcatattacatacatagtactcatgcataaaaccaaataattggtttaattttctttgacattttggtattactttctttttcttaattaatgaagcatagccacagctgaagttactttctttaattttaaatacatatttgtatgtttatttttagctaacttgtacattttctgtctccttttttgtagaacacactaaatataacgaacaataactattattctgtcgaagttgaaaacatcactgcccaagttcaatttgcgaaaacagttattggaaaggcacgcttaaacaacataacccatattggtccacttgatatgaaacaagtaagactcaatcatgaaataccttgtaagagtgaaatgaatatcaactttgtatatcatataacttgaaggaggtgggggatttcctcaaaaacttgatttaaaaatgtgtctccgctgggtatagtggctcatgcctgtgatactagcacccttgggaagctgagcttggtggactgcttgagcccaggaattcaagaccagcctgggcaacatggtgaaacctcatctctacaaaaaatacaaaaatgtgctgagtgtggtggtgtgcacctctcgtcccacctactggggaggctgatgtgggaggatcacttgaacccagtaggtcgaggcttgcaagccgtgattgcactgctgtactccaaactgggtgacacagtgagatcctgtctcaaagaaaaaaaaaagaaaaagaaaagtgtcttgtagacttacaagatgaaatctttgagaatcaagcttgtaaagggagaaaattttctaattctttgaagttatgaagtatatctgaaatatttgatgttttgatcctgttctctattgatttttttcccttttgccttacagattgattacacagtacctaccgttatagcagaggaaatgagttatatgtagtaagttctgatttataactttttattatcaagtaatgaaatgtataaaataaggtattaagaatatacatattttttaatttccataatcagtcaaaaagagtacattatgcagtattctggtttctggtcctctctgttctttttcaacatatataatattcatgtatatatacacataaaatttgtttttttatttttcagaattagggtcatatataccatgtcactttatttttagttactatgttaactatgttactatgttaaacttagttactatattaaaataattataaattttcatcatcttttgaaagcttagatatagaattttagggtaaataacaacccagttatcaaaatttcagaagatgagcaaaatatgtaagtaatttatagcctgcatttaaacaaccctgcagctgcagtggtgtgaatacatacatgtttttctagtaagaaggcatgaatatatcagtcattgtttgaagtatagaaaatgctcaaccaataaatgcttattatatattcttaggtaacatctgttagctttaatataggaaggagaatttataatttataatgtaataacttgtatttgttcacgttttaatttcttttaacagtgatttctgtactctgatatccatcaaagtgcataacatagtactcatgatgcagtaagtacgaatcttttttgaaagagatatttcttgtcaaaattctagatttataacattggcttataatatacacaacatctttataaatgccacctcagttgggttttaagccttacaagagtgctgtgaggttgagtaaatatcacccactttaaaaatcaggaaatggttggtcacagaagtagtgaaatactacattactgctaaatagtgaatctggatgtcacaagagttcaggaaaatggaaaggatacggtgaactgagccccacatccaattccttactcccttgataaatagtcattaacagtaataagaagtagtaatattattggtttgtaaattgggtgttatctaagccaataaaacattgttggttacaggtttggtgttttgaccattagctgacatttgattaacctttttttctatgataagagaaccatggtcacttttaagcatataatgaacttttatatttttaacaagataattgtcttaaaatattacatttattatgtgtaattatatcaacagggttcactcagctatccatttttgttgtctgttggggaaatgttccttaagaggactgtgtgcacaatattaagttatcattaatcaaatattctcttctaggagataatttttatatgtaaaagtagtctcattatggaaaaacttctaatataactattaaatgtctctccccactaacattatttttagagttactgtgacaacaacatactttggccactctgaacagatatcccaggagagatatcagtatgtcgactgtggaagaaacacaacttatcagttagggcagtctgaatatttaaatgtacttcagccacaacagtaaaaactggaagagatggatttaaagaagaaatatctattgatatttcctacactctcagtgaagaggtatttcctaataggagatcttaaattgaaccaacctaaagtttacacttctattttaagagtacagttaaaagtatgtggacctgcagttcttgtaactctccactctgtgttaatgatgtatttgtactaggatcttttacttgaatctaaatttactgtttgacttccttctccaacctatcccctacagggaaaagctgatacttcccctatagtacaataaataattatttaaaagtcatcgctccagtcactactgaaaacataattttggtgataaacataatttgagaaacttagtttctgaatgtttttatagaaaattactgaaagtctattattcatggaagacttttaaataaccttttttcctgttttataaattcccgttgttatatggtagtatttcagctacacaatattttagctttcagctaaacatttatagcttttcatttgttgacgctgtaatcatctgcatgtttttgttacttgcttcaagttagtgattgcctaacacttgtaagccaaaataatctttgcaaaattccatacctaaaattttgaaagctcctgatgttttcacatgtctttctgtattatagttttgtgaaatctttgtgtgatcttcaaacattatcatttaatgtacaatactgtaaataaactgtgcatggcttttgtacagctttagtaaatgtcaaataaagtggtacagattcattacaacaagtttctcgtaaaaatacaataaataggaaaacgaaattcagaaacccatagattgggaataggttccaattacatgttggatctggcataaaataaatttgaactattttgatgctccattttttttatgttgcttttcatgctagagaatggtgtatacatattttccaactgttaggtacccagttatcaattttatcaatgttttacagaaggaaacttttttttgatagatactgttcaagaaatcctgtcattaaatgatggtggctatatcaaaataaaacctatttagaaatttaatcactttgcacatcacttggaatatgatgcctctagtagttacttttttatagttttctacttttagttttaattttatttaaaattgtattcaaatatagattattgacttactttgctgttttatattttcaatatcatttttcatttaattttttttttcacttaccaagttctagggacatttaaaatatgtatcactaagtgtaggagtgatgataccaaaaaatgtaggtgggttaagattaatttcattctgttttctcgtgacagaaatcaggtttccctttccccatcccctaagtgcctaacttaggtctgaaacagcctgtttgtttattagtctgactctctcaagcataaaacgtaagctttatttgtttaattctgcctttaaacacactcaggtttccccttaattttcatattattttctccaagttttcctgagtgaatatcttaaattcgttgaatgtgctttttctttctttttacactagtcttcccttaattcattgctaactcaggccatccttactattaaacccaaatcagtcctttaagttcattatggcctttcctagtataaataaataaataaataaatacaaaaaaaaccttcattttcatttttcttctgctatattttctgactactactgcatacttctttaatacattgattctgtttgtttttttttttttttttttttgagaaggagtctggctctgtcacccgtgctggagtacagtggctggatctcagctcactgcaagctccacctcccgggttcacgccattctcctggctcagcctcctgagtagctgggactacaggtgcccaccacctcgcccagctagttttttgtatttttttttagtagagacgaggtttcactgtgttagccaggatggtctcgatctcctgacctcgtgatccgcccgtctcggcctcccaaagtgctgggattacaggcttgagccatcgcgcccggcctctgtttgtattttttatatcattctcattttctcatttgacatgatctatgtctatatatgatataggtccccttttgtctcaacatttttaattatgtgacttcaaaaatcacctgtatctctagtagggcttccaaatctgcttctccatatgtgaccagtcacctgtctgctttcacatttagctagtgagctacacatttactaaaatgtgtaaattttacacatttagtgactgtataaaatcaaaaaaatgttaaagacattttatcatatcctttctattatgttcccaccctgtcctcatgtcccgtttacttcattatcaccattcatttaaaattatcttttagatatgctcatacaaaaatcaatccttgttttcttgcttgtgtcttttaaccttagaaaatttcttgcttatgtcttttaaccttggaaaattacattgtgtaaattaaacagattttcctgatgatctgtgcttcttatatcctattagagtgtatgcatagtatctcctgaaaaggatggaaagtagaaaaaacatttgcttttaagtcacttaattttgaatcttttttcattttttgaattaaattttttttttttttactacatctagtttcactggagtttgagtcagaaaaaaacaagaatttgcaacaagtaaaaaagatagaagagaaattaagatggcatgtgactactttcagagagaattaagtaactgtcagaataagcctggaacaaaacaggctgtcaattaataaaactacaaacacacattcaggtgaagcagaagtatagccataaaacatctagaaagagtgaatgaggcttttagcttttcttaggtcagtgtccaatgtgctttttcccatgggaataggataggtattaatacacttttctaaactgctctcagaccttatccagaggacatggtaaagatatgttacagaaatttttctgatacttcctgtaacttaaatcttttaagttacaccctagtagactggtcattctaataaaatccggtactgtaacaaacgtctgtatgttgatagcacattggccctttttagagttctttcctatgtttttcttacatgatttcccacagttccgtgagtccaacaaaggagagtgataggctccttatattttagaaggggaaggaaaggcatggagaagttgagggactggctgaagatcccatacttaactaagtagtataattggagccagaccaagtctctctgtctcccatatctgtgttctgtcgtttaaaatatatattggaaatccctgctgactcagattggtatgattaaaaatgagaggaaagttcaaatagtagtgacaatcaaactagtactgctggactaagattttggtagcatctaaaatgttttaagtggagaatgaacacttataaaatgctttagaacataatctttagcttaattttctgttaaaatttagtaccccttcatcattccaataaagactgatccattgtctaaggaaattatttataaataagagattaatgtatttgtgatttgaaataagaatagtatgaaaatattagataccacataaattgtttgaaattactaaataaccatcttaagtatgggaacatttaaatggctatattttgtgtacagttttcctgtgccttttttgttaggccaatgaagcaattattttctctaagaaaatgacaataaaatatagcacacttcagattgtctgatttacagtttggaaaggacactgcaatgttcaaataggtaggagaccatcaaaaacacaattaaaataatgtgttaggagacttgaaactttagtccaataaatccttcatggttcttagccttattattgtgatataattctagatatttttttggagggcatgtgcccaactctcccccaccccattttgtttgtcttttaaagttcttagaataaacagttatttatataataattatattttatttaagaaaataatttgttagatactttttaaaagatgtaaatttttaaatttacaaatacatatgggtctttgataagcaatatgaattgaatttcaggttactagggttataagcaaaaggttgcttaccataatgtcattaggtcatgatttttagctcacatctggaagcagcaactacttggctcaagtacatataaaagtaattagttttattctctcttttttataaaattgggtttcagataagatgtttatcttacactattttcttttagggaaaaattttacatgtttgagatggtggagtaaaaagactgttaaacatttcttttaaaaaattatttttacattataacaatatatttatgatgtgtttagatcaaaaatttaacttctgtgtcccagatctactttcaaagtgaaattttcatttgtcagctgaaatttctgactagaactaacatttgtgtatttttgtgcttagtcagaatacaaatttcacagtgaatttttgaagtttgtccttaaattggaaaaatcaagtgattaaagttactaaagagataaaaatggtaatttccatttttaaaactaatttggttgtgtttatagttactcgtacaagtatttatcacagactctaaattgaaaaatatagtatgatctatatttgaccctaaaattgttccataaattaacaaagatatggcaaatttttcataactaaatctaagtcttctaaaaggaagctgttacccttctgtttttaattacattaattgaaatgtgtttaagagatgatttcagcatattttgtatattaaaaaaaaaaaaggattagtattgggccaatggccaaaaggtaatattactaccatgtagattgttatagttcaaattgtcccacttcccccagaattttagaaactagaattctgggagatactgtatcagctgtagttgggtaattccaagtgctgatagtactattcatcttttttattactgtgtcagatgaaacaaataccaagttgcaaaagatgcagatttttattatataatggttttaggcataaattattaacaagccatgccttgtatgtttcatcttacatttttctttagaactaaactataatagattttggaaaacgatttgatgtgcttgctcacttgattcacttggtcagatatttgaatgatggtattacctagattctaatccttgattctagttatataataaataataatatagaatgtgaaaatatgtttgggcacttactgtttatattatgtagtagcctccatcatgatacactacatttatgaattgagcagttgtgtaattgtaattattattgctgttcatgtaacaaaacatgcttatgatagtagcaaacaaatagaaatgcccccaaaatgctatttttttaattcagttgtaactattactcttgtagttgtgtatgacacaataaaatttgtaaaaaaaatttagcatgaaaaataaaatttgtatcactgatgtattttggctatgcttttgaaatttccttgacataaacaaatcagtgtagatattgaaaatcttttacctttaatctttttcatccttatagtccttacctgaactccactttagcattctctttgatttctttcattcatttaacttccagtcagtgctcaagctttatcccttctacctctgtaatgtctctggaatttatctcatacatgaaatatcccttgatattatggacataccttggagatactacaggtttggttccaccccatatcaataaagtgatgtaataaagcaagttacacacctatttttggttttccagtacatacaaaagttaggtttatatcatactatagtctattaagtgtacaatagcatgtcaaaaaacacaatatacatgccttaataaaaaaatagtctctgtgcagtggctcccacctgtaatcccagcactttcagaggccaaggtgggaggatcacctgagcccagaagttcaagaccaaactggtcaacatggcgagaccctatataaaatacacatgcacacacacacacccacacacacacattattgctaaaaaatgttaacaatcatctgaatgtacagaagtttgtattacttttgctggcagaggatcttgtctggatgtcgtttgctgacatccatgcagtggttactaaagttggagtggctatggcaatttcttaagacagcaatgaagtttatcacatctattgtcctttcatgaaagatttctctagcatgtgatgctgttttgatagcattttaccaatgatatgtcttttttcaaaattggagccagtcctctcaaaccctgccactgctttatcaaccatgtcaaccatgtttaaataattttctaagttctttattgtcatttcagcaatggtctcagcatcttcaccaggagtagattccaactcaagaaaccactttctttgctcattcataagaaaattcctcgtctgttcaagttttgtcatcaagttgtagcaaacagattccattgctaaattttagttcccttgctatttccacatctgctgtgacttccttcactgaagtcgtgaattttgagggcaaaattatccatgagagttgaagtcaacttctttcaaactcctgttaatattcatatttagacctcctcccctgaatcacaaatgttcttaatggcatctagaatggcaaatcttttctggaaggtttcaatttactttgtccagatccatcagaggaatcactacctatgaagctatggtcttacaaaatgtatttcttatatagtaagattcaaaagtcaaatttactccctgatccatggctgcagaatgcatgttgtgttaagaggcatgaaaacaacattaatctacttgtacatcatcagagttgttgggtgagtagtaatattttgaaaggaattcttttttccaagcagtaggtctcaacaatgggcttaaaatattcagcgaaccgtgttgtaaacagatgtgctactatccagactgttccatttattgaacacaggcagagtaggtaaaagttttaagggtcctaggattttcaggatggcaaatgagcattggcttcaacttaaagtcaccagctacattcaccactaaagagactcagtctgtcctttaaagctttaaaggcaagcactaacttttctttagctatgaaagttttagatggcacctttttgtaaaagaaggttgttttatctaaattgaaaatatgttgtttagtgtaaccactttcatcagtgatcttggctagatattctggataacttactgcaggttctccatcagcacttactgcttcaccttgcacttttatattatggaaacaacttatttccttaaacatcattaaccaacctcttggtagcttccaacttttcttcgatagcttcctcgtctctctcagccttcatagaattgaagcgtgttagggcttcggtcaggattaggctttcacttaaggcaacgttgtggctggtttgatctatccagatcactaaaacattcccatatcaacaataaggctgtttcattttcttattattcatgttttctggaacacttttcaattacttctagtaaggaaatagcacttttaatttcattcaagaacttttcctttgcattcacaacttgtgcaagagacctagctttcagcctatcgtggctttccaagatagtcctttgtgagaaatgcttgatcactaggtataattatttctagcttttaattgaaagtgagagatatgtgactcttccttttacctgaagatttagaggccattgtaggattattaattggcctaatttcaatgttattttgtctcaaggagagagggatatgggaacagctggttattagagcagtccggacacacactttttttttactacgttcactgccttacatgggtgtggttcatgctgtctccaaatacttaaaaaacataaagatcacggattacagatcattaaaataggtttaataataatggaaccttttgaaatattgcaaaaattaccaaaatgcagagagacatgaagtgagaacatgctgttgaaaaaatggcactgatagacttgctggacacaggattgaaagaaaccaatttatcaaaaccacaatatctgcaaagcacaataaggtgatgcacaataaaaagaggtatgtctgcatttccttcttttttactgaaaaaagccaaagttcttactctggtgttaacctttcattagctggctacaatctcagttcacaatcatttcttattttccttcaactatgtcctgaccaaatagagttctttctcattgggaatagttcattcctttatccctttgctcattccattcctttttccttagaaaattatttgcccatgttttttatcccccaattcacatgtccaacccctctctacttgtctccctccaaacttactcaaacacttggtgaatttttttctgaatgttttttctgaacatttacttttaaaggagttgcccagtcacaaagtttgaactcaagtttttttttgttttgaactcaaaacttgttttaaacttttgttttcatcaacttctttatttaaacaatttttgtcagcttagagtttgtgttttaattactatctttaaaatagtaattatcagatagtatgatttgaaattatatggaatgtgcccaaaattatctctgaagaataatatggaaaaatgaaaattaaaagatgtattacctgaatgtgctcctcctagccagtttctcaagggagaagaatcattaacaagtttgtgaggctttttttttctggtagatttctgtttctttgatcaatggttttttcagaatttttttaaggttgtgaaatacatatttgtaaatagcatcttgtgtagaatcttgatatatgacaaaacagctgctttgatttgagagcagaagacctgcagacccagactgctcacctttccagggacccctgaatcattggcctagagggtagtgtacagtcacttcttcaatgataacttactgagtgtgattcaacaaaactgctttgtttatgggtaaaacaatttataactaggcaaatgtaggcttaaatttatattttataaatagcactgaaaatgataatagacaaatgtaatttctgtaatttctgatttttaagaaatgattttctgttttaacatatattttaaatgttttgtcttcaaagggaatccttaaaacatatactggaaaccattgatttcttattttcatttcatgaatttcagattcatgaatggaatatattaatagcattttttaagttaccccaaaaagatgaaatacaaataattttaatcccaaatctatttcagatacttaactcttattttttatactttaagttctgggatacatgtgcagaacatggtttgtcacataggtatacacatgccatgttggtttactgcacccatcaacccatcatctacattaggtatttatcctaatgctatccctcccctaggatcccaccccacgacaggccccagtgtgtgatgttcccctccctgtgtccatgtgttctcattgttcagctcccacttatgagaacgtgtgatgtttggttttccgttcctgtgttagtttgctgagaataatggtttctagcttcatcaatgtccctgcaaaggacatgaactcacctttttttatggctgcatcgtattccatggtgtatatgtgctacattttctttatctagtctatcattgatgggcatttgggttggttctgagtctttactattgtgaacagtgctgcagtaaacatacatgtgcatgtgtctttatagaagaatgatttataatcctttgggcatatacccagtaatgggattgctgggtcagatggtatttctggttctagatctttgaggaatcaccacactgtcttccacaatggttgaactaatttacactcccaccaacagtgtgtaatttctctacattctctacagcatctgttgtttcctgagttaatgattgccattctaactggtgtgagatggtatatcattgtggttttgatttgcatttctctaatgatcagtgatgatgagctttttttcatgtttgttggctgcataaatgtcttcttttgagaagtgtaagatacttaattctttaatttaaaaaaataccctccccaaactaacaaaatcaagttttctgccttgttaataacttgttaacattttttaattttagaataaatctaaacagagaccccattcatatccgtaacaacctatgtaaagtaagattaaggttaaatcacatttttcacagagagtgttttactgacatacaaacttttatgatctgatattatctaattgtgtccttactggataacttgtgtatttttcaagttaaaagatttcctaaaagtgcattacttgaatgtttatgtcatttatcacacatactttcaaaacataaccttcttcaaccagtatcttccttcctaaaatactgttaatagctggctattttgtaagcctgtgtctggttaaaaaaaagaaatgaatattttcaatacttatgaaccatagaattctgtattcttttcgtaacttttaccttttccataggacctatcaccatgtaaaatattatacatatttatttgtttattgtctacgtacctatactagaacatagtaaacatgagaacagggaatcttggttattaattcctgcagcactcagctacttatcactcaaacatttgttcttgaaaagaacttcaaataacatcattagatttttacagaatctttaaaaaagctttaatattttcaaggtttttttatttatataaacattgttacttcactactagtgattttaatagtggcattgtcacatgattcaaaaagaggaggccaatacattactgttgtattaagaagtcagttcaggcttaatatatgtgactactgtaggtcaacctcacattttaatgtatgaagaagattgtatgaacgatcacatcaattcatttaaatagtatgtattttgatactaattaagttttaagaagaaatgtatcattatcttgaaaataaaagataatttgaaaagcatgtcatgctatttatatcagggaaaatttgttcagctcacacaaagccatacattgacattttttttttaaactttgtggcactctatagtctttgaatttatttaatacctgttaagagatataacaaactgaaagttatttcaggagtgtttaatgtatttaatggaatgacgttgacgtactaagtaatagataaaatgttaagagctttctcggtgtcaggcactataacaagcattctttaggcattattttgtataatatgtacagccttatgaggtatgtactcttattgctattttcctttttacagtttaaaaactgaggcatagttcaa

SEQ ID NO: 4 - DNA, 2740 bp, Macaca fascicularis
  source 1..2740: mol_type = unassigned DNA
ctccgggagcactgtgcgcaggcgcgggagcaggttacggcggcgcttggcctctgctgatgccgtcgttgttctacccctcccccatcccagctctgcggcggccgcgcgctccaggccggtcgctccaccccccggctcccgggactgtggactccgtgaccctgtcctcggccctgtccgcgccgaagcagcccgggactgcgcagcgccccgcgtgccgacatgggaaagtctctttctcatttgcctttgcattcaagcaaagaagatgcttatgatggagtcacatctgaaaatatgagaaatggattggttaatagtgaagtccataatgaagatggaagaaatggagatgtctctcagtttccatatgtggaatttacaggaagagatagtgtcacctgccctacttgtcagggaacaggaagaattcctagggggcaagaaaaccagctggtggcattgattccatatagtgatcagagattaaggccaagaagaacaaagctgtatgtgatggcttctgtgtttgtctgtctgctcctttccggattggctgtgtttttccttttccctcgctctatcgacgtgaaatacattggtgtaaaatcagcatatgtcagttatgatgttcagaagcgtacaatttatttaaatatcacaaacacactaaatataacgaacaataactattattctgtcgaagttgaaaacatcactgcccaagttcaatttgcgaaaacagttattggaaaggcacgcttaaacaacataacccatattggtccacttgatatgaaacaaattgattacacagtacctaccgttatagcagaggaaatgagttatatgtatgatttctgtactctgatatccatcaaagtgcataacatagtactcatgatgcaagttactgtgacaacaacatactttggccactctgaacagatatcccaggagagatatcagtatgtcgactgtggaagaaacacaacttatcagttagggcagtctgaatatttaaatgtacttcagccacaacagtaaaaactggaagagatggatttaaagaagaaatatctattgatatttcctacactctcagtgaagaggtatttcctaataggagatcttaaattgaaccaacctaaagtttacacttctattttaagagtacagttaaaagtatgtggacctgcagttcttgtaactctccactctgtgttaatgatgtatttgtactaggatcttttacttgaatctaaatttactgtttgacttccttctccaacctatcccctacagggaaaagctgatacttcccctatagtacaataaataattatttaaaagtcatcgctccagtcactactgaaaacataattttggtgataaacataatttgagaaacttagtttctgaatgtttttatagaaaattactgaaagtctattattcatggaagacttttaaataaccttttttcctgttttataaattcccgttgttatatggtagtatttcagctacacaatattttagctttcagctaaacatttatagcttttcatttgttgacgctgtaatcatctgcatgtttttgttacttgcttcaagttagtgattgcctaacacttgtaagccaaaataatctttgcaaaattccatacctaaaattttgaaagctcctgatgttttcacatgtctttctgtattatagttttgtgaaatctttgtgtgatcttcaaacattatcatttaatgtacaatactgtaaataaactgtgcatggcttttgtacagctttagtaaatgtcaaataaagtggtacagattcattacaacaagtttctcgtaaaaatacaataaataggaaaacgaaattcagaaacccatagattgggaataggttccaattacatgttggatctggcataaaataaatttgaactattttgatgctccattttttttatgttgcttttcatgctagagaatggtgtatacatattttccaactgttaggtacccagttatcaattttatcaatgttttacagaaggaaacttttttttgatagatactgttcaagaaatcctgtcattaaatgatggtggctatatcaaaataaaacctatttagaaatttaatcactttgcacatcacttggaatatgatgcctctagtagttacttttttatagttttctacttttagttttaattttatttaaaattgtattcaaatatagattattgacttactttgctgttttatattttcaatatcatttttcatttaattttttttttcacttaccaagttctagggacatttaaaatatgtatcactaagtgtaggagtgatgataccaaaaaatgtaggtgggttaagattaatttcattctgttttctcgtgacagaaatcaggtttccctttccccatcccctaagtgcctaacttaggtctgaaacagcctgtttgtttattagtctgactctctcaagcataaaacgtaagctttatttgtttaattctgcctttaaacacactcaggtttccccttaattttcatattattttctccaagttttcctgagtgaatatcttaaattcgttgaatgtgctttttctttctttttacactagtcttcccttaattcattgctaactcaggccatccttactattaaacccaaatcagtcctttaagttcattatggcctttcctagtat

SEQ ID NO: 5 - DNA, 17 bp, synthetic construct
  source 1..17: mol_type = other DNA; note = Synthetic Construct
ataagtgttcattctcc

SEQ ID NO: 6 - DNA, 20 bp, synthetic construct
  source 1..20: mol_type = other DNA; note = Synthetic Construct
ttcatactattcttatttca

SEQ ID NO: 7 - DNA, 17 bp, synthetic construct
  source 1..17: mol_type = other DNA; note = Synthetic Construct
attatgtttatcaccaa

SEQ ID NO: 8 - DNA, 17 bp, synthetic construct
  source 1..17: mol_type = other DNA; note = Synthetic Construct
aacagtctttttactcc

SEQ ID NO: 9 - DNA, 20 bp, synthetic construct
  source 1..20: mol_type = other DNA; note = Synthetic Construct
tttcatactattcttatttc

SEQ ID NO: 10 - DNA, 18 bp, synthetic construct
  source 1..18: mol_type = other DNA; note = Synthetic Construct
catactattcttatttca

SEQ ID NO: 11 - DNA, 18 bp, synthetic construct
  source 1..18: mol_type = other DNA; note = Synthetic Construct
ttcttctttaaatccatc

SEQ ID NO: 12 - DNA, 18 bp, synthetic construct
  source 1..18: mol_type = other DNA; note = Synthetic Construct
gacatatacactcaaata

SEQ ID NO: 13 - DNA, 17 bp, synthetic construct
  source 1..17: mol_type = other DNA; note = Synthetic Construct
tgtttacatatctcacc

SEQ ID NO: 14 - DNA, 18 bp, synthetic construct
  source 1..18: mol_type = other DNA; note = Synthetic Construct
ttcttatttcaaatctca

SEQ ID NO: 15 - DNA, 17 bp, synthetic construct
  source 1..17: mol_type = other DNA; note = Synthetic Construct
tcttatttcaaatctca

SEQ ID NO: 16 - DNA, 18 bp, synthetic construct
  source 1..18: mol_type = other DNA; note = Synthetic Construct
atgtttatcaccaaaatt

SEQ ID NO: 17 - DNA, 17 bp, synthetic construct
  source 1..17: mol_type = other DNA; note = Synthetic Construct
tgtttacatatctcacc

SEQ ID NO: 18 - DNA, 20 bp, synthetic construct
  source 1..20: mol_type = other DNA; note = Synthetic Construct
ccaaatcttataataactac

SEQ ID NO: 19 - DNA, 18 bp, synthetic construct
  source 1..18: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid 5-methylcytosine phosphorothioate
  modified_base 4: mod_base = OTHER; note = thymidine phosphorothioate
  modified_base 5: mod_base = OTHER; note = thymidine phosphorothioate
  modified_base 6: mod_base = OTHER; note = cytidine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid 5-methylcytosine
ttcttctttaaatccatc

SEQ ID NO: 20 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = adenosine phosphorothioate
  modified_base 4: mod_base = OTHER; note = beta-D-oxy-locked nucleic acid adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
ataagtgttcattctcc

SEQ ID NO: 21 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid adenosine phosphorothioate
  modified_base 4: mod_base = OTHER; note = beta-D-oxy-locked nucleic acid adenosine phosphorothioate
  modified_base 5: mod_base = OTHER; note = guanosine phosphorothioate
  modified_base 6: mod_base = OTHER; note = 2'-O-methylthymidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
ataagtgttcattctcc

SEQ ID NO: 22 - DNA, 18 bp, synthetic construct
  source 1..18: mol_type = other DNA
  modified_base 1: mod_base = OTHER; note = beta-D-oxy-locked nucleic acid 5-methylcytosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid thymidine phosphorothioate
  modified_base 4: mod_base = OTHER; note = beta-D-oxy-locked nucleic acid adenosine phosphorothioate
  modified_base 5: mod_base = OTHER; note = cytidine phosphorothioate
  modified_base 6: mod_base = OTHER; note = thymidine phosphorothioate
  modified_base 7: mod_base = OTHER; note = adenosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = adenosine phosphorothioate
  modified_base 14: mod_base = OTHER; note = thymidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid thymidine phosphorothioate
  modified_base 17: mod_base = OTHER; note = beta-D-oxy-locked nucleic acid 5-methylcytosine phosphorothioate
  modified_base 18: mod_base = OTHER; note = beta-D-oxy-locked nucleic acid adenosine
catactattcttatttca

SEQ ID NO: 23 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid 5-methylcytosine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = adenosine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = thymidine phosphorothioate
  modified_base 14: mod_base = OTHER; note = thymidine phosphorothioate
  modified_base 15: mod_base = OTHER; note = adenosine phosphorothioate
  modified_base 16: mod_base = OTHER; note = thymidine phosphorothioate
  modified_base 17: mod_base = OTHER; note = beta-D-oxy-locked nucleic acid thymidine phosphorothioate
  modified_base 18: mod_base = OTHER; note = thymidine phosphorothioate
  modified_base 19: mod_base = OTHER; note = beta-D-oxy-locked nucleic acid 5-methylcytosine phosphorothioate
  modified_base 20: mod_base = OTHER; note = beta-D-oxy-locked nucleic acid adenosine
ttcatactattcttatttca

SEQ ID NO: 24 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = cytidine phosphorothioate
  modified_base 4: mod_base = OTHER; note = beta-D-oxy-locked nucleic acid adenosine phosphorothioate
  modified_base 5: mod_base = OTHER; note = beta-D-oxy-locked nucleic acid thymidine phosphorothioate
  modified_base 6: mod_base = OTHER; note = adenosine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = adenosine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = thymidine phosphorothioate
  modified_base 14: mod_base = OTHER; note = thymidine phosphorothioate
  modified_base 15: mod_base = OTHER; note = adenosine phosphorothioate
  modified_base 16: mod_base = OTHER; note = thymidine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid 5-methylcytosine phosphorothioate
  modified_base 20: mod_base = OTHER; note = beta-D-oxy-locked nucleic acid adenosine
ttcatactattcttatttca

SEQ ID NO: 25 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid 5-methylcytosine phosphorothioate
  modified_base 5: mod_base = OTHER; note = beta-D-oxy-locked nucleic acid adenosine phosphorothioate
  modified_base 6: mod_base = OTHER; note = thymidine phosphorothioate
  modified_base 7: mod_base = OTHER; note = beta-D-oxy-locked nucleic acid adenosine phosphorothioate
  modified_base 8: mod_base = OTHER; note = cytidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = thymidine phosphorothioate
  modified_base 15: mod_base = OTHER; note = thymidine phosphorothioate
  modified_base 16: mod_base = OTHER; note = adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid thymidine phosphorothioate
  modified_base 20: mod_base = OTHER; note = beta-D-oxy-locked nucleic acid 5-methylcytosine
tttcatactattcttatttc

SEQ ID NO: 26 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid adenosine phosphorothioate
  modified_base 4: mod_base = OTHER; note = adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine phosphorothioate
ataagtgttcattctcc

SEQ ID NO: 27 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = guanosine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = adenosine phosphorothioate
  modified_base 14: mod_base = OTHER; note = beta-D-oxy-locked nucleic acid 5-methylcytosine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid adenosine phosphorothioate
attatgtttatcaccaa

SEQ ID NO: 28 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid 5-methylcytosine phosphorothioate
  modified_base 4: mod_base = OTHER; note = adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = adenosine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
aacagtctttttactcc

SEQ ID NO: 29 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid thymidine phosphorothioate
  modified_base 4: mod_base = OTHER; note = beta-D-oxy-locked nucleic acid 5-methylcytosine phosphorothioate
  modified_base 5: mod_base = OTHER; note = adenosine phosphorothioate
  modified_base 6: mod_base = OTHER; note = thymidine phosphorothioate
  modified_base 7: mod_base = OTHER; note = adenosine phosphorothioate
  modified_base 8: mod_base = OTHER; note = cytidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = thymidine phosphorothioate
  modified_base 15: mod_base = OTHER; note = thymidine phosphorothioate
  modified_base 16: mod_base = OTHER; note = adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid thymidine phosphorothioate
  modified_base 20: mod_base = OTHER; note = beta-D-oxy-locked nucleic acid 5-methylcytosine
tttcatactattcttatttc

SEQ ID NO: 30 - DNA, 18 bp, synthetic construct
  source 1..18: mol_type = other DNA
  modified_base 1: mod_base = OTHER; note = beta-D-oxy-locked nucleic acid guanosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid 5-methylcytosine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = thymidine phosphorothioate
  modified_base 8: mod_base = OTHER; note = adenosine phosphorothioate
  modified_base 9: mod_base = OTHER; note = cytidine phosphorothioate
  modified_base 10: mod_base = OTHER; note = adenosine phosphorothioate
  modified_base 11: mod_base = OTHER; note = cytidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = adenosine phosphorothioate
  modified_base 15: mod_base = OTHER; note = beta-D-oxy-locked nucleic acid adenosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid adenosine
gacatatacactcaaata

SEQ ID NO: 31 - DNA, 18 bp, synthetic construct
  source 1..18: mol_type = other DNA
  modified_base 1: mod_base = OTHER; note = beta-D-oxy-locked nucleic acid guanosine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid thymidine phosphorothioate
  modified_base 5: mod_base = OTHER; note = adenosine phosphorothioate
  modified_base 6: mod_base = OTHER; note = thymidine phosphorothioate
  modified_base 7: mod_base = OTHER; note = cytidine phosphorothioate
  modified_base 8: mod_base = OTHER; note = adenosine phosphorothioate
  modified_base 9: mod_base = OTHER; note = cytidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = adenosine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid thymidine phosphorothioate
  modified_base 17: mod_base = OTHER; note = beta-D-oxy-locked nucleic acid adenosine phosphorothioate
  modified_base 18: mod_base = OTHER; note = beta-D-oxy-locked nucleic acid thymidine
gtttatcaccaaaattat

SEQ ID NO: 32 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid 5-methylcytosine phosphorothioate
  modified_base 4: mod_base = OTHER; note = adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = adenosine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
aacagtctttttactcc

SEQ ID NO: 33 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid adenosine phosphorothioate
  modified_base 4: mod_base = OTHER; note = adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
ataagtgttcattctcc

SEQ ID NO: 34 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = adenosine phosphorothioate
  modified_base 4: mod_base = OTHER; note = beta-D-oxy-locked nucleic acid adenosine phosphorothioate
  modified_base 5: mod_base = OTHER; note = guanosine phosphorothioate
  modified_base 6: mod_base = OTHER; note = thymidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
ataagtgttcattctcc

SEQ ID NO: 35 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid adenosine phosphorothioate
  modified_base 4: mod_base = OTHER; note = beta-D-oxy-locked nucleic acid adenosine phosphorothioate
  modified_base 5: mod_base = OTHER; note = guanosine phosphorothioate
  modified_base 6: mod_base = OTHER; note = 2?-O-methyluridine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = thymidine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
  misc_feature 1..5: note = DNA
  misc_feature 6: note = RNA
  misc_feature 7..17: note = DNA
ataagtgttcattctcc

SEQ ID NO: 36 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = guanosine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = adenosine phosphorothioate
  modified_base 14: mod_base = OTHER; note = beta-D-oxy-locked nucleic acid 5-methylcytosine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid adenosine
  modified_base 17: mod_base = OTHER; note = inverted adenosine
attatgtttatcaccaa

SEQ ID NO: 37 - DNA, 17 bp, synthetic construct
  source 1..17: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = adenosine phosphorothioate
  modified_base 5: mod_base = OTHER; note = thymidine phosphorothioate
  modified_base 6: mod_base = OTHER; note = guanosine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = adenosine phosphorothioate
  modified_base 14: mod_base = OTHER; note = cytidine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid adenosine
attatgtttatcaccaa

SEQ ID NO: 38 - DNA, 18 bp, synthetic construct
  source 1..18: mol_type = other DNA
  modified_base 1: mod_base = OTHER; note = beta-D-oxy-locked nucleic acid 5-methylcytosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid thymidine phosphorothioate
  modified_base 4: mod_base = OTHER; note = beta-D-oxy-locked nucleic acid adenosine phosphorothioate
  modified_base 5: mod_base = OTHER; note = cytidine phosphorothioate
  modified_base 6: mod_base = OTHER; note = thymidine phosphorothioate
  modified_base 7: mod_base = OTHER; note = adenosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = adenosine phosphorothioate
  modified_base 14: mod_base = OTHER; note = thymidine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid thymidine phosphorothioate
  modified_base 17: mod_base = OTHER; note = beta-D-oxy-locked nucleic acid 5-methylcytosine phosphorothioate
  modified_base 18: mod_base = OTHER; note = beta-D-oxy-locked nucleic acid adenosine
catactattcttatttca

SEQ ID NO: 39 - DNA, 20 bp, synthetic construct
  source 1..20: mol_type = other DNA
  modified_base 1: mod_base = OTHER; note = beta-D-oxy-locked nucleic acid 5-methylcytosine phosphorothioate
  modified_base 2: mod_base = OTHER; note = cytidine phosphorothioate
  modified_base 3: mod_base = OTHER; note = beta-D-oxy-locked nucleic acid adenosine phosphorothioate
  modified_base 4: mod_base = OTHER; note = beta-D-oxy-locked nucleic acid adenosine phosphorothioate
  modified_base 5: mod_base = OTHER; note = beta-D-oxy-locked nucleic acid adenosine phosphorothioate
  modified_base 6: mod_base = OTHER; note = thymidine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = thymidine phosphorothioate
  modified_base 15: mod_base = OTHER; note = adenosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid 5-methylcytosine phosphorothioate
  modified_base 18: mod_base = OTHER; note = thymidine phosphorothioate
  modified_base 19: mod_base = OTHER; note = beta-D-oxy-locked nucleic acid adenosine phosphorothioate
  modified_base 20: mod_base = OTHER; note = beta-D-oxy-locked nucleic acid 5-methylcytosine
ccaaatcttataataactac

SEQ ID NO: 40 - DNA, 18 bp, synthetic construct
  source 1..18: mol_type = other DNA
  modified_base 1: mod_base = OTHER; note = beta-D-oxy-locked nucleic acid guanosine phosphorothioate
  modified_base 2: mod_base = OTHER; note = beta-D-oxy-locked nucleic acid adenosine phosphorothioate
  modified_base 3: mod_base = OTHER; note = beta-D-oxy-locked nucleic acid 5-methylcytosine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = thymidine phosphorothioate
  modified_base 8: mod_base = OTHER; note = adenosine phosphorothioate
  modified_base 9: mod_base = OTHER; note = cytidine phosphorothioate
  modified_base 10: mod_base = OTHER; note = adenosine phosphorothioate
  modified_base 11: mod_base = OTHER; note = cytidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = adenosine phosphorothioate
  modified_base 15: mod_base = OTHER; note = beta-D-oxy-locked nucleic acid adenosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid adenosine
gacatatacactcaaata

SEQ ID NO: 41 - DNA, 17 bp, synthetic construct
  source 1..17: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid 5-methylcytosine phosphorothioate
  modified_base 3: mod_base = OTHER; note = beta-D-oxy-locked nucleic acid thymidine phosphorothioate
  modified_base 4: mod_base = OTHER; note = beta-D-oxy-locked nucleic acid thymidine phosphorothioate
  modified_base 5: mod_base = OTHER; note = adenosine phosphorothioate
  modified_base 6: mod_base = OTHER; note = thymidine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = cytidine phosphorothioate
  modified_base 10: mod_base = OTHER; note = adenosine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = thymidine phosphorothioate
  modified_base 14: mod_base = OTHER; note = beta-D-oxy-locked nucleic acid 5-methylcytosine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid adenosine
tcttatttcaaatctca

SEQ ID NO: 42 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = cytidine phosphorothioate
  modified_base 4: mod_base = OTHER; note = beta-D-oxy-locked nucleic acid adenosine phosphorothioate
  modified_base 5: mod_base = OTHER; note = beta-D-oxy-locked nucleic acid thymidine phosphorothioate
  modified_base 6: mod_base = OTHER; note = beta-D-oxy-locked nucleic acid adenosine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = adenosine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = thymidine phosphorothioate
  modified_base 14: mod_base = OTHER; note = thymidine phosphorothioate
  modified_base 15: mod_base = OTHER; note = adenosine phosphorothioate
  modified_base 16: mod_base = OTHER; note = thymidine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid 5-methylcytosine phosphorothioate
  modified_base 20: mod_base = OTHER; note = beta-D-oxy-locked nucleic acid adenosine
ttcatactattcttatttca

SEQ ID NO: 43 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid 5-methylcytosine phosphorothioate
  modified_base 4: mod_base = OTHER; note = beta-D-oxy-locked nucleic acid adenosine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = cytidine phosphorothioate
  modified_base 8: mod_base = OTHER; note = thymidine phosphorothioate
  modified_base 9: mod_base = OTHER; note = adenosine phosphorothioate
  modified_base 10: mod_base = OTHER; note = thymidine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = thymidine phosphorothioate
  modified_base 14: mod_base = OTHER; note = thymidine phosphorothioate
  modified_base 15: mod_base = OTHER; note = adenosine phosphorothioate
  modified_base 16: mod_base = OTHER; note = thymidine phosphorothioate
  modified_base 17: mod_base = OTHER; note = beta-D-oxy-locked nucleic acid thymidine phosphorothioate
  modified_base 18: mod_base = OTHER; note = thymidine phosphorothioate
  modified_base 19: mod_base = OTHER; note = beta-D-oxy-locked nucleic acid 5-methylcytosine phosphorothioate
  modified_base 20: mod_base = OTHER; note = beta-D-oxy-locked nucleic acid adenosine
ttcatactattcttatttca

SEQ ID NO: 44 - DNA, 18 bp, synthetic construct
  source 1..18: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid 5-methylcytosine phosphorothioate
  modified_base 4: mod_base = OTHER; note = beta-D-oxy-locked nucleic acid thymidine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = thymidine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid thymidine phosphorothioate
  modified_base 17: mod_base = OTHER; note = beta-D-oxy-locked nucleic acid 5-methylcytosine phosphorothioate
  modified_base 18: mod_base = OTHER; note = beta-D-oxy-locked nucleic acid adenosine
ttcttatttcaaatctca

SEQ ID NO: 45 - DNA, 18 bp, synthetic construct
  source 1..18: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid 5-methylcytosine phosphorothioate
  modified_base 4: mod_base = OTHER; note = thymidine phosphorothioate
  modified_base 5: mod_base = OTHER; note = thymidine phosphorothioate
  modified_base 6: mod_base = OTHER; note = cytidine phosphorothioate
  modified_base 7: mod_base = OTHER; note = thymidine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = thymidine phosphorothioate
  modified_base 14: mod_base = OTHER; note = cytidine phosphorothioate
  modified_base 15: mod_base = OTHER; note = beta-D-oxy-locked nucleic acid 5-methylcytosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid 5-methylcytosine
ttcttctttaaatccatc

SEQ ID NO: 46 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid thymidine phosphorothioate
  modified_base 4: mod_base = OTHER; note = beta-D-oxy-locked nucleic acid 5-methylcytosine phosphorothioate
  modified_base 5: mod_base = OTHER; note = adenosine phosphorothioate
  modified_base 6: mod_base = OTHER; note = thymidine phosphorothioate
  modified_base 7: mod_base = OTHER; note = adenosine phosphorothioate
  modified_base 8: mod_base = OTHER; note = cytidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = thymidine phosphorothioate
  modified_base 15: mod_base = OTHER; note = thymidine phosphorothioate
  modified_base 16: mod_base = OTHER; note = adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid thymidine phosphorothioate
  modified_base 20: mod_base = OTHER; note = beta-D-oxy-locked nucleic acid 5-methylcytosine
tttcatactattcttatttc

SEQ ID NO: 47 - DNA, 20 bp, synthetic construct
  source 1..20: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid 5-methylcytosine phosphorothioate
  modified_base 5: mod_base = OTHER; note = beta-D-oxy-locked nucleic acid adenosine phosphorothioate
  modified_base 6: mod_base = OTHER; note = thymidine phosphorothioate
  modified_base 7: mod_base = OTHER; note = beta-D-oxy-locked nucleic acid adenosine phosphorothioate
  modified_base 8: mod_base = OTHER; note = cytidine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = thymidine phosphorothioate
  modified_base 13: mod_base = OTHER; note = cytidine phosphorothioate
  modified_base 14: mod_base = OTHER; note = thymidine phosphorothioate
  modified_base 15: mod_base = OTHER; note = thymidine phosphorothioate
  modified_base 16: mod_base = OTHER; note = adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine phosphorothioate
  modified_base 19: mod_base = OTHER; note = beta-D-oxy-locked nucleic acid thymidine phosphorothioate
  modified_base 20: mod_base = OTHER; note = beta-D-oxy-locked nucleic acid 5-methylcytosine
tttcatactattcttatttc

SEQ ID NO: 48 - DNA, 18 bp, synthetic construct
  source 1..18: mol_type = other DNA
  modified_base 1: mod_base = OTHER; note = beta-D-oxy-locked nucleic acid guanosine phosphorothioate
  modified_base 2: mod_base = OTHER; note = beta-D-oxy-locked nucleic acid thymidine phosphorothioate
  modified_base 3: mod_base = OTHER; note = thymidine phosphorothioate
  modified_base 4: mod_base = OTHER; note = beta-D-oxy-locked nucleic acid thymidine phosphorothioate
  modified_base 5: mod_base = OTHER; note = adenosine phosphorothioate
  modified_base 6: mod_base = OTHER; note = thymidine phosphorothioate
  modified_base 7: mod_base = OTHER; note = cytidine phosphorothioate
  modified_base 8: mod_base = OTHER; note = adenosine phosphorothioate
  modified_base 9: mod_base = OTHER; note = cytidine phosphorothioate
  modified_base 10: mod_base = OTHER; note = cytidine phosphorothioate
  modified_base 11: mod_base = OTHER; note = adenosine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = adenosine phosphorothioate
  modified_base 15: mod_base = OTHER; note = beta-D-oxy-locked nucleic acid thymidine phosphorothioate
  modified_base 16: mod_base = OTHER; note = beta-D-oxy-locked nucleic acid thymidine phosphorothioate
  modified_base 17: mod_base = OTHER; note = beta-D-oxy-locked nucleic acid adenosine phosphorothioate
  modified_base 18: mod_base = OTHER; note = beta-D-oxy-locked nucleic acid thymidine
gtttatcaccaaaattat

SEQ ID NO: 49 - DNA, 18 bp, synthetic construct
  source 1..18: mol_type = other DNA
  modified_base 1: mod_base = OTHER; note = beta-D-oxy-locked nucleic acid adenosine phosphorothioate
  modified_base 2: mod_base = OTHER; note = beta-D-oxy-locked nucleic acid thymidine phosphorothioate
  modified_base 3: mod_base = OTHER; note = beta-D-oxy-locked nucleic acid guanosine phosphorothioate
  modified_base 4: mod_base = OTHER; note = beta-D-oxy-locked nucleic acid thymidine phosphorothioate
  modified_base 5: mod_base = OTHER; note = thymidine phosphorothioate
  modified_base 6: mod_base = OTHER; note = thymidine phosphorothioate
  modified_base 7: mod_base = OTHER; note = adenosine phosphorothioate
  modified_base 8: mod_base = OTHER; note = thymidine phosphorothioate
  modified_base 9: mod_base = OTHER; note = cytidine phosphorothioate
  modified_base 10: mod_base = OTHER; note = adenosine phosphorothioate
  modified_base 11: mod_base = OTHER; note = cytidine phosphorothioate
  modified_base 12: mod_base = OTHER; note = cytidine phosphorothioate
  modified_base 13: mod_base = OTHER; note = adenosine phosphorothioate
  modified_base 14: mod_base = OTHER; note = adenosine phosphorothioate
  modified_base 15: mod_base = OTHER; note = beta-D-oxy-locked nucleic acid adenosine phosphorothioate
  modified_base 16: mod_base = OTHER; note = beta-D-oxy-locked nucleic acid adenosine phosphorothioate
  modified_base 17: mod_base = OTHER; note = beta-D-oxy-locked nucleic acid thymidine phosphorothioate
  modified_base 18: mod_base = OTHER; note = beta-D-oxy-locked nucleic acid thymidine
atgtttatcaccaaaatt

SEQ ID NO: 50 - DNA, 17 bp, synthetic construct
  source 1..17: mol_type = other DNA
  modified_base 1: mod_base = OTHER; note = beta-D-oxy-locked nucleic acid thymidine phosphorothioate
  modified_base 2: mod_base = OTHER; note = beta-D-oxy-locked nucleic acid guanosine phosphorothioate
  modified_base 3: mod_base = OTHER; note = thymidine phosphorothioate
  modified_base 4: mod_base = OTHER; note = thymidine phosphorothioate
  modified_base 5: mod_base = OTHER; note = thymidine phosphorothioate
  modified_base 6: mod_base = OTHER; note = adenosine phosphorothioate
  modified_base 7: mod_base = OTHER; note = cytidine phosphorothioate
  modified_base 8: mod_base = OTHER; note = adenosine phosphorothioate
  modified_base 9: mod_base = OTHER; note = thymidine phosphorothioate
  modified_base 10: mod_base = OTHER; note = adenosine phosphorothioate
  modified_base 11: mod_base = OTHER; note = thymidine phosphorothioate
  modified_base 12: mod_base = OTHER; note = cytidine phosphorothioate
  modified_base 13: mod_base = OTHER; note = thymidine phosphorothioate
  modified_base 14: mod_base = OTHER; note = beta-D-oxy-locked nucleic acid 5-methylcytosine phosphorothioate
  modified_base 15: mod_base = OTHER; note = beta-D-oxy-locked nucleic acid adenosine phosphorothioate
  modified_base 16: mod_base = OTHER; note = beta-D-oxy-locked nucleic acid 5-methylcytosine phosphorothioate
  modified_base 17: mod_base = OTHER; note = beta-D-oxy-locked nucleic acid 5-methylcytosine
tgtttacatatctcacc

SEQ ID NO: 51 - DNA, 21 bp, synthetic construct
  source 1..21: mol_type = other DNA
tcccacttatgagtggcaact

(19) United States
(12) Patent Application Publication
(10) Pub. No.: US 2024/0376474 A1
(43) Pub. Date: Nov. 14, 2024

(54) ANTISENSE OLIGONUCLEOTIDES FOR MODULATING TMEM106B EXPRESSION

(71) Applicant: Genentech, Inc., South San Francisco, CA (US)

(72) Inventors: Amy EASTON, South San Francisco, CA (US); Helene GYLLING, Copenhagen (DK); Dennis HANSEN, Farum (DK); Lukasz KIELPINSKI, Hørsholm (DK); Denia MELLAL, Saint Louis (FR); Helle NYMARK, Farum (DK); Benny CHIH, South San Francisco, CA (US); Katarzyna CHYZYNSKA, Charlottenlund (DK)

(21) Appl. No.: 18/777,933

(22) Filed: Jul. 19, 2024

Related U.S. Application Data
(63) Continuation of application No. PCT/US2023/060907, filed on Jan. 19, 2023.
(60) Provisional application No. 63/301,348, filed on Jan. 20, 2022.

(51) Int. Cl. C12N 15/113

(52) U.S. Cl. CPC C12N 15/113; C12N 2310/11; C12N 2310/315; C12N 2310/321; C12N 2310/3231; C12N 2310/3341

(57) ABSTRACT

The present disclosure relates to antisense oligonucleotides that are capable of modulating expression of TMEM106B in a target cell. The antisense oligonucleotides hybridize to TMEM106B mRNA. The present disclosure further relates to methods of using the antisense oligonucleotides described herein.

### SEQUENCE LISTING

The instant application contains a Sequence Listing which has been submitted electronically in XML format and is hereby incorporated by reference in its entirety. Said XML copy, created on Jul. 12, 2024, is named 51527-003002_Sequence_Listing_7_12_24.xml and is 450,085 bytes in size.

### FIELD OF DISCLOSURE

The present disclosure relates to antisense oligonucleotides that are complementary to Transmembrane Protein 106B (TMEM106B) messenger RNA (mRNA) and capable of inhibiting the expression of TMEM106B protein. Modulation of TMEM106B expression is beneficial for a range of medical disorders, such as neurological disorders, e.g., neurodegenerative disorders, such as, e.g., frontotemporal lobar degeneration.

### BACKGROUND OF THE DISCLOSURE

Transmembrane Protein 106B (TMEM106B) is a single-pass, type 2 integral membrane glycoprotein located predominantly in the membranes of endosomes and lysosomes. TMEM106B is expressed in neurons, glia, and endothelial cells. It is believed to be involved in dendrite morphogenesis, such as dendrite branching, as well as lysosomal function. Several common neurodegenerative disorders, including frontotemporal lobar degeneration (FTLD), have been associated with dysregulated TMEM106B expression. Neurodegenerative disorders represent a major class of neurological conditions for which there is a dearth of curative therapies. Therefore, there is a need for of agents that modulate TMEM106B expression for use in therapeutic applications.

### SUMMARY OF DISCLOSURE

The present disclosure provides antisense oligonucleotides (ASOs) which reduce TMEM106B expression in vitro and in vivo. The disclosure identified specific ASO compounds that target regions in the human TMEM106B pre-mRNA which robustly inhibit TMEM106B expression. The disclosure also provides ASO sequences, modification motifs, compounds, conjugates, and salts which are capable of reducing TMEM106B expression. Also provided by the present disclosure are methods of treatment of diseases or disorders characterized by neurodegeneration, such as, e.g., frontotemporal lobar degeneration (FTLD), Parkinson's disease (or parkinsonism), hypomyelinating leukodystrophies, amyotrophic lateral sclerosis (ALS), multiple system atrophy (MSA), Alzheimer's disease (AD), motor neuron disease (MND), corticobasal syndrome (CBS), progressive supranuclear palsy (PSP), and neuronal ceroid lipofuscinosis (NCL). Such disorders may be characterized by dysregulated (e.g., increased) TMEM106B expression or activity.

In an aspect, the disclosure provides an antisense oligonucleotide, wherein the antisense oligonucleotide is a compound selected from the group consisting of:

(SEQ ID NO: 19; CMP ID NO: 26)
TTCttctttaaatcCATC;
(SEQ ID NO: 20; CMP ID NO: 8_3)
ATaAgtgttcattcTCC;
(SEQ ID NO: 21; CMP ID NO: 8_4)
ATAAgTgttcattctCC;
(SEQ ID NO: 22; CMP ID NO: 12)
CATActattcttatTTCA;
(SEQ ID NO: 23; CMP ID NO: 24_3)
TTCAtactattcttatTtCA;
(SEQ ID NO: 24; CMP ID NO: 24_1)
TTCATactattcttatTTCA
(SEQ ID NO: 25; CMP ID NO: 29_5)
TTtCAtActattcttaTTTC;
(SEQ ID NO: 26; CMP ID NO: 8_1)
ATAagtgttcattcTCC;
(SEQ ID NO: 27; CMP ID NO: 10_4)
ATtAtgtttatcaCCAa;
(SEQ ID NO: 28; CMP ID NO: 6)
AACagtctttttacTCC;
(SEQ ID NO: 29; CMP ID NO: 29_3)
TTTCatactattcttaTTTC;
(SEQ ID NO: 30; CMP ID NO: 14)
GACAtatacactcaAATA;
and
(SEQ ID NO: 31; CMP ID NO: 15)
GTtTatcaccaaaaTTAT,

wherein non-italicized capital letters are beta-D-oxy LNA nucleosides, italicized capital letters are 2′-O-methyl nucleosides, lowercase letters are DNA nucleosides, all LNA C are 5-methylcytosine, and all internucleoside linkages are phosphorothioate internucleoside linkages. In some embodiments, the antisense oligonucleotide is CMP ID NO: 26. In some embodiments, the antisense oligonucleotide is CMP ID NO: 8_3. In some embodiments, the antisense oligonucleotide is CMP ID NO: 8_4. In some embodiments, the antisense oligonucleotide is CMP ID NO: 12. In some embodiments, the antisense oligonucleotide is CMP ID NO: 24_3. In some embodiments, the antisense oligonucleotide is CMP ID NO: 24_1. In some embodiments, the antisense oligonucleotide is CMP ID NO: 29_5. In some embodiments, the antisense oligonucleotide is CMP ID NO: 8_1. In some embodiments, the antisense oligonucleotide is CMP ID NO: 10_4. In some embodiments, the antisense oligonucleotide is CMP ID NO: 6. In some embodiments, the antisense oligonucleotide is CMP ID NO: 29_3. In some embodiments, the antisense oligonucleotide is CMP ID NO: 14. In some embodiments, the antisense oligonucleotide is CMP ID NO: 15.

In some embodiments, the TMEM106B target nucleic acid is a mammalian TMEM106B target nucleic acid. In some embodiments, the mammalian TMEM106B target nucleic acid is a human TMEM106B target nucleic acid.

In another aspect, the disclosure provides a conjugate including any one of the antisense oligonucleotides described herein, and at least one conjugate moiety covalently attached to said antisense oligonucleotide.

In another aspect, the disclosure provides a pharmaceutically acceptable salt of any one of the antisense oligonucleotides described herein. In another aspect, the disclosure provides a pharmaceutically acceptable salt of any one of the conjugates described herein. In another aspect, the disclosure provides a pharmaceutical composition including any one of the antisense oligonucleotides described herein and a pharmaceutically acceptable diluent, solvent, carrier, salt or adjuvant. In another aspect, the disclosure provides a pharmaceutical composition including any one of the conjugates described herein and a pharmaceutically acceptable diluent, solvent, carrier, salt or adjuvant.

In another aspect, the disclosure provides an in vivo or in vitro method for modulating TMEM106B expression in a target cell which is expressing TMEM106B including administering any one of the pharmaceutical compositions described herein in an effective amount to said cell.

In another aspect, the disclosure provides a method for treating or preventing a disease including administering a therapeutically or prophylactically effective amount of any one of the pharmaceutical compositions described herein to a subject suffering from or susceptible to the disease.

In another aspect, the disclosure provides the pharmaceutical compositions described herein for use in the treatment or prevention of a disease.

### Definitions

The terms “antisense oligonucleotide” and “ASO,” as used herein, is defined as a molecule comprising two or more covalently linked nucleosides. Such covalently bound nucleosides may also be referred to as nucleic acid molecules or oligomers. ASOs are commonly made in the laboratory by solid-phase chemical synthesis followed by purification and isolation. When referring to a sequence of the ASO, reference is made to the sequence or order of nucleobase moieties, or modifications thereof, of the covalently linked nucleotides or nucleosides. The ASO of the disclosure is chemically synthesized and is typically purified or isolated. The ASOs of the disclosure may comprise one or more modified nucleosides or nucleotides, such as 2′ sugar-modified nucleosides or inverted nucleosides, among others. ASOs are capable of hybridizing to a target polynucleotide, such as an mRNA molecule (e.g., TMEM106B mRNA), and promoting the degradation of the polynucleotide through RNA interference. The ASOs of the disclosure are capable of modulating expression of a target gene by hybridizing to a target nucleic acid, in particular to a contiguous sequence on a target nucleic acid. The ASOs are not essentially double stranded and are therefore not siRNAs or shRNAs. The ASOs of the present disclosure are single-stranded. It is understood that single-stranded ASOs of the present disclosure can form hairpins or intermolecular duplex structures (duplex between two molecules of the same ASO), as long as the degree of complementarity is less than 50% across of the full length of the ASO. Advantageously, the single-stranded ASOs of the disclosure do not contain RNA nucleosides as incorporation of RNA nucleotides would decrease nuclease resistance.

Advantageously, the ASOs of the disclosure comprise one or more modified nucleosides or nucleotides, such as 2′ sugar-modified nucleosides. Furthermore, it is advantageous that the nucleosides which are not modified are DNA nucleosides.

As used herein, the term “contiguous nucleotide sequence” refers to the region of the ASO which is complementary to the TMEM106B target nucleic acid or target sequence. The term is used interchangeably herein with the term “contiguous nucleobase sequence” and “antisense oligonucleotide motif sequence.” All of the nucleotides of the ASO may constitute the contiguous nucleotide sequence. The ASO may include the contiguous nucleotide sequence, such as a F-G-F′ gapmer region described herein, and may, optionally, include additional nucleotides, such as, e.g., a nucleotide linker region which may be used to attach a functional group to the contiguous nucleotide sequence. The nucleotide linker region may or may not be complementary to the TMEM106B target nucleic acid. It is understood that the contiguous nucleotide sequence of the ASO cannot be longer than the ASO and that the ASO cannot be shorter than the contiguous nucleotide sequence.

Nucleotides are the building blocks of ASOs and polynucleotides, and for the purposes of the present disclosure include both naturally-occurring and non-naturally-occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers.”

The terms “modified nucleoside” or “nucleoside modification,” as used herein, refer to nucleosides modified as compared to an equivalent DNA or RNA nucleoside by the introduction of one or more modifications of the sugar moiety or the nucleobase. In some cases, the modified nucleoside includes a modified sugar moiety. The term “modified nucleoside” may also be used herein interchangeably with the term “nucleoside analogue” or modified “units” or modified “monomers.”

The term “modified internucleoside linkage” is defined as a linkage other than phosphodiester (PO) linkage, that covalently couples two nucleosides together. The ASOs of the disclosure may therefore include modified internucleoside linkages. Without wishing to be bound by theory, the modified internucleoside linkage may increase nuclease resistance of the ASO as compared to a phosphodiester linkage. For naturally-occurring ASOs, the internucleoside linkage includes phosphate groups creating a phosphodiester bond between adjacent nucleosides. Modified internucleoside linkages are particularly useful in stabilizing ASOs for in vivo use, and may serve to protect against nuclease cleavage at regions of DNA or RNA nucleosides in the ASO of the disclosure, such as, e.g., within the gap region G of a gapmer ASO, as well as in regions of modified nucleosides, such as, e.g., regions F and F′.

The ASOs of the disclosure may include one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) internucleoside linkages modified from the natural phosphodiester, such as one or more modified internucleoside linkages that are for example more resistant to nuclease attack (e.g., a phosphorothioate linkage). Nuclease resistance may be determined by incubating the ASO in blood serum or by using a nuclease resistance assay (e.g., snake venom phosphodiesterase (SVPD)), both are well known in the art. Internucleoside linkages which are capable of enhancing the nuclease resistance of an ASO are referred to as “nuclease-resistant internucleoside linkages.” The ASO agents of the disclosure may include at least 50% (e.g., at least 51%, 55%, 60%, 65%, 70%, 75%, 80%, 85%, 90%, 91%, 92%, 93%, 94%, 95%, 96%, 97%, 98%, 99% or more) of modified internucleoside linkages with respect to the total number of internucleoside linkages within the ASO. In some cases, all of the internucleoside linkages of the ASO or a contiguous nucleotide sequence thereof are modified. It will be recognized that, the nucleosides which link the ASO of the disclosure to a non-nucleotide functional group, such as a conjugate, may be phosphodiester. In some cases, all of the internucleoside linkages of the ASO or contiguous nucleotide sequence thereof are nuclease resistant internucleoside linkages.

Modified internucleoside linkages may be selected from the group including phosphorothioate, diphosphorothioate, and boranophosphate. In some embodiments, the modified internucleoside linkages are compatible with RNase H recruitment to the ASO. The internucleoside linkages may include a sulphur(S) atom, as is the case in a phosphorothioate internucleoside linkage. Phosphorothioate internucleoside linkages are particularly useful due to their ability to impart nuclease resistance, beneficial pharmacokinetics, and ease of manufacture. The ASOs disclosed herein may include at least 50% (e.g., at least 51%, 55%, 60%, 65%, 70%, 75%, 80%, 85%, 90%, 91%, 92%, 93%, 94%, 95%, 96%, 97%, 98%, 99% or more) of phosphorothioate linkages. The ASOs of the disclosure may also include internucleoside linkages which are all phosphorothioate internucleoside linkages. In some cases, the ASOs of the disclosure include phosphorothioate internucleoside linkages and at least one phosphodiester linkage, such as, e.g., 2, 3, or 4 phosphodiester linkages. Phosphodiester linkages in a gapmer oligonucleotide, when present, are suitably not located between contiguous DNA nucleosides in the gap region G.

In some embodiments, the ASO includes one or more neutral internucleoside linkages, such as an internucleoside linkage selected from phosphotriester, methylphosphonate, MMI, amide-3, formacetal or thioformacetal. Further internucleoside linkages are disclosed in WO2009/124238 (incorporated herein by reference). In some cases, the internucleoside linkage is selected from linkers disclosed in WO2007/031091 (incorporated herein by reference). Particularly, the internucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, and —NR^{H}—CO—NR^{H}—, or the internucleoside linker may be selected form the group consisting of: —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, and —CH₂—NCH₃—O—CH₂—, where R^{H} is selected from hydrogen and C1-4-alkyl.

Nuclease resistant linkages, such as phosphorothioate linkages, are particularly useful in ASO regions capable of recruiting nuclease when forming a duplex with the TMEM106B target nucleic acid, such as region G for gapmers. Phosphorothioate linkages may also be useful in non-nuclease recruiting regions or affinity-enhancing regions of the ASO, such as regions F and F′ in gapmers. Gapmer ASOs may include one or more phosphodiester linkages in region F or F′, or both region F and F′, where all the internucleoside linkages in region G may be phosphorothioate.

In some embodiments, the ASO of the disclosure includes a phosphodiester inverted internucleoside linkage. The term “inverted nucleoside” or “reverse nucleoside” or “DNA X^{INV}” (X=A, T, C, G) refers to a nucleoside which includes the dimethoxytrityl (DMT) and phosphoramidite groups reversed from the normal case; the DMT-group is attached to the 3′-OH, and the phosphoramidite attached to the 5′-OH of the ribose. Synthesis of inverted oligonucleosides are described in the Materials and Methods section herein.

The term “nucleobase,” as used herein, refers to purine (e.g., adenine and guanine) and pyrimidine (e.g., uracil, thymine, and cytosine) moieties present in nucleosides and nucleotides, which form hydrogen bonds during nucleic acid hybridization. In the context of the present disclosure, the term nucleobase also encompasses modified nucleobases which may differ from naturally-occurring nucleobases but are functional during nucleic acid hybridization. In this context, “nucleobase” refers to both naturally-occurring nucleobases, such as adenine, guanine, cytosine, thymidine, uracil, xanthine and hypoxanthine, as well as non-naturally-occurring variants. Such variants are for example described in Hirao et al., Accounts of Chemical Research 45:2055 (2012) and Bergstrom, Current Protocols in Nucleic Acid Chemistry Suppl. 37 1.4.1 (2009).

The nucleobase moiety may be modified by changing the purine or pyrimidine into a modified purine or pyrimidine, such as, e.g., a substituted purine or substituted pyrimidine, e.g., a nucleobase selected from 5-methylcytosine, 5-hydroxycytosine, 5-methoxycytosine, N⁴-methylcytosine, N³-Methylcytosine, N⁴-ethylcytosine, pseudoisocytosine, 5-fluorocytosine, 5-bromocytosine, 5-iodocytosine, 5-aminocytosine, 5-ethynylcytosine, 5-propynylcytosine, pyrrolocytosine, 5-aminomethylcytosine, 5-hydroxymethylcytosine, naphthyridine, 5-methoxyuracil, pseudouracil, dihydrouracil, 2-thiouracil, 4-thiouracil, 2-thiothymine, 4-thiothymine, 5,6-dihydrothymine, 5-halouracil, 5-propynyluracil, 5-aminomethyluracil, 5-hydroxymethyluracil, hypoxanthine, 7-deazaguanine, 8-aza-7-deazaguanine, 7-aza-2,6-diaminopurine, thienoguanine, N¹-methylguanine, N²-methylguanine, 6-thioguanine, 8-methoxyguanine, 8-allyloxyguanine, 7-aminomethyl-7-deazaguanine, 7-methylguanine, imidazopyridopyrimidine, 7-deazaadenine, 3-deazaadenine, 8-aza-7-deazaadenine, 8-aza-7-deazaadenine, N¹-methyladenine, 2-methyladenine, N⁶-methyladenine, 7-methyladenine, 8-methyladenine, or 8-azidoadenine.

The nucleobase moieties may be indicated by the letter code for each corresponding nucleobase, e.g., A, T, G, C, or U, where each letter may optionally include modified nucleobases of equivalent function (i.e., complementarity). For example, in the exemplified ASOs, the nucleobase moieties are selected from A, T, G, C, and 5-methylcytosine. Optionally, for LNA gapmers, 5-methylcytosine LNA nucleosides may be used.

The term “modified antisense oligonucleotide” describes an ASO including one or more sugar-modified nucleosides, modified nucleobases, or modified internucleoside linkages. The term “chimeric ASO” has been used in the literature to describe ASOs with modified nucleosides.

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that ASOs may include nucleosides with modified nucleobases. For example, 5-methylcytosine is often used in place of cytosine, and as such, the term “complementarity” encompasses Watson Crick base-paring between non-modified and modified nucleobases.

The term “% complementary,” as used herein, refers to the proportion of nucleotides of a contiguous nucleotide sequence in a nucleic acid molecule (e.g. ASO of the disclosure) which are complementary to a reference sequence (e.g. a target sequence or sequence motif). The percentage of complementarity is calculated by counting the number of aligned nucleobases that are complementary between the two sequences (when aligned with the target sequence 5′-3′ and the ASO sequence from 3′-5′), dividing that number by the total number of nucleotides in the ASO and multiplying by 100. In such a comparison, a nucleobase/nucleotide which does not align (i.e., does not form a base pair) is termed a mismatched nucleobase/nucleotide. Insertions and deletions are not considered in the calculation of percent complementarity of a contiguous nucleotide sequence. It will be understood that in determining complementarity, chemical modifications of the nucleobases are disregarded as long as the functional capacity of the nucleobase to form Watson Crick base-pairing is retained (e.g., 5′-methylcytosine is considered identical to a cytosine for the purpose of calculating % identity). The term “fully complementary,” refers to 100% complementarity.

The term “identity,” as used herein, refers to the proportion of nucleotides (expressed in percent) of a contiguous nucleotide sequence in a nucleic acid molecule (e.g., an ASO of the disclosure) which are identical to a reference sequence (e.g., a sequence motif). The percentage of identity is calculated by counting the number of aligned nucleobases that are identical between two sequences, dividing that number by the total number of nucleotides in the ASO, and multiplying by 100. Insertions and deletions are not considered in the calculation the percentage of identity of a contiguous nucleotide sequence. It will be understood that in determining identity, chemical modifications of the nucleobases are disregarded as long as the functional capacity of the nucleobase to form Watson Crick base-pairing is retained (e.g., 5-methylcytosine is considered identical to a cytosine for the purpose of calculating % identity).

The terms “hybridizing” or “hybridizes,” as used herein, is to be understood as two nucleic acid strands (e.g., an ASO and a target nucleic acid) forming hydrogen bonds between base pairs on opposite strands, thereby forming a duplex between the two strands. The affinity of the binding between two nucleic acid strands is the strength of the hybridization. It is often described in terms of the melting temperature (Tₘ), which is defined as the temperature at which half of the ASOs are duplexed with the TMEM106B target nucleic acid. At physiological conditions, Tₘ is not strictly proportional to the affinity (Mergny and Lacroix, Antisense oligonucleotides 13:515-537 (2003)). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an ASO and the TMEM106B target nucleic acid reflects a strong hybridization between the ASO and target nucleic acid. ΔG° is the energy associated with a reaction where aqueous concentrations of solutes are 1M, the pH is 7, and the temperature is 37° C. The hybridization of ASOs to a target nucleic acid is a spontaneous reaction for which ΔG° is less than zero and is, therefore, thermodynamically favorable. ΔG° can be measured experimentally, for example, by use of the isothermal titration calorimetry (ITC) method as described in Hansen et al., Chem. Comm. 36-38 (1965) and Holdgate et al., Drug Discov Today (2005). A person of ordinary skill in the art will understand that commercial equipment is available for ΔG° measurements. ΔG° can also be estimated numerically by using the nearest neighbor model as described by Santa Lucia, PNAS 95:1460-1465 (1998) using appropriately derived thermodynamic parameters described by Sugimoto et al., Biochemistry 34:11211-11216 (1995) and McTigue et al., Biochemistry 43:5388-5405 (2004). In order to bind its intended nucleic acid target by hybridization, an ASO of the present disclosure hybridizes to a target nucleic acid with estimated ΔG° values below −10 kcal for ASOs that are 10-30 nucleotides in length. The degree or strength of hybridization may be measured by ΔG°. The ASOs may hybridize to a target nucleic acid with estimated ΔG° values below the range of −10 kcal, such as below −15 kcal, such as below −20 kcal, and such as below −25 kcal for ASOs that are 8-30 nucleotides in length. In some cases, the ASO hybridizes to a target nucleic acid with an estimated ΔG° value of −10 to −60 kcal, such as −12 to −40, such as from −15 to −30 kcal or −16 to −27 kcal, such as −18 to −25 kcal.

As used herein, the term “target nucleic acid” is a nucleic acid which encodes mammalian (e.g., human) TMEM106B and may for example be a gene, RNA, mature mRNA, pre-mRNA, or a cDNA sequence. The target may therefore be referred to as a “TMEM106B target nucleic acid” or, simply, “TMEM106B.” The oligonucleotide of the disclosure may for example target an exon of a mammalian TMEM106B RNA or may target an intron in the TMEM106B pre-mRNA (see Table 1). The oligonucleotide may target between the exon-exon boundaries in mRNA and may target the exon-intron boundaries in pre-mRNA.

TABLE 1
Human TMEM106B exonic (e) and intronic (i) target regions

| | | |
|---|---|---|
| ID | start | end |

Exonic regions in the human
TMEM106B pre-mRNA (SEQ ID NO: 1)

| | | |
|---|---|---|
| e1 | 1 | 204 |
| e2 | 2980 | 3173 |
| e3 | 3588 | 3806 |
| e4 | 7237 | 7300 |
| e5 | 13005 | 13164 |
| e6 | 18458 | 18598 |
| e7 | 19168 | 19217 |
| e8 | 19841 | 19894 |
| e9 | 20616 | 32146 |

Intronic regions in the human
TMEM106B pre-mRNA (SEQ ID NO: 1)

| | | |
|---|---|---|
| i1 | 205 | 2979 |
| i2 | 3174 | 3587 |
| i3 | 3807 | 7236 |
| i4 | 7301 | 13004 |
| i5 | 13165 | 18457 |
| i6 | 18599 | 19167 |
| i7 | 19218 | 19840 |
| i8 | 19895 | 20615 |

Suitably, the TMEM106B target nucleic acid encodes a TMEM106B protein, in particular mammalian TMEM106B protein, such as human TMEM106B protein. Tables 2 and 3 provide the mRNA and pre-mRNA sequences for human and monkey TMEM106B.

In some embodiments, the TMEM106B target nucleic acid is selected from the group consisting of SEQ ID NO: 1, 2, 3, and 4, or naturally-occurring variants thereof, including SNP variants. Known single nucleotide polymorphisms (SNPs) of the TMEM106B sequence of SEQ ID NO: 1 are listed in Table 4. If employing the oligonucleotide of the disclosure in research or diagnostics, the TMEM106B target nucleic acid may be a cDNA or a synthetic nucleic acid derived from DNA or RNA.

The ASO of the disclosure is typically capable of inhibiting the expression of the TMEM106B target nucleic acid in a cell, which is expressing the TMEM106B target nucleic acid. The contiguous sequence of nucleobases of the oligonucleotide of the disclosure is typically complementary (fully or partially) to the TMEM106B target nucleic acid, as measured across the length of the ASO, optionally with the exception of one or two mismatches, and optionally excluding nucleotide-based linker regions which may link the ASO to an optional functional group, such as a conjugate, or other non-complementary terminal nucleotides (e.g. region D′ or D″). In some embodiments, the TMEM106B target nucleic acid may be a mature mRNA or a pre-mRNA.

In some embodiments, the TMEM106B target nucleic acid is a RNA which encodes mammalian TMEM106B protein, such as human TMEM106B protein, e.g. the human TMEM106B pre-mRNA sequence, such as that disclosed as SEQ ID NO: 1, or the human mature mRNA, such as that disclosed in SEQ ID NO: 2. Exemplary target nucleic acids are provided in Tables 1-3.

TABLE 2
Genome and assembly information for TMEM106B across species

| | | | | | | NCBI reference |
|---|---|---|---|---|---|---|
| | | | | | | sequence |
| | | | Genomic coordinates | | Assembly/ | accession |
| Species | Chr. | Strand | Start | End | Ensembl Gene ID | number for mRNA |
| Human | 7 | Fwd | 12211222 | 12243367 | GRCh38/ENSG00000106460 | NM_018374 |
| Cynomolgus | 3 | Rv | 108661057 | 108693751 | Macaca_fascicularis_5.0 | XM_005550096 |

Fwd = forward strand. Rv = reverse strand. The genome coordinates provide the pre-mRNA sequence (genomic sequence). The NCBI reference provides the mRNA sequence (cDNA sequence).

TABLE 3
Sequence details for TMEM106B across species

| | RNA | Length | SEQ |
|---|---|---|---|
| Species | type | (nt) | ID NO |
| Human | premRNA | 32146 | 1 |
| Human | mRNA | 6514 | 2 |
| Monkey | premRNA | 32695 | 3 |
| Monkey | mRNA | 2740 | 4 |

The term “target sequence,” as used herein, refers to a sequence of nucleotides present in the TMEM106B target nucleic acid, which includes the nucleobase sequence complementary to the oligonucleotide of the disclosure. In some embodiments, the target sequence consists of a region on the TMEM106B target nucleic acid with a nucleobase sequence that is complementary to the contiguous nucleotide sequence of the oligonucleotide of the disclosure. This region of the TMEM106B target nucleic acid may interchangeably be referred to as the target nucleotide sequence, target sequence, or target region. In some embodiments, the target sequence is longer than the complementary sequence of a 15 single oligonucleotide, and may, for example, represent a preferred region of the TMEM106B target nucleic acid, which may be targeted by several oligonucleotides of the disclosure.

The target sequence to which the ASO is complementary or hybridizes generally includes a contiguous nucleobase sequence of at least 10 nucleotides (e.g., at least 11, 12, 13, 14, 15, 16, 17, 18, 19, 20, 21, 22, 23, 24, 25, 26, 27, 28, 29, or more nucleotides). In some embodiments, the contiguous nucleotide sequence is between 10 to 50 nucleotides, such as 10 to 30 nucleotides, such as 14 to 20, such as 15 to 18 contiguous nucleotides.

The term a “target cell,” as used herein, refers to a cell, which is expressing the TMEM106B target nucleic acid. In some embodiments, the target cell may be in vivo or in vitro. In some embodiments, the target cell is a mammalian cell such as a rodent cell, such as a mouse cell or a rat cell, or a primate cell, such as a monkey cell or a human cell. Exemplary target cells may include neuronal cells, e.g., neuronal cells expressing TMEM106B, such as, astrocytes, oligodendrocytes, microglia, and ependymal cells. The target cell may express TMEM106B mRNA, such as the TMEM106B pre-mRNA or TMEM106B mature mRNA. The polyA tail of TMEM106B mRNA is typically disregarded for ASO targeting. In cases where testing is being performed to assess the knockdown efficacy of ASOs of the disclosure, the term “target cell” may include human SK-N-BE(2) neuroblastoma cells, induced pluripotent stem cell (iPSC)-derived neuron or glial cell, or retinal pigment epithelial cell.

As used herein, the term “naturally-occurring variant” refers to variants of the TMEM106B gene or transcripts thereof, but may differ for example, by virtue of degeneracy of the genetic code, causing a multiplicity of codons encoding the same amino acid, due to alternative splicing of pre-mRNA, or the presence of polymorphisms, such as SNPs and allelic variants. The naturally-occurring variants may have at least 95% (e.g., at least 96%, 97%, 98%, 99%, or more) homology to a mammalian TMEM106B target nucleic acid, such as a target nucleic acid selected form the group consisting of SEQ ID NO: 1-4. The naturally-occurring variants of TMEM106B may also include the SNPs listed in Table 4.

TABLE 4
Examples of known SNPs in the human TMEM106B gene

| | | Ancestral | | | |
|---|---|---|---|---|---|
| | Position | allele (in | | | Minor |
| | in SEQ | reference | Variant | Minor | allele |
| SNP ID | ID NO: 1 | genome) | alleles | allele | frequency |
| rs111783004 | 92 | G | A, G | A | 0.02 |
| rs73299040 | 338 | G | A, G | A | 0.17 |
| rs4721056 | 415 | T | G, T | T | 0.37 |
| rs13223216 | 641 | T | C, T | C | 0.03 |
| rs77;81670 | 713 | C | C, G | C | 0.45 |
| rs73299043 | 748 | C | C, G | G | 0.08 |
| rs73678689 | 822 | T | G, T | G | 0.17 |
| rs1019309 | 824 | T | T, A | T | 0.45 |
| rs77287807 | 862 | A | A, G | G | 0.01 |
| rs1019308 | 879 | G | G, C | G | 0.32 |
| rs1019307 | 943 | G | G, C | G | 0.45 |
| rs1019306 | 979 | C | T, C | C | 0.33 |
| rs57209188 | 1094 | A | A, G | G | 0.02 |
| rs73301009 | 1131 | A | A, G | G | 0.01 |
| rs56761518 | 1273 | — | —, T, TTTT | — | 0.47 |
| rs1468800 | 1296 | A | T, A | T | 0.09 |
| rs6460895 | 1693 | C | C, G | C | 0.41 |
| rs6460896 | 1812 | G | A, G | G | 0.41 |
| rs6460897 | 1894 | T | G, T | T | 0.41 |
| rs73678692 | 1897 | G | A, G | A | 0.13 |
| rs6460898 | 2046 | A | A, G | A | 0.41 |
| rs10644564 | 2056 | — | —, CT | — | 0.41 |
| rs201303186 | 2073 | — | —, A | A | 0.08 |
| rs6460899 | 2203 | A | A, C | A | 0.41 |
| rs6460900 | 2241 | A | A, G | A | 0.41 |
| rs6974465 | 2331 | T | C, T | T | 0.33 |
| rs74964643 | 2372 | G | A, G | A | 0.03 |
| rs12699323 | 2515 | T | C, T | T | 0.41 |
| rs12699324 | 2525 | T | C, T | T | 0.41 |
| rs76235203 | 2612 | C | C, G | G | 0.08 |
| rs10234805 | 2696 | T | C, T | T | 0.41 |
| rs10278257 | 2716 | C | C, T | C | 0.41 |
| rs138861467 | 2851 | C | C, G | G | 0.01 |
| rs2043540 | 3020 | A | C, A | C | 0.28 |
| rs2043539 | 3033 | G | G, A | G | 0.41 |
| rs2043538 | 3046 | T | T, C | C | 0.38 |
| rs73301022 | 3099 | C | A, C | A | 0.02 |
| rs62435714 | 3165 | A | A, G | G | 0.14 |
| rs6959892 | 3421 | C | C, T | T | 0.04 |
| rs17149894 | 4092 | C | C, T | T | 0.30 |
| rs78492048 | 4107 | A | A, G | G | 0.03 |
| rs6460901 | 4587 | C | C, T | C | 0.41 |
| rs6460902 | 4664 | G | A, G | G | 0.39 |
| rs1020006 | 4817 | G | G, A | G | 0.41 |
| rs1020005 | 4821 | C | C, A | C | 0.33 |
| rs1020004 | 4931 | T | T, C | C | 0.30 |
| rs5882345 | 5160 | A | -, A | A | 0.32 |
| rs76689854 | 5275 | T | G, T | G | 0.02 |
| rs11974384 | 5693 | A | A, G | G | 0.38 |
| rs11971133 | 5701 | T | C, T | T | 0.41 |
| rs111886724 | 5802 | G | C, G | C | 0.02 |
| rs577850861 | 5806 | TT | —, TT | — | 0.35 |
| rs574034412 | 5807 | T | —, T | — | 0.49 |
| rs12699325 | 5827 | T | C, T | C | 0.03 |
| rs73677511 | 5863 | T | C, T | C | 0.13 |
| rs73301032 | 5952 | C | C, G | G | 0.08 |
| rs6947327 | 6322 | C | C, G | C | 0.32 |
| rs10464275 | 6381 | A | A, C | C | 0.08 |
| rs73301035 | 6512 | G | A, G | A | 0.14 |
| rs7785189 | 6680 | C | C, T | C | 0.41 |
| rs7785087 | 6702 | A | A, G | A | 0.41 |
| rs6460903 | 6881 | T | C, T | T | 0.33 |
| rs11342660 | 7019 | T | —, T | T | 0.45 |
| rs73301040 | 7079 | A | A, G | G | 0.02 |
| rs6415232 | 7091 | A | A, G | A | 0.33 |
| rs3887296 | 7119 | A | A, G | G | 0.08 |
| rs139417104 | 7220 | — | —, A | A | 0.02 |
| rs116705963 | 7413 | G | A, G | A | 0.01 |
| rs17165735 | 7587 | A | A, G | G | 0.08 |
| rs17165736 | 7675 | G | A, G | A | 0.14 |
| rs35982495 | 7819 | — | —, A | — | 0.40 |
| rs3823612 | 7908 | G | G, C | G | 0.40 |
| rs202184519 | 8740 | — | —, A | A | 0.01 |
| rs17165737 | 8788 | C | A, C | A | 0.13 |
| rs78663549 | 9001 | G | G, T | T | 0.09 |
| rs28459566 | 9243 | T | C, T | C | 0.11 |
| rs12666249 | 9699 | C | C, T | T | 0.38 |
| rs12666279 | 9705 | G | A, G | A | 0.14 |
| rs12699326 | 9719 | A | A, G | G | 0.03 |
| rs139345875 | 9974 | A | A, T | T | 0.01 |
| rs147697289 | 10156 | G | G, T | T | 0.01 |
| rs73301046 | 10400 | G | A, G | A | 0.05 |
| rs80041000 | 10520 | A | A, G | G | 0.08 |
| rs10950391 | 10982 | A | A, G | A | 0.40 |
| rs11509880 | 11064 | G | A, G | G | 0.42 |
| rs141482004 | 11096 | A | A, T | T | 0.01 |
| rs76188878 | 11318 | G | A, G | A | 0.02 |
| rs3800847 | 11395 | G | G, A | G | 0.40 |
| rs1435527 | 11724 | C | T, C | C | 0.41 |
| rs6978684 | 11740 | C | C, G | G | 0.14 |
| rs13311466 | 11755 | T | C, T | C | 0.03 |
| rs1435526 | 11807 | A | G, A | A | 0.33 |
| rs1435525 | 11870 | T | T, C | T | 0.41 |
| rs1435524 | 11954 | G | G, A | G | 0.41 |
| rs11368032 | 12122 | T | —, T | T | 0.41 |
| rs55635711 | 12156 | A | —, A | — | 0.12 |
| rs73677523 | 12199 | A | A, G | G | 0.09 |
| rs73301054 | 12213 | C | A, C | A | 0.04 |
| rs115923636 | 12244 | A | A, G | G | 0.01 |
| rs5882346 | 12337 | A | —, A | A | 0.41 |
| rs1435543 | 12387 | C | T, C | C | 0.32 |
| rs17165744 | 12442 | A | A, G | G | 0.13 |
| rs76130803 | 12457 | T | C, T | C | 0.02 |
| rs11981005 | 12523 | A | A, G | A | 0.32 |
| rs11974335 | 12531 | G | G, T | G | 0.40 |
| rs11974304 | 12590 | C | C, T | C | 0.40 |
| rs10950392 | 12691 | T | C, T | T | 0.40 |
| rs10950393 | 12699 | T | C, T | T | 0.40 |
| rs10950394 | 12740 | C | C, T | C | 0.40 |
| rs144830416 | 12745 | G | C, G | C | 0.08 |
| rs113404588 | 12766 | G | A, G | A | 0.05 |
| rs141956872 | 12776 | G | C, G | C | 0.01 |
| rs116565082 | 12794 | C | C, T | T | 0.03 |
| rs113047067 | 12829 | C | C, G | G | 0.03 |
| rs17165746 | 12878 | T | A, T | A | 0.14 |
| rs11509137 | 12952 | T | C, T | T | 0.40 |
| rs11509153 | 12953 | G | A, G | G | 0.41 |
| rs147889591 | 13124 | G | A, G | A | 0.01 |
| rs3800845 | 13304 | G | G, C | G | 0.33 |
| rs3800844 | 13373 | G | G, C | G | 0.33 |
| rs3839693 | 13415 | — | —, A | — | 0.40 |
| rs3800843 | 13450 | G | G, A | G | 0.40 |
| rs75163637 | 13487 | A | A, G | G | 0.01 |
| rs77799203 | 13585 | C | C, G | G | 0.06 |
| rs10950395 | 13620 | T | C, T | T | 0.40 |
| rs149105187 | 13795 | C | C, T | T | 0.02 |
| rs10950396 | 13819 | T | C, T | T | 0.40 |
| rs11983898 | 13862 | G | A, G | A | 0.27 |
| rs10950397 | 13890 | C | C, T | C | 0.41 |
| rs137900300 | 13937 | G | A, G | A | 0.03 |
| rs10950398 | 14024 | G | A, G | G | 0.39 |
| rs76854159 | 14212 | A | A, G | A | 0.39 |
| rs138328979 | 14275 | G | C, G | C | 0.01 |
| rs79222644 | 14302 | A | A, T | T | 0.06 |
| rs150465020 | 14345 | T | C, T | T | 0.40 |
| rs147309966 | 14427 | G | G, T | T | 0.27 |
| rs140918518 | 14440 | A | A, G | G | 0.01 |
| rs143141037 | 14462 | C | C, T | T | 0.04 |
| rs13246340 | 14476 | G | A, G | A | 0.03 |
| rs142820543 | 14492 | G | A, G | A | 0.04 |
| rs75738247 | 14583 | G | G, T | T | 0.27 |
| rs139628525 | 14595 | C | C, G | G | 0.01 |
| rs143477885 | 14618 | A | A, G | G | 0.14 |
| rs201845319 | 14754 | T | —, T | — | 0.02 |
| rs112430481 | 14891 | C | C, G | G | 0.08 |
| rs6966602 | 14892 | C | C, G | G | 0.30 |
| rs145413472 | 14911 | C | C, T | T | 0.11 |
| rs11438918 | 14921 | — | —, T | — | 0.33 |
| rs6948844 | 15001 | T | C, T | T | 0.40 |
| rs6967026 | 15058 | G | G, T | T | 0.27 |
| rs73284389 | 15080 | C | C, G | G | 0.14 |
| rs6966757 | 15105 | A | A, T | A | 0.40 |
| rs6966915 | 15141 | C | C, T | C | 0.41 |
| rs77145882 | 15152 | G | A, G | A | 0.09 |
| rs115474060 | 15154 | C | C, G | G | 0.02 |
| rs143032237 | 15190 | G | C, G | C | 0.01 |
| rs17165750 | 15272 | T | C, T | C | 0.14 |
| rs117574031 | 15285 | C | C, G | G | 0.01 |
| rs571665718 | 15455 | T | A, T | A | 0.01 |
| rs539782765 | 15456 | C | C, T | T | 0.01 |
| rs3839692 | 15474 | — | —, A | A | 0.30 |
| rs35642626 | 15705 | A | —, A | A | 0.40 |
| rs75275529 | 15780 | A | A, G | G | 0.08 |
| rs7804433 | 15859 | G | A, G | G | 0.39 |
| rs61225336 | 15864 | T | C, T | C | 0.19 |
| rs7804234 | 15967 | A | A, G | A | 0.40 |
| rs7804736 | 16020 | G | G, T | G | 0.41 |
| rs78536513 | 16336 | A | A, C | C | 0.02 |
| rs4721057 | 16374 | A | A, G | A | 0.40 |
| rs4721058 | 16409 | C | C, T | C | 0.40 |
| rs73286304 | 16568 | C | A, C | A | 0.03 |
| rs4721059 | 16648 | G | C, G | G | 0.40 |
| rs4721060 | 16705 | G | A, G | G | 0.40 |
| rs4721061 | 16712 | G | C, G | G | 0.40 |
| rs7792410 | 16767 | T | C, T | T | 0.40 |
| rs536653563 | 16768 | C | C, T | T | 0.04 |
| rs35390376 | 16806 | T | C, T | C | 0.03 |
| rs7809700 | 16887 | G | A, G | G | 0.40 |
| rs115298566 | 16902 | G | G, T | T | 0.01 |
| rs13229988 | 16990 | A | A, G | A | 0.40 |
| rs149578700 | 17020 | C | C, T | T | 0.02 |
| rs201723928 | 17022 | T | —, T | I | 0.01 |
| rs34046032 | 17236 | TTAA | —, TTAA | TTAA | 0.40 |
| rs28549831 | 17270 | C | C, T | C | 0.40 |
| rs13230513 | 17396 | C | A, C | C | 0.40 |
| rs12667950 | 17621 | G | G, T | G | 0.41 |
| rs5011431 | 17704 | G | A, G | G | 0.40 |
| rs5011432 | 17821 | A | A, C | A | 0.48 |
| rs5011433 | 17848 | T | A, T | T | 0.32 |
| rs5011434 | 17870 | C | C, T | C | 0.40 |
| rs5011435 | 17879 | T | C, T | C | 0.13 |
| rs5011436 | 17911 | A | A, C | A | 0.40 |
| rs5011437 | 17945 | A | A, T | A | 0.40 |
| rs5011438 | 17951 | C | C, T | C | 0.40 |
| rs5011439 | 17964 | G | C, G | G | 0.40 |
| rs13233991 | 18067 | C | C, T | — | 0.03 |
| rs60699002 | 18199 | C | A, C | A | 0.09 |
| rs13234238 | 18366 | A | A, C | C | 0.03 |
| rs3839691 | 18414 | — | —, TTGT | TTGT | 0.49 |
| rs3173615 | 18570 | C | C, G | G | 0.50 |
| rs13237715 | 18728 | G | C, G | G | 0.41 |
| rs13237518 | 18746 | C | A, C | C | 0.39 |
| rs59265139 | 18794 | G | A, G | A | 0.04 |
| rs143516511 | 18796 | G | A, G | A | 0.01 |
| rs35562417 | 18833 | — | —, A, AA | — | 0.41 |
| rs114069088 | 18885 | T | A, T | A | 0.01 |
| rs12699332 | 18915 | G | G, T | G | 0.41 |
| rs12699333 | 18957 | C | C, T | C | 0.40 |
| rs12668625 | 18970 | G | A, G | G | 0.40 |
| rs3815535 | 19672 | G | G, A | G | 0.40 |
| rs3214372 | 19719 | — | —, T | — | 0.41 |
| rs3217132 | 19737 | TTA | —, ATT | — | 0.14 |
| rs2302635 | 19778 | G | G, A | G | 0.41 |
| rs2302634 | 19923 | T | T, A | A | 0.50 |
| rs73677558 | 19944 | T | C, T | C | 0.10 |
| rs2302633 | 19968 | C | T, C | C | 0.41 |
| rs73677559 | 19972 | A | A, C | C | 0.08 |
| rs16877361 | 20053 | C | C, T | T | 0.17 |
| rs2302632 | 20120 | A | G, A | G | 0.36 |
| rs7808568 | 20405 | T | C, T | T | 0.39 |
| rs12699334 | 20406 | G | A, G | A | 0.03 |
| rs3800842 | 20605 | A | G, C, A | A | 0.40 |
| rs1042946 | 20819 | G | G, T | T | 0.03 |
| rs12669919 | 21150 | G | A, G | G | 0.41 |
| rs16877363 | 21179 | A | A, G | G | 0.14 |
| rs1042949 | 21269 | C | C, G | C | 0.41 |
| rs3800841 | 21721 | A | T, A | A | 0.40 |
| rs17165789 | 21903 | A | A, C | C | 0.03 |
| rs34252347 | 22017 | — | —, TG | TG | 0.44 |
| rs14978 | 22305 | A | G, A | G | 0.43 |
| rs1054168 | 22566 | C | C, T | C | 0.40 |
| rs1054169 | 22649 | G | A, G | G | 0.40 |
| rs79340241 | 22780 | — | —, A | A | 0.08 |
| rs12539421 | 22924 | T | G, T | G | 0.08 |
| rs13309255 | 23361 | G | G, T | T | 0.03 |
| rs10488193 | 23373 | A | G, A | G | 0.09 |
| rs35337387 | 23567 | — | —, T | — | 0.40 |
| rs17165790 | 24003 | A | A, G | G | 0.01 |
| rs71529336 | 24086 | A | A, G | G | 0.03 |
| rs1468804 | 24661 | T | T, C | T | 0.39 |
| rs5882347 | 24778 | — | —, TTTTC | — | 0.40 |
| rs1468803 | 24828 | A | C, A | A | 0.39 |
| rs1060700 | 24971 | A | G, A | A | 0.39 |
| rs73288318 | 25015 | G | A, G | A | 0.03 |
| rs1468802 | 25164 | G | G, A | G | 0.39 |
| rs2160268 | 25178 | A | T, A | T | 0.13 |
| rs2160267 | 25187 | T | T, A | A | 0.33 |
| rs1468801 | 25198 | G | G, C | G | 0.39 |
| rs1047601 | 25674 | C | C, T | T | 0.08 |
| rs929637 | 25675 | G | T, G | T | 0.33 |
| rs57824581 | 25834 | — | —, A | A | 0.05 |
| rs112281135 | 25903 | — | —, ATG | — | 0.01 |
| rs117623176 | 25985 | C | C, T | T | 0.01 |
| rs2356065 | 26038 | T | C, T | T | 0.39 |
| rs140537895 | 26149 | TACTT | —, TACTT | — | 0.09 |
| rs12699335 | 26173 | T | C, T | C | 0.36 |
| rs10488192 | 26234 | G | G, A | A | 0.14 |
| rs199711002 | 26589 | T | C, T | C | 0.13 |
| rs13234969 | 26591 | T | C, T | T | 0.43 |
| rs13234970 | 26595 | T | C, T | T | 0.43 |
| rs7797705 | 26926 | G | A, G | G | 0.40 |
| rs6460904 | 27302 | G | A, G | A | 0.17 |
| rs34391411 | 27560 | CAA | —, CAA | | 0.03 |
| rs6969722 | 27590 | G | A, G | A | 0.35 |
| rs4330594 | 27768 | G | A, G | A | 0.07 |
| rs62448695 | 27946 | T | C, T | C | 0.05 |
| rs73288387 | 27958 | T | C, T | C | 0.05 |
| rs10257722 | 28065 | G | A, G | A | 0.01 |
| rs548644997 | 28119 | T | G, T | G | 0.21 |
| rs7802952 | 28280 | A | A, G | G | 0.13 |
| rs2098363 | 28351 | G | G, A | G | 0.33 |
| rs57685335 | 28672 | G | A, G | A | 0.04 |
| rs1548885 | 28712 | G | G, C | C | 0.35 |
| rs75956572 | 28891 | A | A, G | G | 0.03 |
| rs1548884 | 28914 | A | C, A | A | 0.39 |
| rs1548883 | 28946 | G | G, A | A | 0.13 |
| rs2356066 | 28986 | G | A, G | A | 0.35 |
| rs73288393 | 29090 | T | G, T | G | 0.04 |
| rs77273680 | 29280 | A | A, G | G | 0.08 |
| rs73677570 | 29482 | T | C, T | C | 0.07 |
| rs149200435 | 29591 | AGA | —, AGA | — | 0.08 |
| rs79100376 | 29598 | C | C, G | G | 0.09 |
| rs75911624 | 29661 | A | A, G | G | 0.02 |
| rs10281425 | 29883 | T | A, T | T | 0.39 |
| rs150586332 | 29900 | C | C, G | G | 0.01 |
| rs10236369 | 29991 | A | A, G | G | 0.37 |
| rs116132326 | 30039 | T | A, T | A | 0.02 |
| rs570235340 | 30253 | — | —, | TCCCACTTATG | 0.04 |
| | | | TCCCACTTATGAG | AGTGGCAACT | |
| | | | TGGCAACT (SEQ | (SEQ ID | |
| | | | ID NO: 51) | NO: 51) | |
| rs139600599 | 30445 | G | G, T | T | 0.07 |
| rs115077070 | 30833 | T | G, T | G | 0.02 |
| rs139393760 | 30857 | A | —, A | A | 0.39 |
| rs6948681 | 31146 | G | A, G | A | 0.10 |
| rs146083730 | 31293 | — | —, T | T | 0.01 |
| rs7805419 | 31604 | T | C, T | T | 0.46 |
| rs149298119 | 31821 | A | A, T | T | 0.01 |
| rs59410610 | 32000 | A | A, G | G | 0.07 |

The term “modulation of expression,” as used herein, is to be understood as an overall term for an ASO's ability to alter the amount of TMEM106B when compared to the amount of TMEM106B before administration of the ASO. Alternatively, modulation of expression may be determined by reference to a control experiment. It is generally understood that the control is an individual or target cell treated with a saline composition or an individual or target cell treated with a non-targeting ASO (scramble or mock control). One type of modulation is the ability of an ASO to inhibit, downregulate, reduce, suppress, remove, stop, block, prevent, lessen, lower, or terminate expression of TMEM106B, e.g., by degradation of mRNA or repression of transcription. Another type of modulation is an ASO's ability to restore, increase, or enhance expression of TMEM106B, e.g., by repair of splice sites or prevention of splicing or removal or blockage of inhibitory mechanisms such as microRNA repression. Within the context of the present disclosure, the ASOs described herein act to reduce or suppress TMEM106B expression.

As used herein, a “high-affinity modified nucleoside” is a modified nucleotide which, when incorporated into the ASO, enhances the affinity of the ASO for its complementary target, e.g., as measured by the Tₘ. A high-affinity modified nucleoside of the present disclosure may increase the Tₘ by about +0.5 to +12° C., +1.5 to +10° C., or +3 to +8° C. per modified nucleoside. Numerous high-affinity modified nucleosides are known in the art and include, for example, 2′-substituted nucleosides as well as locked nucleic acids (LNA) (see Freier et al., Nucl. Acid Res. 25:4429-43 (1997) and Uhlmann, Curr. Opinion in Drug Development 3:293-213 (2000).

The oligomer of the disclosure may include one or more nucleosides which have a modified sugar moiety. Numerous nucleosides with modification of the ribose sugar moiety have been made, primarily with the aim of improving certain properties of ASOs, such as affinity, nuclease resistance, or pharmacokinetics. Such modifications include those where the ribose ring structure is modified, e.g., by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar-modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example, in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may be introduced at the 2′, 3′, 4′, or 5′ positions of the sugar moiety.

As used herein, a “2′ sugar-modified nucleoside” is a nucleoside which has a substituent other than H or —OH at the 2′ position (e.g., a 2′-substituted nucleoside) or includes a 2′ linked biradical capable of forming a bridge between the 2′ carbon and a second carbon in the ribose ring, such as LNA (2′-4′ biradical bridged) nucleosides. Numerous 2′-substituted nucleosides have been found to have beneficial properties when incorporated into ASOs. For example, the 2′-modified sugar may enhance binding affinity or increase nuclease resistance of the ASO. Examples of 2′-substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. For further examples (see e.g. Freier et al., Nucl. Acid Res. 25:4429-4443 (1997) and Uhlmann Curr. Opinion in Drug Development 3:293-313 (2000), and Deleavey et al., Chemistry and Biology 19: 937 (2012). Shown below are illustrations of some 2′-modified nucleosides.

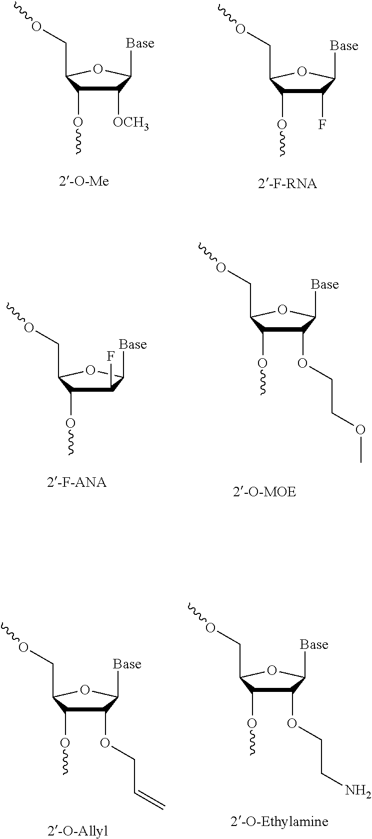

A “LNA nucleoside” is a 2′-modified nucleoside which includes a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the ribose is associated with an enhanced affinity of hybridization (duplex stabilization) when the LNA is incorporated into an ASO. This can be routinely determined by measuring the melting temperature of the antisense oligonucleotide/target duplex.

Non-limiting examples of LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorg. Med. Chem. Lett. 12:73-6 (2002), Seth et al., J. Org. Chem. 75:1569-81 (2010), Mitsuoka et al., Nucleic Acid Res. 37:1225-38 (2009), and Wan et al., J. Med. Chem. 59:9645-67 (2016). Other exemplary LNA nucleosides are disclosed in Scheme 1, below.

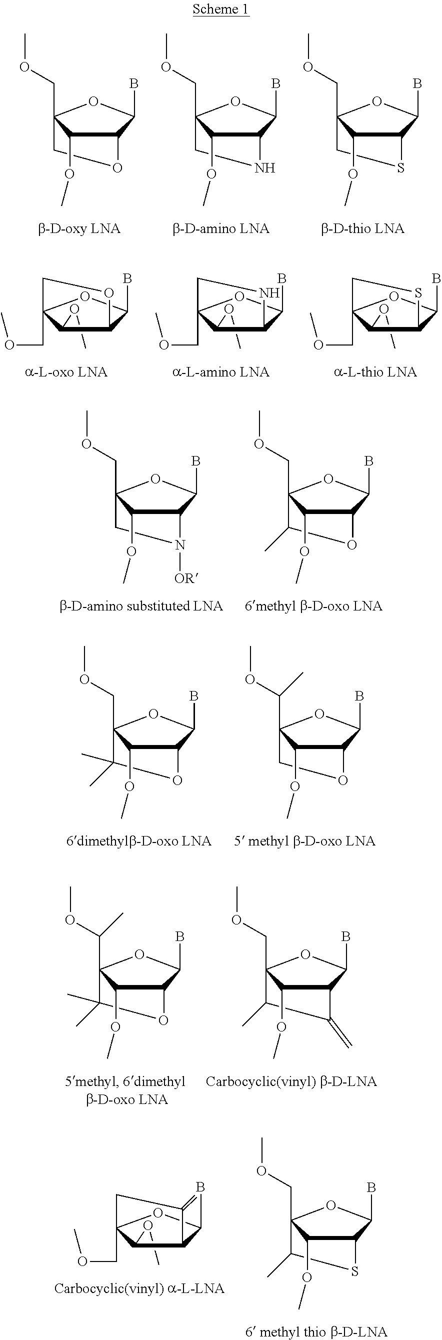

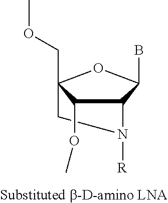

The LNA nucleosides may be beta-D-oxy-LNA, 6′-methyl-beta-D-oxy LNA such as(S)-6′-methyl-beta-D-oxy-LNA (ScET), and ENA. A particularly advantageous LNA is a beta-D-oxy-LNA.

Exemplary Nucleosides, with HELM Annotation:

### DNA Nucleosides

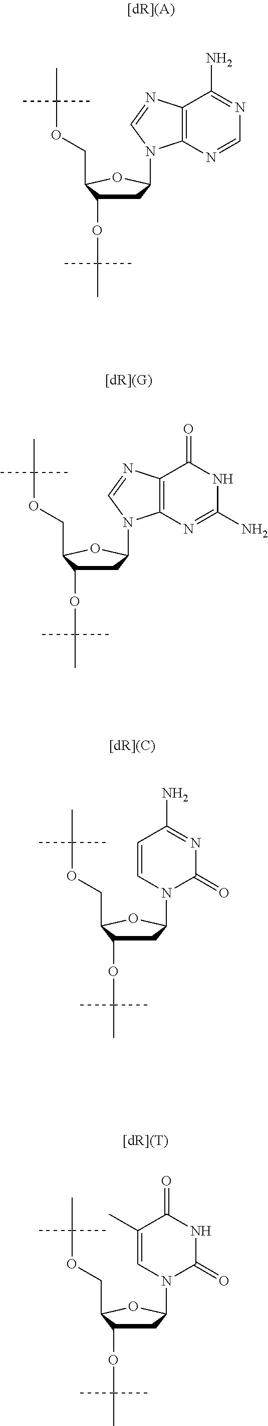

### Beta-D-Oxy-LNA Nucleosides

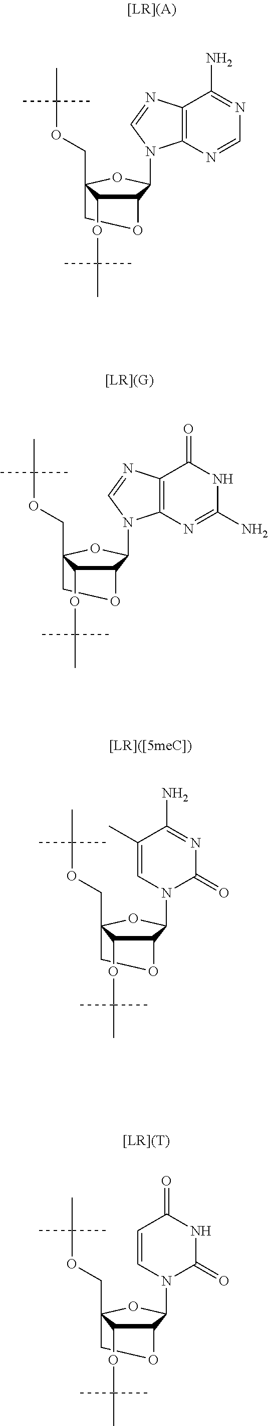

### 2′-O-Methyl Nucleosides

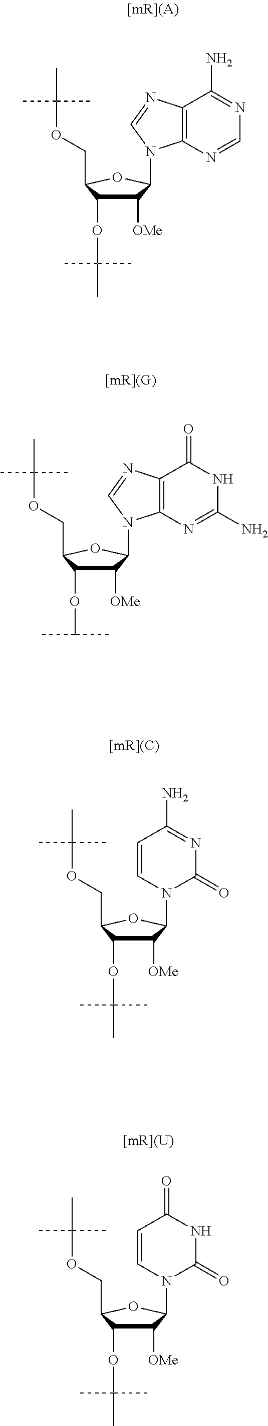

Inverted Nucleosides (when Integrated in Oligonucleotide with Phosphodiester Bonds)

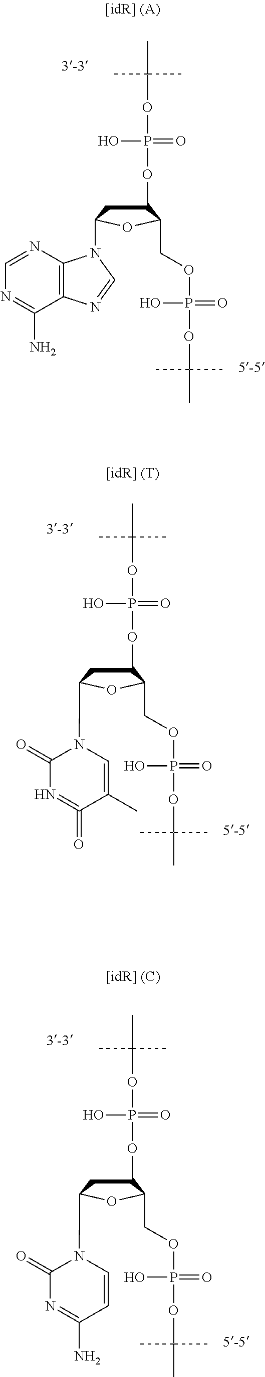

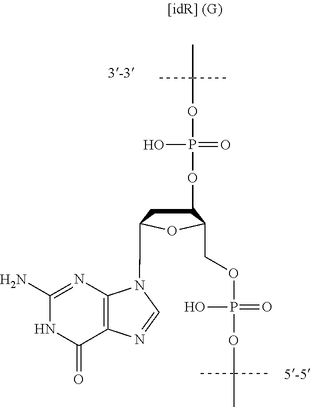

### DNA 7-Deaza-8-Aza-Guanine Nucleosides

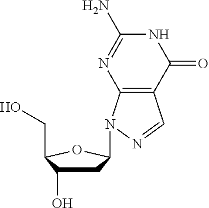

Exemplary Phosphorothioate Internucleoside Linkage with HELM Annotation [sP]

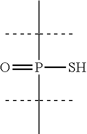

Exemplary Phosphodiester Internucleoside Linkage with HELM Annotation [P]

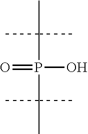

The dashed lines represent the covalent bond between each nucleoside and the 5′ or 3′ phosphorothioate internucleoside linkages. At the 5′ terminal nucleoside, the 5′ dotted lines represent a bond to a hydrogen atom (forming a 5′ terminal-OH group). At the 3′ terminal nucleoside, the 3′ dotted lines represent a bond to a hydrogen atom (forming a 3′ terminal-OH group).

Nuclease mediated degradation refers to an ASO capable of mediating degradation of a complementary nucleotide sequence when forming a duplex with such a sequence. In some embodiments, the ASO may function via nuclease mediated degradation of the TMEM106B target nucleic acid, where the ASOs of the disclosure are capable of recruiting a nuclease, particularly and endonuclease, preferably endoribonuclease (RNase), such as RNase H. Examples of ASO designs which operate via nuclease mediated mechanisms are ASOs which typically include a region of at least 5 or 6 consecutive DNA nucleosides and are flanked on one side or both sides by affinity enhancing nucleosides, for example gapmers, headmers and tailmers.

The RNase H activity of an ASO refers to its ability to recruit RNase H when in a duplex with a complementary RNA molecule. WO01/23613 provides in vitro methods for determining RNase H activity, which may be used to determine the ability to recruit RNase H. Typically an ASO is deemed capable of recruiting RNase H if it, when provided with a complementary target nucleic acid sequence, has an initial rate, as measured in pmol/l/min, of at least 5%, such as at least 10% or more than 20% of the of the initial rate determined when using a ASO having the same base sequence as the modified ASO being tested, but containing only DNA monomers with phosphorothioate linkages between all monomers in the ASO, and using the methodology provided by Example 91-95 of WO01/23613 (hereby incorporated by reference). For use in determining RNase H activity, recombinant human RNase H1 is available from Lubio Science GmbH, Lucerne, Switzerland.

An ASO of the disclosure, or a contiguous nucleotide sequence thereof, may be a gapmer, also termed gapmer ASO or gapmer designs. The antisense gapmers are commonly used to inhibit a target nucleic acid via RNase H mediated degradation. A gapmer ASO includes at least three distinct structural regions a 5′-flank, a gap and a 3′-flank, F-G-F′ in the ‘5->3’ orientation. The “gap” region (G) includes a stretch of contiguous DNA nucleotides which enable the ASO to recruit RNase H. The gap region is flanked by a 5′ flanking region (F) including one or more sugar-modified nucleosides, advantageously high-affinity sugar-modified nucleosides, and by a 3′ flanking region (F′) including one or more sugar-modified nucleosides, advantageously high-affinity sugar-modified nucleosides. The one or more sugar-modified nucleosides in region F and F′ enhance the affinity of the ASO for the TMEM106B target nucleic acid (i.e., are affinity enhancing sugar-modified nucleosides). In some embodiments, the one or more sugar-modified nucleosides in region F and F′ are 2′ sugar-modified nucleosides, such as high-affinity 2′ sugar modifications, such as independently selected from LNA and 2′-MOE.

In a gapmer design, the 5′ and 3′ most nucleosides of the gap region are DNA nucleosides and are positioned adjacent to a sugar-modified nucleoside of the 5′ (F) or 3′ (F′) regions respectively. The flanks may further be defined by having at least one sugar-modified nucleoside at the end most distant from the gap region, i.e., at the 5′ end of the 5′ flank and at the 3′ end of the 3′ flank.

Regions F-G-F′ form a contiguous nucleotide sequence. ASOs of the disclosure, or the contiguous nucleotide sequences thereof, may include a gapmer region of formula F-G-F′.

The overall length of the gapmer design F-G-F′ may be, for example 12 to 32 nucleosides, such as 13 to 24, such as 14 to 22 nucleosides, such as from 14 to 17, such as 16 to 18 nucleosides, such as 16 to 20 nucleotides.

By way of example, the gapmer ASO of the present disclosure can be represented by the following formulae: F₁₋₈-G₆₋₁₆-F′₂₋₈, such as, e.g., F₂₋₈-G₆₋₁₄-F′₂₋₈, such as, e.g., F₃₋₈-G₆₋₁₄-F′₂₋₈; with the proviso that the overall length of the gapmer regions F-G-F′ is at least 10, such as at least 12, such as at least 14 nucleotides in length.

In an aspect of the disclosure the ASO or contiguous nucleotide sequence thereof consists of or includes a gapmer of formula 5′-F-G-F′-3′, where region F and F′ independently include or consist of 1-8 nucleosides, of which 2-4 are 2′ sugar-modified and defines the 5′ and 3′ end of the F and F′ region, and G is a region between 6 and 16 nucleosides which are capable of recruiting RNase H. Regions F, G, and F′ are further defined below and can be incorporated into the F-G-F′ formula.

Region G (gap region) of the gapmer is a region of nucleosides which enables the ASO to recruit RNase H, such as human RNase H1, typically DNA nucleosides. RNase H is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5′-methylcytosine (^{me}C or with an E instead of a C). Methylation of cytosine DNA in the gap is advantageous if CG dinucleotides are present in the gap to improve therapeutic safety, the modification does not have significant impact on efficacy of the ASOs. 5′ substituted DNA nucleosides, such as 5′ methyl DNA nucleoside have been reported for use in DNA gap regions (EP 2 742 136).

In some embodiments, the gap region G may consist of 6, 7, 8, 9, 10, 11, 12, 13, 14, 15, or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNase H recruitment when they are used within the gap region. Modified nucleosides which have been reported as being capable of recruiting RNase H when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e., modifications which allow for RNase H recruitment). In some embodiments the DNA Gap region (G) described herein may optionally contain 1 to 3 sugar-modified nucleosides which adopt a 2′ endo (DNA-like) structure when introduced into the gap region.

Alternatively, there are numerous reports of the insertion of a modified nucleoside which confers a 3′ endo conformation into the gap region of gapmers, whilst retaining some RNase H activity. Such gapmers with a gap region including one or more 3′endo modified nucleosides are referred to as “gap-breaker” or “gap-disrupted” gapmers, see for example WO2013/022984. Gap-breaker ASOs retain sufficient region of DNA nucleosides within the gap region to allow for RNase H recruitment. The ability of gap-breaker ASO design to recruit RNase H is typically sequence or even compound specific-see Rukov et al. 2015 Nucl. Acids Res. Vol. 43 pp. 8476-8487, which discloses “gap-breaker” ASOs which recruit RNase H which in some instances provide a more specific cleavage of the target RNA. Modified nucleosides used within the gap region of gap-breaker ASOs may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which include a disrupted gap typically retain a region of at least 3 or 4 contiguous DNA nucleosides at either the 5′ end or 3′ end of the gap region.

Exemplary designs for gap-breaker ASOs include: F₁₋₈-[D₃₋₄-E₁-D₃₋₄]-F′₁₋₈; F₁₋₈-[D₁₋₄-E₁-D₃₋₄]-F′₁₋₈; and F₁₋₈-[D₃₋₄-E₁-D₁₋₄]-F′₁₋₈; wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length. In some embodiments, region G of a gap disrupted gapmer includes at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNase H recruitment.

Region F is positioned immediately adjacent to the 5′ DNA nucleoside of region G. The 3′ most nucleoside of region F is a sugar-modified nucleoside, such as a high-affinity sugar-modified nucleoside, for example a 2′-substituted nucleoside, such as a MOE nucleoside, or an LNA nucleoside. Region F′ is positioned immediately adjacent to the 3′ DNA nucleoside of region G. The 5′ most nucleoside of region F′ is a sugar-modified nucleoside, such as a high-affinity sugar-modified nucleoside, for example a 2′-substituted nucleoside, such as a MOE nucleoside, or an LNA nucleoside. Region F is 1-8 contiguous nucleotides in length, such as 2-6, such as 3-4 contiguous nucleotides in length. Advantageously the 5′ most nucleoside of region F is a sugar-modified nucleoside. In some embodiments the two 5′ most nucleoside of region F are sugar-modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′-substituted nucleoside nucleosides, such as two 3′ MOE nucleosides. In some embodiments the 5′ most nucleoside of region F is a 2′-substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar-modified nucleoside. In some embodiments the two 3′ most nucleoside of region F′ are sugar-modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′-substituted nucleoside nucleosides, such as two 3′ MOE nucleosides. In some embodiments the 3′ most nucleoside of region F′ is a 2′-substituted nucleoside, such as a MOE nucleoside. It should be noted that when the length of region F or F′ is one, it is advantageously an LNA nucleoside.

In some embodiments, regions F and F′ independently consist of or include a contiguous sequence of sugar-modified nucleosides. In some embodiments, the sugar-modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units, and 2′-fluoro-ANA units. In some embodiments, regions F and F′ independently include both LNA and a 2′-substituted modified nucleosides (mixed-wing design).

In some embodiments, regions F and F′ consist of only one type of sugar-modified nucleosides, such as only MOE or only beta-D-oxy LNA or only ScET. Such designs are also termed uniform flanks or uniform gapmer design.

In some embodiments, all the nucleosides of region F or F′ are LNA nucleosides, such as independently selected from beta-D-oxy LNA, ENA or ScET nucleosides. In some embodiments, region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′ are 2′-substituted nucleosides, such as OMe or MOE nucleosides. In some embodiments, region F consists of 1, 2, 3, 4, 5, 6, 7, or 8 contiguous OMe or MOE nucleosides. In some embodiments only one of the flanking regions can consist of 2′-substituted nucleosides, such as OMe or MOE nucleosides. In some embodiments it is the 5′ (F) flanking region that consists of 2′-substituted nucleosides, such as OMe or MOE nucleosides whereas the 3′ (F′) flanking region includes at least one LNA nucleoside, such as beta-D-oxy LNA nucleosides or cET nucleosides. In some embodiments it is the 3′ (F′) flanking region that consists of 2′-substituted nucleosides, such as OMe or MOE nucleosides whereas the 5′ (F) flanking region includes at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′ may optionally include DNA nucleosides (an alternating flank, see definition of these for more details). In some embodiments, all the modified nucleosides of region F and F′ are beta-D-oxy LNA nucleosides, wherein region F or F′ may optionally include DNA nucleosides (an alternating flank, see definition of these for more details). In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

A “LNA gapmer” is a gapmer wherein either one or both of region F and F′ includes or consists of LNA nucleosides. A beta-D-oxy gapmer is a gapmer wherein either one or both of region F and F′ includes or consists of beta-D-oxy LNA nucleosides. In some embodiments the LNA gapmer is of formula: [LNA]₁₋₅-[region G]-[LNA]₁₋₅, wherein region G is as defined in the Gapmer region G definition. In one embodiment the LNA gapmer is of the formula [LNA]₄-[region G]₁₀₋₁₂-[LNA]₄

A “MOE gapmer” is a gapmer wherein regions F and F′ consist of MOE nucleosides. In some embodiments the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, wherein region G is as defined in the gapmer definition. MOE gapmers with a 5-10-5 design (MOE-DNA-MOE) have been widely used in the art.

A “mixed wing gapmer” is an LNA gapmer wherein one or both of region F and F′ include a 2′-substituted nucleoside, such as a 2′-substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as MOE nucleosides. In some embodiments wherein at least one of region F and F′, or both region F and F′ include at least one LNA nucleoside, the remaining nucleosides of region F and F′ are independently selected from the group consisting of MOE and LNA. In some embodiments wherein at least one of region F and F′, or both region F and F′ include at least two LNA nucleosides, the remaining nucleosides of region F and F′ are independently selected from the group consisting of MOE and LNA. In some mixed wing embodiments, one or both of region F and F′ may further include one or more DNA nucleosides. Mixed wing gapmer designs are disclosed in WO2008/049085 and WO2012/109395, both of which are hereby incorporated by reference.

Flanking regions may include both LNA and DNA nucleoside and are referred to as “alternating flanks” as they include an alternating motif of LNA-DNA-LNA nucleosides. Gapmers including such alternating flanks are referred to as “alternating flank gapmers”. “Alternative flank gapmers” are LNA gapmer ASOs where at least one of the flanks (F or F′) includes DNA in addition to the LNA nucleoside(s). In some embodiments at least one of region F or F′, or both region F and F′, include both LNA nucleosides and DNA nucleosides. In such embodiments, the flanking region F or F′, or both F and F′ include at least three nucleosides, wherein the 5′ and 3′ most nucleosides of the F or F′ region are LNA nucleosides. Alternating flank LNA gapmers are disclosed in WO2016/127002. An alternating flank region may include up to 3 contiguous DNA nucleosides, such as 1 to 2 or 1 or 2 or 3 contiguous DNA nucleosides. The alternating flak can be annotated as a series of integers, representing a number of LNA nucleosides (L) followed by a number of DNA nucleosides (D), for example: [L]₁₋₃-[D]₁₋₄-[L]₁₋₃ or [L]₁₋₂-[D]₁₋₂-[L]₁₋₂-[D]₁₋₂-[L]₁₋₂.

In ASO designs these will often be represented as numbers such that 2-2-1 represents 5′ [L]₂-[D]₂-[L] 3′, and 1-1-1-1-1 represents 5′ [L]-[D]-[L]-[D]-[L] 3′. The length of the flank (region F and F′) in ASOs with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer ASO is alternating while the other is constituted of LNA nucleotides. It may be advantageous to have at least two LNA nucleosides at the 3′ end of the 3′ flank (F′), to confer additional exonuclease resistance. In one embodiment the flanks in the alternating flank gapmer have an overall length from 5- to 8 nucleosides of which 3 to 5 are LNA nucleosides. Some examples of ASOs with alternating flanks are: [L]₁₋₅-[D]₁₋₄-[L]₁₋₃-[G]₅₋₁₆-[L]₂₋₆; [L]₁₋₂-[D]₂₋₃-[L]₃₋₄-[G]₅₋₇-[L]₁₋₂-[D]₂₋₃-[L]₂₋₃; [L]₁₋₂-[D]₁₋₂-[L]₁₋₂-[D]₁₋₂-[L]₁₋₂-[G]₅₋₁₆-[L]₁₋₂-[D]₁₋₃-[L]₂₋₄; [L]₁₋₅-[G]₅₋₁₆-[L]-[D]-[L]-[D]-[L]₂; and [L]₄-[G]₆₋₁₀-[L]-[D]₃-[L]₂; with the proviso that the overall length of the gapmer is at least 12, such as at least 14, nucleotides in length.

The ASOs of the disclosure may in some embodiments include or consist of the contiguous nucleotide sequence of the ASO which is complementary to the TMEM106B target nucleic acid, such as the gapmer F-G-F′, and may further include 5′ or 3′ nucleosides. The 5′ or 3′ nucleosides may or may not be fully complementary to the TMEM106B target nucleic acid. Such 5′ or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety, the region D′ or D″ can serve as a biocleavable linker. Alternatively, the region D′ or D″ can be used to provide exonuclease protection or for ease of synthesis or manufacture. Region D′ and D″ can be attached to the 5′ end of region F or the 3′ end of region F′, respectively, to generate designs of the following formulas D′-F-G-F′, F-G-F′-D″ or D′-F-G-F′-D″. In this instance, the F-G-F′ is the gapmer portion of the ASO and region D′ or D″ constitute a separate part of the ASO.

Region D′ or D″ may independently include or consist of 1, 2, 3, 4 or 5 additional nucleotides which may be complementary or non-complementary to the TMEM106B target nucleic acid. The nucleotide adjacent to the F or F′ region is not a sugar-modified nucleotide. The D′ or D′ region may serve as a nuclease-susceptible biocleavable linker. In some embodiments the additional 5′ or 3′ end nucleotides are linked with phosphodiester linkages, and are DNA or RNA. Nucleotide-based biocleavable linkers suitable for use as region D′ or D″ are disclosed in WO2014/076195, which include by way of example a phosphodiester linked DNA dinucleotide. The use of biocleavable linkers in poly-ASO constructs is disclosed in WO2015/113922, for use in linking multiple antisense constructs (e.g. gapmer regions) within a single ASO. In some embodiments, the ASO of the disclosure includes a region D′ or D″ in addition to the contiguous nucleotide sequence which constitutes the gapmer.

In some embodiments, the ASO of the present disclosure can be represented by the following formulae: F-G-F′; in particular F₂₋₈-G₆₋₁₆-F′₂₋₈; D′-F-G-F′, in particular D′₂₋₃-F₁₋₈-G₆₋₁₆-F′₂₋₈; F-G-F′-D″, in particular F₂₋₈-G₆₋₁₆-F′₂₋₈-D″₁₋₃; or D′-F-G-F′-D″, in particular D′₁₋₃-F₂₋₈-G₆₋₁₆-F′₂₋₈-D″₁₋₃. In some embodiments, the internucleoside linkage positioned between regions D′ and F is a phosphodiester linkage. In some embodiments the internucleoside linkage positioned between regions F′ and D″ is a phosphodiester linkage.

The term “conjugate,” as used herein, refers to an ASO covalently linked to a non-nucleotide moiety (conjugate moiety, region C, or third region). Conjugation of the ASO of the disclosure to one or more non-nucleotide moieties may improve the pharmacology of the ASO, e.g., by affecting the activity, cellular distribution, cellular uptake, or stability of the ASO. The conjugate moiety may modify or enhance the pharmacokinetic properties of the ASO by improving cellular distribution, bioavailability, metabolism, excretion, permeability, or cellular uptake. In particular, the conjugate may target the ASO to a specific organ, tissue, or cell type, thereby enhancing the effectiveness of the ASO in that organ, tissue, or cell type. At the same time, the conjugate may serve to reduce activity of the ASO in non-target cell types, tissues, or organs, e.g., off target activity or activity in non-target cell types, tissues, or organs. ASO conjugates and their synthesis has also been reported in comprehensive reviews by Manoharan in Antisense Drug Technology, Principles, Strategies, and Applications, S.T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

The non-nucleotide conjugate moiety may be selected from the group consisting of carbohydrates (e.g., GalNAc), cell surface receptor ligands, drug substances, hormones, lipophilic substances, polymers, proteins, peptides, toxins (e.g. bacterial toxins), vitamins, viral proteins (e.g. capsids), or combinations thereof. For example, the conjugate may be an antibody or an antibody fragment which has a specific affinity for a transferrin receptor, for example, as disclosed in WO 2012/143379, which is incorporated by reference herein. In other examples, the non-nucleotide moiety may be an antibody or antibody fragment, such as an antibody or antibody fragment that facilitates delivery across the blood-brain-barrier, such as, e.g., an antibody or antibody fragment targeting the transferrin receptor.

As used herein, a “linkage” or “linker” is a connection between two atoms that links one chemical group or segment of interest to another chemical group or segment of interest via one or more covalent bonds. Conjugate moieties can be attached to the ASO directly or through a linking moiety (e.g. linker or tether). Linkers serve to covalently connect a third region, e.g., a conjugate moiety (region C), to a first region, such as, e.g., an ASO or contiguous nucleotide sequence complementary to the TMEM106B target nucleic acid (region A). The conjugate of the disclosure may optionally include a linker region (second region or region B or region Y) which is positioned between the ASO or contiguous nucleotide sequence thereof (region A or first region) and the conjugate moiety (region C or third region). Region B refers to biocleavable linkers including or consisting of a physiologically labile bond that is cleavable under conditions physiological conditions. Conditions under which physiologically labile linkers undergo chemical cleavage include chemical conditions such as pH, temperature, oxidative or reductive conditions or agents, and physiological salt concentrations. Such physiological conditions may include mammalian intracellular conditions, e.g., the presence of enzymatic activity normally present in a mammalian cell, such as from proteolytic enzymes or hydrolytic enzymes or nucleases. In some embodiments, the biocleavable linker is susceptible to S1 nuclease cleavage. The nuclease susceptible linker may include between 1 and 10 nucleosides, such as 1, 2, 3, 4, 5, 6, 7, 8, 9 or 10 nucleosides, such as, e.g., between 2 and 6 nucleosides, e.g., between 2 and 4 linked nucleosides, and may further include at least two consecutive phosphodiester linkages, such as at least 3, 4, or 5 consecutive phosphodiester linkages. Preferably the nucleosides are naturally-occurring or modified DNA or RNA. Phosphodiester-containing biocleavable linkers are described in more detail in WO 2014/076195 (hereby incorporated by reference).

Region Y refers to linkers that are not necessarily biocleavable but primarily serve to covalently connect a conjugate moiety to an ASO. The region Y linkers may include a chain structure or an oligomer of repeating units such as ethylene glycol, amino acid units, or amino alkyl groups. The ASO conjugates of the present disclosure can be constructed of the following regional elements A-C, A-B-C, A-B-Y-C, A-Y-B-C, or A-Y-C. The linker may be an amino alkyl, such as a C2-C36 amino alkyl group including, e.g., C6 to C12 amino alkyl groups. In some embodiments, the linker is a C6 amino alkyl group.

The term “treatment,” as used herein, refers to both treatment of an existing disease (e.g., a disease or disorder described herein) or prevention of a disease, i.e., prophylaxis. One of skill in the art will recognize that “treatment” may be prophylactic. The treatment may be performed on a patient who has been diagnosed with a neurological disorder, such as a neurological disorder selected from the group consisting of neurodegenerative diseases, including diseases associated with dysregulated TMEM106B expression or activity, Alzheimer's disease (AD), limbic-predominant age-related TDP-43 encephalopathy (LATE), progressive supranuclear palsy (PSP), corticobasal ganglionic degeneration (CBD), chronic traumatic encephalopathy (CTE), frontotemporal dementia FTD), FTD with parkinsonism linked to chromosome 17 (FTDP-17), and Pick's disease (PiD). As used herein, the term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, or N-acetylcysteine. In addition, these salts may be prepared form addition of an inorganic base or an organic base to the free acid. Salts derived from an inorganic base include, but are not limited to sodium, potassium, lithium, ammonium, calcium, or magnesium salts. Salts derived from organic bases include, but are not limited to, salts of primary, secondary, and tertiary amines, substituted amines including naturally-occurring substituted amines, cyclic amines and basic ion exchange resins, such as, e.g., isopropylamine, trimethylamine, diethylamine, triethylamine, tripropylamine, ethanolamine, lysine, arginine, N-ethylpiperidine, piperidine, and polyamine resins. ASO agents disclosed herein may also be present in the form of zwitterions. Some pharmaceutically acceptable salts of ASO compounds of the disclosure may be the salts of hydrochloric acid, hydrobromic acid, sulfuric acid, phosphoric acid, or methanesulfonic acid.

As used herein, the term “protecting group,” alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

### BRIEF DESCRIPTION OF THE DRAWINGS

FIG. 1 shows the structural formula of CMP ID NO: 29_3. Pharmaceutical salts thereof include monovalent cations, such as, Na⁺, K⁺, and Ca²⁺, or a mixture thereof. Figure discloses SEQ ID NO: 29.

FIG. 2 shows the structural formula of CMP ID NO: 29_5. Pharmaceutical salts thereof include monovalent cations, such as, Na⁺, K⁺, and Ca²⁺, or a mixture thereof. Figure discloses SEQ ID NO: 25.

FIG. 3 shows the structural formula of CMP ID NO: 26. Pharmaceutical salts thereof include monovalent cations, such as, Na⁺, K⁺, and Ca²⁺, or a mixture thereof. Figure discloses SEQ ID NO: 19.

The compounds illustrated in FIGS. 1-3 are shown in the protonated form—the S atom on the phosphorothioate linkage is protonated—it will be understood that the presence of the proton will depend on the acidity of the environment of the molecule, and the presence of an alternative cation (e.g., when the antisense oligonucleotide (ASO) is in salt form). Protonated phosphorothioates exist in tautomeric forms.

FIG. 4 is a scatter plot showing the degree of human TMEM106B messenger RNA (mRNA) knockdown induced by 613 new ASO compounds of the disclosure administered to SK-N-BE(2) glioblastoma cells in vitro at a concentration of 2.5 μM for three days. Twenty-two published TMEM106B-targeting ASOs were also tested, including CMP ID NOs: 25_1, 20, and 31 and used as performance (i.e., knockdown) benchmarks for the newly developed compounds of the disclosure. Horizontal, dashed line indicates human TMEM106B mRNA knockdown induced by CMP ID NO 31, a previously published anti-TMEM106B ASO. New compounds CMP ID NOs: 15, 24_3, 12, 29_3, and 26 exhibited greater knockdown relative to CMP ID NO: 31. The x-axis shows nucleotide positions within the human TMEM106B mRNA of SEQ ID NO: 1. The y-axis shows percent knockdown relative to cells treated with a PBS control solution.

FIG. 5 is a scatter plot showing the degree of human TMEM106B mRNA knockdown induced by 908 new ASO compounds of the disclosure administered to SK-N-BE(2) glioblastoma cells in vitro at a concentration of 0.5 μM for three days. Twenty-two published TMEM106B-targeting ASOs were also tested, including CMP ID NOs: 25_1, 20, and 31 and used as performance (i.e., knockdown) benchmarks for the newly developed compounds of the disclosure. Horizontal, dashed line indicates human TMEM106B mRNA knockdown induced by CMP ID NO: 31, a previously published anti-TMEM106B ASO. New compounds CMP ID NOs: 8_1, 8_3, 24_1, 6, 10_4, 29_5, 15, 24_3, 12, 29_3, 26, and 8_4, along with others exhibited greater knockdown relative to CMP ID NO: 31. The x-axis shows nucleotide positions within the human TMEM106B mRNA of SEQ ID NO: 1. The y-axis shows percent knockdown relative to cells treated with a PBS control solution.

FIG. 6 is a scatter plot showing the degree of human TMEM106B mRNA knockdown induced by 312 new ASO compounds of the disclosure administered to SK-N-BE(2) glioblastoma cells in vitro at a concentration of 0.25 μM for three days. Twenty-two published TMEM106B-targeting ASOs were also tested, including CMP ID NO: 25_1 and CMP ID NO: 20 and used as performance (i.e., knockdown) benchmarks for the newly developed compounds of the disclosure. New compounds CMP ID NOs: 8_1, 8_3, 24_1, 6, 8_4, and 29_5, along with others, exhibited greater knockdown relative to CMP ID NO 25_1. The x-axis shows nucleotide positions within the human TMEM106B mRNA of SEQ ID NO: 1. The y-axis shows percent knockdown relative to cells treated with a PBS control solution.

FIG. 7 shows IC50 curves of CMP ID NOs: 29_5, 10_13, and 14. Human iPSC-derived neurons were treated with ASOs of CMP ID NOs: 29_5, 10_13, and 14 in a dose-response curve from 10 to 0.001 μM for seven days in two replicate wells. Taqman assay was used to determine % KD of TMEM106B mRNA level relative to PBS control wells. IC50 curve was determined for each ASO shown in the table (μM).

FIG. 8 shows IC50 curves of CMP ID NOs: 12, 29_3, and 24_3. Human iPSC-derived neurons were treated with ASOs of CMP ID NOs: 12, 29_3, and 24_3 in a dose-response curve from 10 to 0.001 μM for seven days in two replicate wells. Taqman assay was used to determine % KD of TMEM106B mRNA level relative to PBS control wells. IC50 curve was determined for each ASO shown in the table (μM).

FIG. 9 shows IC50 curves of CMP ID NOs: 8_2, 26, and 10_6. Human iPSC-derived neurons were treated with ASOs of CMP ID NOs: 8_2, 26, and 10_6 in a dose-response curve from 10 to 0.001 μM for seven days in two replicate wells. Taqman assay was used to determine % KD of TMEM106B mRNA level relative to PBS control wells. IC50 curve was determined for each ASO shown in the table (μM).

FIGS. 10A-10C show functional assays of ASO compounds CMP ID NOs: 29_3, 15, 26, 14, and control ASO 10_13 human retinal pigment epithelial (RPE) cells. Human RPE cells were treated with ASO of CMP ID NO: 29_3, 15, 26, 14, and control ASO 10_13 for five days in three replicate wells in a concentration range of 10 μM to 0.00001 μM. IC50 curves of indicated ASO are shown in FIG. 10A. At 10 μM, ASO CMP ID NOs: 29_3, 15, and 26 reduced about 80% of TMEM106B mRNA. Human RPE cells were treated with ASO of CMP ID NO: 29_3, 15, 26, 14, and control ASO 10_13 for five days in four replicate wells in a concentration range of 10 μM, 5 μM, and 1 μM and assayed for their effect on lysosomal function using self-quenching bovine serum albumin (BSA)-BODIPY, which fluoresces when proteolytically lysed within the lysosome. The average of BSA degradation activity was normalized to the number of cells per well and then normalized to no ASO-treated wells as control. CMP ID NOs: 29_3, 15, 26, and 14 reduced around lysosomal activity by about 50%. Negative control CMP ID NO: 10_13 did not reduce BSA degradation (n=4, +/−standard error of the mean (SEM)=+/−3; FIG. 10B). Quadruple asterisks denote statistical significance at a level of p<0.0001, double asterisks denote statistical significance at a level of p<0.01, and single asterisks denote statistical significance at a level of p<0.05, as assessed by Dunnett's post-hoc test; ns=not significant. Fluorescence images of human RPE cells (FIG. 10C). White dots show proteolytically cleaved BSA-BODIPY fragments. CMP ID NO: 29_3-treated cells show reduced lysosome activity.

FIG. 11 shows a bar graph of human TMEM106B (hTMEM106B) mRNA measured by qPCR from brain tissue of transgenic mice expressing hTMEM106B treated with an ASO of the disclosure (CMP ID NOs: 14, 15, 29_3, 26, or negative control (NC) ASO of CMP ID NO: 10_13 in vivo. Also shown as a control is hTMEM106B expression from untreated non-transgenic (NTG) mice. The newly developed ASO agents of CMP ID NOs: 29_3 and 26 show superior suppression of hTMEM106B expression in vivo as compared to the prior published ASO of CMP ID NO: 31. Asterisks denote statistical significance at a level of p<0.0001, as assessed by Dunnett's post-hoc test; ns=not significant.

FIG. 12 shows a bar graph of hTMEM106B mRNA measured by qPCR from brain tissue of transgenic mice expressing hTMEM106B treated with an ASO of the disclosure (CMP ID NOs: 8_1, 24_1, 6, 10_4, or the previously published anti-TMEM106B ASO of CMP ID NO: 25_1. Also shown as a control is hTMEM106B expression from saline-treated NTG mice. Asterisks denote statistical significance at a level of p<0.0001, as assessed by Dunnett's post-hoc test; ns=not significant.

FIG. 13 shows a bar graph of hTMEM106B mRNA measured by qPCR from brain tissue of transgenic mice expressing hTMEM106B treated with an ASO of the disclosure (CMP ID NO: 29_3 (25 μg and 100 μg) and CMP ID NO: 26 (25 μg and 100 μg)), the previously published anti-TMEM106B ASO of CMP ID NO: 31, or negative control ASO of CMP ID NO: 10_13 (100 μg) in vivo. Also shown as a control is hTMEM106B expression from non-transgenic mice treated with 100 μg of negative control ASO of CMP ID NO: 10_13. The newly developed ASO agents of CMP ID NOs: 29_3 and 26 show superior suppression of hTMEM106B expression in vivo as compared to the prior published ASO of CMP ID NO: 31. Asterisks denote statistical significance at a level of p<0.0001, as assessed by Dunnett's post-hoc test.

FIG. 14 shows a bar graph of hTMEM106B mRNA measured by qPCR from transgenic mice expressing hTMEM106B treated with an ASO of the disclosure (CMP ID NO: 29_5 (25 μg and 100 μg)), or negative control ASO of CMP ID NO: 13 (100 μg) in vivo. Also shown as a control is hTMEM106B expression from non-transgenic mice treated with 100 μg of negative control ASO of CMP ID NO: 13. Asterisks denote statistical significance at a level of ***p<0.001 or ****p<0.0001, as assessed by Dunnett's post-hoc test.

### DETAILED DESCRIPTION

Described herein are antisense oligonucleotide (ASO) agents that target, bind to, and promote the degradation of Transmembrane Protein 106B (TMEM106B) mRNA, and pharmaceutically acceptable compositions containing the same. Such agents and compositions may be particularly useful for treatment of neurodegenerative disorders (e.g., frontotemporal lobar degeneration (FTD), Parkinson's disease (PD), Alzheimer's disease (AD), hippocampal sclerosis, hypomyelinating leukodystrophy (HML), amyotrophic lateral sclerosis (ALS), corticobasal syndrome (CBS), limbic-predominant age-related TDP-43 encephalopathy (LATE), progressive supranuclear palsy (PSP), and neuronal ceroid lipofuscinosis (NCL)). For example, a subject (e.g., human) having or at risk of developing a neurodegenerative disorder may be administered one or more ASO agents or compositions containing the same by any route in order to treat the disorder in the subject. The present disclosure encompasses treatment of any disease or condition that is associated with increased levels of TMEM106B and would benefit from a reduction of the protein in the subject.

### Transmembrane Protein 106B

Transmembrane protein 106B (TMEM106B) is a single-pass, type 2 integral membrane glycoprotein predominantly located in the membranes of endosomes and lysosomes. It is expressed in neurons as well as glial and endothelial cells. It is believed to be primarily involved in lysosomal functioning. Overexpression and knock down of TMEM106B in cell lines can cause an increase in lysosomal size, number, and acidification. TMEM106B has been associated with several common neurodegenerative disorders including frontotemporal lobar degeneration (FTLD) (Nicholson and Rademakers, Acta Neuropathol. 132:639-651 (2016)).

Neurodegenerative disorders, including, e.g., frontotemporal lobar degeneration (FTLD), Parkinson's disease (or parkinsonism), hypomyelinating leukodystrophies, amyotrophic lateral sclerosis (ALS), Alzheimer's disease, hippocampal sclerosis, aging, Alzheimer's Disease (AD), corticobasal syndrome, limbic-predominant age-related TDP-43 encephalopathy (LATE), progressive supranuclear palsy (PSP), and neuronal ceroid lipofuscinosis (NCL), represent a major unmet medical need, and there is clear genetic and experimental evidence which indicates specific TMEM106B allele variants and altered TMEM106B expression in neurodegenerative disorders. There is therefore a need for agents that inhibit the expression of TMEM106B for use in research and therapeutic applications.

### Neurodegenerative Disorders

Neurodegenerative disorders may be associated with dysregulated TMEM106B expression or activity. Exemplary disorders associated with dysregulated expression of TMEM106B may include neurodegenerative disorders such as, e.g., frontotemporal lobar degeneration (FTLD), Parkinson's disease (or parkinsonism; PD), FTD with parkinsonism linked to chromosome 17 (FTDP-17), hypomyelinating leukodystrophies (HML), amyotrophic lateral sclerosis (ALS), Alzheimer's disease (AD), hippocampal sclerosis, corticobasal ganglionic degeneration (CBD), corticobasal syndrome (CBS), chronic traumatic encephalopathy (CTE), Pick's disease (PiD), limbic-predominant age-related TDP-43 encephalopathy (LATE), progressive supranuclear palsy (PSP), and neuronal ceroid lipofuscinosis (NCL). Given that several of these disorders are associated with dysfunction of the frontal cortex, the compounds of the disclosure may be used for the treatment of age-associated or disease-associated changes in frontal cortex.

Accordingly, ASOs described herein may be suitable for use in the treatment of FTLD. In particular, the ASOs or pharmaceutical compositions of the disclosure may be advantageous in the treatment of FTLD characterized by intranuclear or cytoplasmic accumulations of ubiquitinated proteins (FTLD-U), such as, e.g., FTLD-TDP, which is characterized by the presence of ubiquitinated TAR DNA binding protein 43 (TDP-43) deposits in frontal and temporal brain regions, as well as other TDP-43 proteinopathies (e.g., ALS, Alzheimer's disease, cerebral age-related TDP-43 with sclerosis (CARTS), limbic-predominant age-related TDP-43 encephalopathy (LATE), chronic traumatic encephalopathy (CTE), limbic-predominant age-related TDP-43 encephalopathy (LATE), and hippocampal sclerosis).

### Frontotemporal Lobar Degeneration

FTLD is a clinical syndrome characterized by progressive neurodegeneration in the frontal and temporal lobes of the cerebral cortex. The manifestation of FTLD is complex and heterogeneous, and may present as one of three clinically distinct variants including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe; 2) semantic dementia (SD), characterized by fluent, anomic aphasia, progressive loss of semantic knowledge of words, objects, and concepts and a pronounced degeneration of the anterior temporal lobes; or 3) progressive nonfluent aphasia (PNA); characterized by motor deficits in speech production, reduced language expression, and pronounced degeneration of the perisylvian cortex. Neuronal loss in brains of FTLD patients is associated with one of three distinct neuropathologies: 1) the presence of tau-positive neuronal and glial inclusions; 2) ubiquitin (ub)-positive and TAR DNA-binding protein 43 (TDP43)-positive, but tau-negative inclusions; or 3) ub and fused in sarcoma (FUS)-positive, but tau and TDP-43-negative inclusions. These neuropathologies are considered to be important in the etiology of FTLD.

Nearly half of FTLD patients have a first-degree family member with dementia, ALS, or Parkinson's disease, suggesting a strong genetic link to the cause of the disease. A number of mutations in chromosome 17q21 have been linked to FTLD presentation.

### Alzheimer's Disease

Alzheimer's disease is a neurodegenerative disorder characterized by progressive neuronal loss in the frontal, temporal, and parietal lobes of the cerebral cortex as well as subcortical structures like the basal forebrain cholinergic system and the locus coeruleus within the brainstem. The clinical presentation of Alzheimer's disease is a progressive decline in a number of cognitive functions including short and long-term memory, spatial navigation, language fluency, impulse control, anhedonia, and social withdrawal. Neuronal atrophy in brains of Alzheimer's disease patients is linked to accumulation of extracellular and intracellular protein inclusions. Aggregates of insoluble amyloid-β (Aβ) protein are often found in the extracellular space, while neurofibrillary tangles (NFTs) of hyperphosphorylated tau proteins are usually found in intracellular compartments of affected neurons. These neuropathologies are considered to be important in the etiology of Alzheimer's disease.

Clinical management of Alzheimer's disease has employed pharmacological and behavioral interventions to mitigate the symptoms of the disorder. For example, acetylcholinesterase inhibitors have been used to elevate acetylcholine levels in the brain as a means to ameliorate cognitive deficits of Alzheimer's disease as this neurotransmitter is found to be depleted in Alzheimer's disease patients. Additionally, atypical antipsychotics are commonly prescribed to Alzheimer's disease patients for behavioral management. This strategy, however, is targeted at ameliorating the symptoms of the disease without addressing its development and progression. Unlike these treatments, the compositions and methods described herein provide the benefit of treating a different biochemical phenomenon that can underlie the development of Alzheimer's disease. As such, the compositions and methods described herein target the physiological cause of the disease, representing a potential curative therapy.

### Hypomyelinating Leukodystrophy

Hypomyelinating leukodystrophy refers to a set of predominately inherited disorders that feature degeneration of the white matter in the brain. Hypomyelinating leukodystrophy may be characterized by the presence of white matter degeneration, loss of axons and myelin, presence of axonal spheroids, and, in some cases, cystic bone lesions in the distal extremities (e.g., Nasu-Hakola disease). Leukodystrophies generally present around infancy and early childhood and may be characterized by hyperirritability, hypersensitivity to the environment, muscle rigidity, backwards-bent head, decrease or loss of hearing and vision, and epilepsy. Non-limiting examples of leukodystrophies include Nasu-Hakola disease (PLOSL), metachromatic leukodystrophy, Krabbe disease, X-linked adrenoleukodystrophy, Canavan disease, and Alexander disease.

### Amyotrophic Lateral Sclerosis

ALS, also known as Lou Gehrig's disease, is the most fatal neurodegenerative disorder which features loss of motor neurons in primary motor cortex, the brainstem, and the spinal cord. The loss of motor neurons results in profound impairments in rudimentary motor control, including breathing and results in death within 2-5 years after diagnosis. ALS patients typically require respiratory support at advanced stages of the disease. These patients also present with muscle weakness in striated muscle as well as smooth muscle, e.g., muscles of swallowing. Some patients may also contemporaneously develop frontotemporal dementia.

Two general forms of ALS have been described, namely sporadic ALS (sALS), which is the most common form of ALS in the U.S. and accounts for 90 to 95% of all cases diagnosed, and familial ALS (fALS), which exhibits dominant inheritance and accounts for about 5 to 10% of all cases in the U.S. These two forms of ALS are clinically indistinguishable.

At the molecular level, ALS is associated with disrupted cellular processes occur following disease onset, including, e.g., ER stress, production of reactive oxygen species, mitochondrial dysfunction, protein aggregation, apoptosis, inflammation, and glutamate excitotoxicity, particularly in motor neurons. Despite these findings, the causes of ALS are complex and heterogenous. In general, ALS is considered to be a complex genetic disorder in which multiple genes interact with environmental factors to increase susceptibility to the disease. Over a dozen genes have been associated with ALS pathology, including Cu²⁺/Zn²⁺ superoxide dismutase (SOD-1), TARDBP, TAR DNA binding protein-43 (TDP-43), Fused in Sarcoma/Translocated in Sarcoma (FUS), Angiogenin (ANG), Ataxin-2 (ATXN2), valosin containing protein (VCP), Optineurin (OPTN), and an expansion of the noncoding GGGGCC hexanucleotide repeat in the chromosome 9, open reading frame 72 (C9ORF72). However, the precise mechanisms of motor neuron degeneration remain unknown.

No curative treatments for ALS are presently available. The only FDA-approved drug is Riluzole, an antagonist of TTX-sensitive sodium channels, kainate, and NMDA receptors, and a potentiator of GABAa receptors. However, only about a three-month life span extension for ALS patients in the early stages has been reported, and no therapeutic benefit for ALS patients in the late stages has been observed.

### Multiple System Atrophy

MSA is a neurodegenerative disorder that typically presents with dysregulated autonomic function, tremors, hypokinesia, muscle rigidity, parkinsonism and ataxia. Patients with MSA generally live for 6-10 years following diagnosis. At the cellular level, MSA corresponds to insidious loss of neurons within the basal ganglia, inferior olivary nucleus, and cerebellum. The presence of a modified form of alpha-synuclein has been postulated to be a potential cause for MSA. Lewy bodies have also been found in brain tissue from MSA patients. Another common feature of MSA is gliosis, proliferation of astrocytes, glial scars, Papp-Lantos bodies made up of alpha-synuclein, and tau protein.

Onset of MSA is typically associated with presentation of akinetic-rigid syndrome (62% of cases), as well as disturbed balance, genito-urinary symptoms, and falls. Disease progression is characterized by progressive parkinsonism, urinary incontinence or retention, impotence, constipation, vocal cord paralysis, dry mouth and skin, thermal dysregulation, breathing problems, vision problems, and cognitive impairment.

MSA is generally divided into two subtypes, namely MSA with predominant parkinsonism (MSA-P) and MSA with cerebellar features (MSA-C). MSA-P features extrapyramidal motor impairments and may include degeneration in the striatonigral system. MSA-C features cerebellar ataxia and may be associated with sporadic olivopontocerebellar atrophy.

Treatment of MSA has been largely palliative and no curative therapies have yet been developed to treat this devastating disorder.

### Motor Neuron Disease

MND belongs to a group of neurological disorders attributed to the destruction of motor neurons of the CNS and degenerative changes in the motor neuron circuits. MNDs are typically progressive, degenerative disorders that affect upper and lower motor neurons, leading to successive global muscular denervation. MNDs often present in middle age, although a wide age range of symptomatic disease onset has been observed from age 18 to 85. MND symptoms often include difficulty swallowing, limb weakness, slurred speech, impaired gait, weakness of facial muscles, and muscle cramps. At advanced stages of the disease, the respiratory musculature loses its motor innervation, which is usually the final cause of death of ALS patients. The etiology of most MNDs is unknown, but environmental, toxic, viral, and genetic factors have been attributed to disease pathology.

Motor neurons, including upper motor neurons and lower motor neurons, affect voluntary muscles, stimulating them to contract. Upper motor neurons originate in the cerebral cortex and send axon fibers through the brainstem and the spinal cord to control lower motor neurons. Lower motor neurons are located in the brainstem and the spinal cord and innervate the musculoskeletal system. Lower motor neuron diseases are diseases involving lower motor neuron degeneration. Lower motor neuron degeneration is associated with disconnection of neurons and muscle, leading to muscle weakness and diminished reflexes. Loss of upper or lower motor neurons results in weakness, muscle atrophy (wasting), and painless weakness are the clinical hallmarks of MND.

### Corticobasal Syndrome

Corticobasal syndrome is a progressive atypical Parkinsonism syndrome associated with tauopathy and FTLD. Corticobasal syndrome can present in one of three forms, namely frontal-behavioral dysexecutive-spatial syndrome (FBS), nonfluent/agrammatic variant of primary progressive aphasia, and progressive supranuclear palsy syndrome (PSPS). Common symptoms generally include apraxia, alien limb phenomenon, frontal deficits, and extrapyramidal motor symptoms, including myoclonus or rigidity. Corticobasal syndrome normally leads to death within 6.5 years following diagnosis. No curative treatments are presently available for this condition.

### Progressive Supranuclear Palsy

PSP is a progressive neurodegenerative disease characterized by falls, vertical ophthalmoparesis, akinetic-rigid features, prominent bulbar dysfunction, and fronto-subcortical dementia. The loss of independent gait—the inability to stand unassisted—occurs less than 5 years after disease onset. PSP is associated with profound neuronal loss in the substantia nigra, globus pallidus, subthalamic nucleus, midbrain, and pontine reticular formation, and the presence of neurofibrillary tangles composed of tau filaments. In addition to the presence of extensive and multifocal neuropathological changes, there are also multiple neurotransmitter abnormalities, including dysfunction of dopamine, acetylcholine, GABA and the noradrenaline systems.

The name “progressive supranuclear palsy” describes the main feature of the disease the progressive failure of arbitrary eye movements. The onset of the disease is usually between the age of 50 and 70 years. Many patients report initially to have a constant vertigo and balance problems or constant falls, typically backwards. The reduction of the arbitrary eye movements reduces the capability to read, climb stairs, and drive motor vehicles. Additional early symptoms may include personality changes, such as, e.g., irritability or loss of impulse control. Some patients lose the interest in daily activities and hobbies. Even in the early phase of the disease mood changes and depression are very common.

The regions of the brain which control the eye movements are located close to the regions which control the tongue and muscles for swallowing. The speech of the patients is usually slowed and deepened. Swallowing of liquids and food is also impaired as the disease progresses, which leads to life-threatening pneumonias. This is the main cause of death in advanced PSP. To date, there is no curative treatment for the disease.

### Neuronal Ceroid Lipofuscinosis

NCL is an umbrella term that relates to a collection of at least eight clinically recognized lysosomal storage disorders caused by the accumulations of lipofuscin within cells of the body, such as neuronal, liver, spleen, myocardium, and kidney cells. NCL clinically presents with profound neurodegeneration and progressive and irreversible loss of motor and cognitive abilities, although the disease severity and clinical presentation may depend on the particular NCL variant.

Known variants of NCL include the infantile variant, also known as Santovuori-Haltia disease (SHD), the late infantile variant known as Jansky-Bielschowsky disease (JBD), the Finnish late infantile variant (FLI), the variant late infantile (VLI), the CLN7 variant (CLN7), the CLN8 variant (CLN8), the Turkish late infantile variant (TLI), the type 9 variant (T9), the CLN10 variant (CLN10), the juvenile variant also known as Batten disease (BD), and the adult variant also known as Kuf's disease (KD). SHD is associated with early visual loss that progressively turns to complete retinal blindness by the age of 2, followed by a vegetative state at 3 years, and brain death by year 4. This variant is also associated with the spontaneous occurrence of epileptic seizures. The JBD variant emerges between ages 2 to 4 and is associated with ataxia, epileptic seizures, progressive cognitive decline, and abnormal speech development and typically results in death by age 8. BD typically emerges between 4 and 10 years of age and include symptoms such as vision loss, epileptic seizures, cognitive dysfunction, and premature death. NCL patients having the KD variant generally present with milder symptoms than SHD and BD variants and have a life expectancy of around 40 years.

### Antisense Oligonucleotides

The disclosure relates to ASO compounds capable of modulating expression of TMEM106B, such as inhibiting TMEM106B. The modulation is achieved by contacting a target nucleic acid (e.g., pre-mRNA or mRNA) encoding TMEM106B with an ASO capable of hybridizing to the TMEM106B target nucleic acid and promoting its degradation by promoting recruitment of RNase H and by inhibition of translation (e.g., through steric hinderance). The TMEM106B target nucleic acid may be a mammalian TMEM106B mRNA sequence, such as a sequence selected from the group consisting of SEQ ID NOs: 1-4.

The ASO compounds of the disclosure include a nucleoside sequence that is at least substantially complementary or fully complementary to a region of the sequence of TMEM106B mRNA (e.g., any one of SEQ ID NOs: 1-4) or variants thereof, said complementarity being sufficient to yield specific binding under intracellular conditions. For example, the disclosure contemplates an ASO having an antisense sequence that is complementary to at least 7 (e.g., at least 7, 8, 9, 10, 11, 12, 13, 14, 15, 16, 17, 18, 19, 20, 21, 22, or more) consecutive nucleotides of one or more regions of a TMEM106B mRNA. In a particular example, the ASO has an antisense sequence that is complementary to 7 consecutive nucleotides of one or more regions of a TMEM106B mRNA. In another example, the ASO has an antisense sequence that is complementary to 8 consecutive nucleotides of one or more regions of a TMEM106B mRNA. In another example, the ASO has an antisense sequence that is complementary to 9 consecutive nucleotides of one or more regions of a TMEM106B mRNA. In another example, the ASO has an antisense sequence that is complementary to 10 consecutive nucleotides of one or more regions of a TMEM106B mRNA. In another example, the ASO has an antisense sequence that is complementary to 11 consecutive nucleotides of one or more regions of a TMEM106B mRNA. In another example, the ASO has an antisense sequence that is complementary to 12 consecutive nucleotides of one or more regions of a TMEM106B mRNA. In another example, the ASO has an antisense sequence that is complementary to 13 consecutive nucleotides of one or more regions of a TMEM106B mRNA. In another example, the ASO has an antisense sequence that is complementary to 14 consecutive nucleotides of one or more regions of a TMEM106B mRNA. In another example, the ASO has an antisense sequence that is complementary to 15 consecutive nucleotides of one or more regions of a TMEM106B mRNA. In another example, the ASO has an antisense sequence that is complementary to 16 consecutive nucleotides of one or more regions of a TMEM106B mRNA. In another example, the ASO has an antisense sequence that is complementary to 17 consecutive nucleotides of one or more regions of a TMEM106B mRNA. In another example, the ASO has an antisense sequence that is complementary to 18 consecutive nucleotides of one or more regions of a TMEM106B mRNA. In another example, the ASO has an antisense sequence that is complementary to 19 consecutive nucleotides of one or more regions of a TMEM106B mRNA. In another example, the ASO has an antisense sequence that is complementary to 20 consecutive nucleotides of one or more regions of a TMEM106B mRNA. In another example, the ASO has an antisense sequence that is complementary to 21 consecutive nucleotides of one or more regions of a TMEM106B mRNA. In another example, the ASO has an antisense sequence that is complementary to 22 consecutive nucleotides of one or more regions of a TMEM106B mRNA. In another example, the ASO has an antisense sequence that is complementary to 23 consecutive nucleotides of one or more regions of a TMEM106B mRNA. In another example, the ASO has an antisense sequence that is complementary to 24 consecutive nucleotides of one or more regions of a TMEM106B mRNA. In another example, the ASO has an antisense sequence that is complementary to 25 consecutive nucleotides of one or more regions of a TMEM106B mRNA. In another example, the ASO has an antisense sequence that is complementary to 26 consecutive nucleotides of one or more regions of a TMEM106B mRNA. In another example, the ASO has an antisense sequence that is complementary to 27 consecutive nucleotides of one or more regions of a TMEM106B mRNA. In another example, the ASO has an antisense sequence that is complementary to 28 consecutive nucleotides of one or more regions of a TMEM106B mRNA. In another example, the ASO has an antisense sequence that is complementary to 29 consecutive nucleotides of one or more regions of a TMEM106B mRNA. In another example, the ASO has an antisense sequence that is complementary to 30 consecutive nucleotides of one or more regions of a TMEM106B mRNA. In yet another example, the ASO has an antisense sequence that is 100% complementary to the nucleotides of one or more regions of a TMEM106B mRNA.

The disclosure contemplates ASO compounds that, when bound to one or more regions of a TMEM106B mRNA (e.g., any one of the regions of TMEM106B mRNA described in SEQ ID NOs: 1-4), form a duplex structure with the TMEM106B mRNA of between 7-22 (e.g., 7, 8, 9, 10, 11, 12, 13, 14, 15, 16, 17, 18, 19, 20, 21, or 22) nucleotides in length. For example, the duplex structure between the ASO compound and the TMEM106B mRNA may be 7 nucleotides in length. In another example, the duplex structure between the ASO compound and the TMEM106B mRNA may be 8 nucleotides in length. In another example, the duplex structure between the ASO compound and the TMEM106B mRNA may be 9 nucleotides in length. In another example, the duplex structure between the ASO compound and the TMEM106B mRNA may be 10 nucleotides in length. In another example, the duplex structure between the ASO compound and the TMEM106B mRNA may be 11 nucleotides in length. In another example, the duplex structure between the ASO compound and the TMEM106B mRNA may be 12 nucleotides in length. In another example, the duplex structure between the ASO compound and the TMEM106B mRNA may be 13 nucleotides in length. In another example, the duplex structure between the ASO compound and the TMEM106B mRNA may be 14 nucleotides in length. In another example, the duplex structure between the ASO compound and the TMEM106B mRNA may be 15 nucleotides in length. In another example, the duplex structure between the ASO compound and the TMEM106B mRNA may be 16 nucleotides in length. In another example, the duplex structure between the ASO compound and the TMEM106B mRNA may be 17 nucleotides in length. In another example, the duplex structure between the ASO compound and the TMEM106B mRNA may be 18 nucleotides in length. In another example, the duplex structure between the ASO compound and the TMEM106B mRNA may be 19 nucleotides in length. In another example, the duplex structure between the ASO compound and the TMEM106B mRNA may be 20 nucleotides in length. In another example, the duplex structure between the ASO compound and the TMEM106B mRNA may be 21 nucleotides in length. In yet another example, the duplex structure between the ASO compound and the TMEM106B mRNA may be 10 nucleotides in length.

According to the disclosed methods and compositions, the duplex structure formed by an ASO (e.g., an agent having at least 90% (at least 91%, 92%, 93%, 94%, 95%, 96%, 97%, 98%, 99%, or more) sequence identity to the nucleic acid sequence of any one of SEQ ID NOs: 5-50) and one or more regions of a TMEM107B mRNA may include at least one (e.g., at least 2, 3, 4, 5, 6, 7, 8, 9, 10, 11, 12, 13, 14, or 15) mismatch, where a mismatch may be an insertion of a nucleobase that is not present in the TMEM107B mRNA, a deletion of a nucleobase that is present in the TMEM106B mRNA, or a nucleobase that is not complementary to the TMEM106B mRNA. For example, the duplex structure may contain 1 mismatch. In another example, the duplex structure contains 2 mismatches. In another example, the duplex structure contains 3 mismatches. In another example, the duplex structure contains 4 mismatches. In another example, the duplex structure contains 5 mismatches. In another example, the duplex structure contains 6 mismatches. In another example, the duplex structure contains 7 mismatches. In another example, the duplex structure contains 8 mismatches. In another example, the duplex structure contains 9 mismatches. In another example, the duplex structure contains 10 mismatches. In another example, the duplex structure contains 11 mismatches. In another example, the duplex structure contains 12 mismatches. In another example, the duplex structure contains 13 mismatches. In another example, the duplex structure contains 14 mismatches. In yet another example, the duplex structure contains 15 mismatches.

In some embodiments, the TMEM106B target nucleic acid is a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 1. In some embodiments, the TMEM106B target nucleic acid is a polynucleotide having at least 90% (e.g., at least 91%, 92%, 93%, 94%, 95%, 96%, 97%, 98%, 99%, or more) sequence identity to the nucleic acid sequence of SEQ ID NO: 1. In some embodiments, the TMEM106B target nucleic acid is a polynucleotide having at least 95% (e.g., at least 96%, 97%, 98%, 99%, or more) sequence identity to the nucleic acid sequence of SEQ ID NO: 1. In some embodiments, the TMEM106B target nucleic acid is a polynucleotide having the nucleic acid sequence of SEQ ID NO: 1.

In some embodiments, the TMEM106B target nucleic acid is a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 2. In some embodiments, the TMEM106B target nucleic acid is a polynucleotide having at least 90% (e.g., at least 91%, 92%, 93%, 94%, 95%, 96%, 97%, 98%, 99%, or more) sequence identity to the nucleic acid sequence of SEQ ID NO: 2. In some embodiments, the TMEM106B target nucleic acid is a polynucleotide having at least 95% (e.g., at least 96%, 97%, 98%, 99%, or more) sequence identity to the nucleic acid sequence of SEQ ID NO: 2. In some embodiments, the TMEM106B target nucleic acid is a polynucleotide having the nucleic acid sequence of SEQ ID NO: 2.

In some embodiments, the TMEM106B target nucleic acid is a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 3. In some embodiments, the TMEM106B target nucleic acid is a polynucleotide having at least 90% (e.g., at least 91%, 92%, 93%, 94%, 95%, 96%, 97%, 98%, 99%, or more) sequence identity to the nucleic acid sequence of SEQ ID NO: 3. In some embodiments, the TMEM106B target nucleic acid is a polynucleotide having at least 95% (e.g., at least 96%, 97%, 98%, 99%, or more) sequence identity to the nucleic acid sequence of SEQ ID NO: 3. In some embodiments, the TMEM106B target nucleic acid is a polynucleotide having the nucleic acid sequence of SEQ ID NO: 3.

In some embodiments, the TMEM106B target nucleic acid is a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 4. In some embodiments, the TMEM106B target nucleic acid is a polynucleotide having at least 90% (e.g., at least 91%, 92%, 93%, 94%, 95%, 96%, 97%, 98%, 99%, or more) sequence identity to the nucleic acid sequence of SEQ ID NO: 4. In some embodiments, the TMEM106B target nucleic acid is a polynucleotide having at least 95% (e.g., at least 96%, 97%, 98%, 99%, or more) sequence identity to the nucleic acid sequence of SEQ ID NO: 4. In some embodiments, the TMEM106B target nucleic acid is a polynucleotide having the nucleic acid sequence of SEQ ID NO: 4.

In some embodiments, the ASO of the disclosure is capable of modulating the expression of the TMEM106B target nucleic acid by inhibiting or downregulating the expression of the TMEM106B target nucleic acid (TMEM106B pre-mRNA or mRNA) in a cell. Such modulation produces an inhibition of expression of TMEM106B target nucleic acid of at least 10% (e.g., at least 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) compared to the expression level of the target in the cell in the absence of treatment with the ASO. The modulation of TMEM106B expression may occur in vitro (e.g., in primary cell cultures, cell lines, or tissue organoids) or in vivo (e.g., upon administration to an animal, e.g., a mammal, e.g., a human). In some embodiments, ASOs of the disclosure may be capable of inhibiting expression levels of TMEM106B mRNA in a primary neuronal cell by at least 60% (e.g., at least 65%, 70%, 75%, 80%, 85%, 90%, 91%, 92%, 93%, 94%, 95%, 96%, 97%, 98%, 99% or more) in vitro following application of 5 μM ASO to primary neuronal cells. In some embodiments, ASOs of the disclosure may be capable of inhibiting expression levels of TMEM106B protein in a primary neuronal cell by at least 50% (e.g., at least 55%, 60%, 65%, 70%, 75%, 80%, 85%, 90%, 91%, 92%, 93%, 94%, 95%, 96%, 97%, 98%, 99% or more) in vitro following application of 0.5 μM ASO to primary neuronal cells. Suitably, the examples provide assays, which may be used to measure TMEM106B RNA or protein inhibition (e.g., Example 1, 2, and 5).

The modulation of the TMEM106B target nucleic acid may be triggered by the hybridization between a contiguous nucleotide sequence of the ASO and the TMEM106B target nucleic acid. In some embodiments, the ASO of the disclosure includes one or more (e.g., 1, 2, 3, 4, 5, 6, 7, 8, 10, 11, 12, 13, 14, 15, or more) mismatches between the ASO and the TMEM106B target nucleic acid. Hybridization to the TMEM106B target nucleic acid may still be sufficient to show a desired modulation of TMEM106B expression even in the absence of the one or more mismatches. Reduced binding affinity resulting from mismatches may advantageously be compensated by increasing number of nucleotides in the ASO or increasing the number of modified nucleosides capable of increasing the binding affinity to the target, such as, e.g., 2′ sugar-modified nucleosides (e.g., LNA) present within the ASO sequence.

In some embodiments, the ASO includes a contiguous sequence of 10 to 30 nucleotides in length, which is at least 90% (e.g., at least 91%, 92%, 93%, 94%, 95%, 96%, 97%, 98%, 99%, or more) complementary to a region of the TMEM106B target nucleic acid. The ASO of the disclosure, or contiguous nucleotide sequence thereof, may be fully complementary (i.e., 100% complementary) to a region of the TMEM106B target nucleic acid.

The ASO of the disclosure may include or consists of 10 to 35 nucleotides in length (e.g., 10, 11, 12, 13, 14, 15, 16, 17, 18, 19, 20, 21, 22, 23, 24, 25, 26, 27, 28, 29, 30, 31, 32, 33, 34, or 35 nucleotides in length). In some embodiments, the ASO includes or consists of 16 to 22 (e.g., 16, 17, 18, 19, 20, 21, or 22) nucleotides in length. In some embodiments, the ASO includes or consists of 16 to 20 (e.g., 16, 17, 18, 19, 20) nucleotides in length. In some embodiments, the ASO or contiguous nucleotide sequence thereof includes or consists of 22 nucleotides or less (e.g., 22, 21, 20, 19, 18, 17, 16 nucleotides, or less). It is to be understood that any range given herein includes the range endpoints. For example, if an ASO is said to include from 10 to 30 nucleotides, both 10 and 30 nucleotides are included. In some embodiments, the contiguous nucleotide sequence includes or consists of 10, 11, 12, 13, 14, 15, 16, 17, 18, 19, 20, 21, 22, 23, 24, 25, 26, 27, 28, 29 or 30 contiguous nucleotides. In some embodiments, the ASO includes or consists of 16, 17, 18, 19 or 20 nucleotides.

In some embodiments, the ASO or contiguous nucleotide sequence includes or consists of 10 to 30 nucleotides in length with at least 90% (e.g., at least 91%, 92%, 93%, 94%, 95%, 96%, 97%, 98%, 99%, or more) identity to a sequence selected from the group consisting of SEQ ID NOs: 5-50—(see motif sequences listed in Table 5 of the Materials and Method section). In some embodiments, the ASO or contiguous nucleotide sequence includes or consists of 10 to 30 (e.g., 10, 11, 12, 13, 14, 15, 16, 17, 18, 19, 20, 21, 22, 23, 24, 25, 26, 27, 28, 29, or 30) nucleotides in length with at least 90% identity, preferably 100% identity, to a sequence selected from the group consisting of SEQ ID NOs: 5-50 (see motif sequences listed in Table 5). In some embodiments, the ASO or contiguous nucleotide sequence includes or consists of 10 to 30 nucleotides in length with at least 90% (e.g., at least 91%, 92%, 93%, 94%, 95%, 96%, 97%, 98%, 99%, or more) complementarity to a sequence of SEQ ID NOs: 5-50 (see target sequences listed in Table 5).

It is understood that the contiguous nucleobase sequences can be modified to, e.g., increase nuclease resistance or binding affinity to the TMEM106B target nucleic acid. The pattern in which the modified nucleosides (such as high affinity modified nucleosides) are incorporated into the ASO sequence is generally termed ASO design. The ASOs of the disclosure are designed with modified nucleosides and DNA nucleosides. Advantageously, high affinity modified nucleosides are used.

In some embodiments, the ASO includes at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In some embodiments, the ASO includes from 1 to 10 (e.g., 1, 2, 3, 4, 5, 6, 7, 8, 9, or 10) modified nucleosides. Suitable modifications are described in the “Definitions” section under “modified nucleoside,” “high affinity modified nucleosides,” “sugar modifications,” “2′ sugar modifications,” Locked nucleic acids (LNA),” “inverted nucleotides,” among others.

In some embodiments, the ASO includes one or more sugar-modified nucleosides, such as 2′ sugar-modified nucleosides. Preferably the ASO of the disclosure includes one or more (e.g., 1, 2, 3, 4, 5, 6, 7, 8, 9, 10, 11, 12, 13, 14, 15, or more) 2′ sugar-modified nucleoside independently selected from the group consisting of 2′-O-alkyl-RNA (e.g., 2′-O-methyl-RNA), 2′-alkoxy-RNA, 2′-O-methoxyethyl-RNA, 2′-amino-DNA, 2′-fluoro-DNA, arabino nucleic acid (ANA), 2′-fluoro-ANA, and LNA nucleosides. It is advantageous if one or more (e.g., 1, 2, 3, 4, 5, 6, 7, 8, 9, 10, 11, 12, 13, 14, 15, or more) of the modified nucleosides is a locked nucleic acid (LNA).

In some embodiments, the ASO of the disclosure includes at least one LNA nucleoside, such as at least 1, 2, 3, 4, 5, 6, 7, or 8 LNA nucleosides, such as from 2 to 6 (e.g., 2, 3, 4, 5, or 6) LNA nucleosides, such as from 3 to 7 (e.g., 3, 4, 5, 6, or 7) LNA nucleosides, 4 to 8 (e.g., 4, 5, 6, 7, or 8) LNA nucleosides or 3, 4, 5, 6, 7 or 8 LNA nucleosides. In some embodiments, at least 75% (e.g., at least 76%, 80%, 85%, 90%, 95%, 96%, 97%, 98%, 99%, or more) of the modified nucleosides in the ASO are LNA nucleosides, such as, e.g., beta-D-oxy LNA or ScET. In some embodiments, all the modified nucleosides in the ASO are LNA nucleosides. In some embodiments, the ASO may include beta-D-oxy-LNA and one or more (e.g., 1, 2, 3, 4, 5, 6, 7, 8, 9, 10, 11, 12, 13, 14, 15, or more) of the following LNA nucleosides: thio-LNA, amino-LNA, oxy-LNA, ScET, or ENA in either the beta-D or alpha-L configurations or combinations thereof. In some embodiments, all of the LNA cytosine nucleosides are 5-methylcytosine nucleosides. The ASO or contiguous nucleotide sequence may have at least 1 LNA nucleoside at the 5′ end and at least 2 LNA nucleosides at the 3′ end of the nucleotide sequence to impart nuclease resistance to the ASO. In some embodiments, an ASO of the disclosure is capable of recruiting RNase H.

In some embodiments, the ASO may include one or more (e.g., 1, 2, 3, 4, 5, 6, 7, 8, 9, 10, 11, 12, 13, 14, 15, or more) inverted nucleosides (see Materials and Methods). In some embodiments, the ASO may include one or more (e.g., 1, 2, 3, 4, 5, 6, 7, 8, 9, 10, 11, 12, 13, 14, 15, or more) DNA 7-deaza-8-aza-guanine pyrazolo[3,4-d]pyrimidine (PPG) nucleoside.

Within the context of the disclosure, it may be advantageous to employ an ASO structural design that is a gapmer design as described in the “Definitions” section under for example “Gapmer,” “LNA Gapmer,” “MOE gapmer,” “Mixed Wing Gapmer,” and “Alternating Flank Gapmer”. The gapmer design includes gapmers with uniform flanks, mixed wing flanks, alternating flanks, and gap-breaker designs. It may be advantageous if the ASO of the disclosure is a gapmer with an F-G-F′ design, a particular gapmer of formula 5′-F-G-F′-3′, where regions F and F′ independently include 1-8 (e.g., 1, 2, 3, 4, 5, 6, 7, or 8) nucleosides, of which 2-5 (e.g., 2, 3, 4, or 5) are 2′ sugar-modified and define the 5′ and 3′ end of the F and F′ region, and G is a region between 6-16 (e.g., 6, 7, 8, 9, 10, 11, 12, 13, 14, 15, or 16) nucleosides which are capable of recruiting RNase H, such as a region including 6-16 (e.g., 6, 7, 8, 9, 10, 11, 12, 13, 14, 15, or 16) DNA nucleosides. In some embodiments, the gapmer is an LNA gapmer. In some embodiments, the LNA gapmer is selected from the following uniform flank designs 4-10-4, 3-11-4, 4-11-4, 4-12-4 or 4-14-2, wherein the generic formula is 5′ flank-central block-3′ flank. In some embodiments, the LNA gapmer is selected from the following alternating flanks designs 3-1-3-10-2, 1-3-4-6-1-3-2, 1-2-1-2-2-8-4, or 3-3-1-8-2-1-2. Table 5 lists exemplary designs of each motif sequence.

In all instances the F-G-F′ design may further include regions D′ or D″, as described in the “Definitions” section under “Region D′ or D” in an ASO″. In some embodiments, the ASO of the disclosure has 1, 2, or 3 phosphodiester-linked nucleoside monomers, such as DNA monomers, at the 5′ or 3′ end of the gapmer region.

The ASO may also include at least one (e.g., at least 2, 3, 4, 5, 6, 7, 8, 9, 10, 11, 12, 13, 14, 15, or more) modified internucleoside linkages. Suitable internucleoside modifications are described in the “Definitions” section under “Modified internucleoside linkage”. In some embodiments, at least 75% (e.g., at least 76%, 80%, 85%, 90%, 95%, 96%, 97%, 98%, 99%, or more) of internucleoside linkages within the contiguous nucleotide sequence are phosphorothioate or boranophosphate internucleoside linkages. In some embodiments, all the internucleotide linkages in the ASO or a contiguous sequence thereof are phosphorothioate linkages.

For some embodiments of the disclosure, the ASO is selected from the group of ASO compounds with CMP-ID-NO: 6; 8_1; 8_3; 8_4; 10_4; 12; 14; 15; 24_1; 24_3; 26; 29_3; and 29_5.

A particular advantageous ASO in the context of the disclosure is an ASO compound selected from the group consisting of

(SEQ ID NO: 19; CMP ID NO: 26)
TTCttctttaaatcCATC;
(SEQ ID NO: 20; CMP ID NO: 8_3)
ATaAgtgttcattcTCC;
(SEQ ID NO: 21; CMP ID NO: 8_4)
ATAAgTgttcattctCC;
(SEQ ID NO: 22; CMP ID NO: 12)
CATActattcttatTTCA;
(SEQ ID NO: 23; CMP ID NO: 24_3)
TTCAtactattcttatTtCA;
(SEQ ID NO: 24; CMP ID NO: 24_1)
TTcATactattcttatTTCA;
(SEQ ID NO: 25; CMP ID NO: 29_5)
TTtCAtActattcttaTTTC;
(SEQ ID NO: 26; CMP ID NO: 8_1)
ATAagtgttcattcTCC;
(SEQ ID NO: 27; CMP ID NO: 10_4)
ATtAtgtttatcaCCAa;
(SEQ ID NO: 28; CMP ID NO: 6)
AACagtctttttacTCC;
(SEQ ID NO: 29; CMP ID NO: 29_3)
TTTCatactattcttaTTTC;
(SEQ ID NO: 30; CMP ID NO: 14)
GACAtatacactcaAATA;
and
(SEQ ID NO: 31; CMP ID NO: 15)
GTtTatcaccaaaaTTAT,

wherein non-italicized capital letters are beta-D-oxy LNA nucleosides, italicized capital letters are 2′-O-methyl nucleosides, lowercase letters are DNA nucleosides, all LNA C are 5-methylcytosine, and all internucleoside linkages are phosphorothioate internucleoside linkages.

### Methods of Manufacture

The disclosure further provides methods for manufacturing the ASOs described herein, including reacting nucleotide units under conditions and for a time suitable for forming covalently linked contiguous nucleotide polymers that are included in the ASO. The method of manufacture may use phosphoramidite chemistry (see for example Caruthers et al., Methods in Enzymology 154:287-313 (1987)). The method may further include reacting the contiguous nucleotide sequence with a conjugating moiety under conditions and for a time suitable to covalently attach the conjugate moiety to the ASO. Accordingly, the disclosure provides a method for manufacturing the ASO compounds of the disclosure or compositions containing the same, including mixing the ASO or conjugated ASO of the disclosure with a pharmaceutically acceptable diluent, solvent, carrier, salt or adjuvant.

### Pharmaceutically Acceptable Salts

The compounds according to the present disclosure may be in the form of a pharmaceutically acceptable salt. The term “pharmaceutically acceptable salt” refers to conventional acid-addition salts or base-addition salts that retain the biological effectiveness and properties of the compounds of the present disclosure and are formed from suitable non-toxic organic or inorganic acids or organic or inorganic bases. Acid-addition salts include, for example, those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium, and quaternary ammonium hydroxides, such as, for example, tetramethyl ammonium hydroxide. The chemical modification of a pharmaceutical compound into a salt is a technique well-known to pharmaceutical chemists in order to obtain improved physical and chemical stability, hygroscopicity, flowability, and solubility of compounds. Such methods are described in, e.g., Bastin, Organic Process Research & Development 4:427-35 (2000) or in Ansel, In: Pharmaceutical Dosage Forms and Drug Delivery Systems, 6th ed. (1995), pp. 196 and 1456-7. For example, the pharmaceutically acceptable salt of the compounds provided herein may be a sodium salt.

Accordingly, the disclosure provides a pharmaceutically acceptable salt of the ASO or a conjugate thereof. In some embodiments, the pharmaceutically acceptable salt is a sodium or a potassium salt. The disclosure further provides a pharmaceutical composition including the ASO or a salt or conjugate thereof.

### Pharmaceutical Compositions

The disclosure further provides pharmaceutical compositions including any of the aforementioned ASOs or salts or conjugates thereof and a pharmaceutically acceptable diluent, carrier, salt, or adjuvant. A pharmaceutically acceptable diluent may include phosphate-buffered saline (PBS), such as, e.g., sterile PBS. Pharmaceutically acceptable salts may include, but are not limited to, sodium and potassium salts. In some embodiments, the ASO is used in the pharmaceutically acceptable diluent at a concentration of 1 μM-10 mM solution (e.g., a concentration of 1 μM-8 mM, 1 μM-6 mM, 1 μM-4 mM, 1 μM-2 mM, 1 μM-500 μM, 1 μM-300 μM, 300 μM-10 mM, 500 μM-10 mM, 2 mM-10 mM, 4 mM-10 mM, 6 mM-10 mM, and 8 mM-10 mM).

Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer, Science 249:1527-1533, (1990). WO 2007/031091 provides further suitable examples of pharmaceutically acceptable diluents, carriers, and adjuvants (hereby incorporated by reference). Suitable dosages, formulations, administration routes, compositions, dosage forms, combinations with other therapeutic agents, and pro-drug formulations are also provided in WO2007/031091, which is incorporated by reference herein.

ASOs of the disclosure may be mixed with pharmaceutically acceptable, active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques or may be sterile filtered. The resulting aqueous solutions may be packaged for use as is or the aqueous solutions may be lyophilized. The lyophilized preparation may be combined with a sterile aqueous carrier prior to administration. The pH of the preparations typically will be between 3 and 11 (e.g., 3, 4, 5, 6, 7, 8, 9, 10, or 11), e.g., between 5 and 9 (e.g., 5.5, 6, 6.5, 7, 7.5, 8, 8.5, or 9), e.g., between 6 and 8 (e.g., 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8), e.g., between 7 and 8 (e.g., 7.1, 7.2, 7.3, 7.4, 7.5, 7.6, 7.7, 7.8, 7.9, or 8), e.g., 7 to 7.5 (e.g., 7.1, 7.2, 7.3, 7.4, or 7.5). The resulting compositions in solid form may be packaged in multiple single-dose units, each containing a fixed amount of the aforementioned agent(s), such as, e.g., in a sealed package of tablets or capsules.

In some embodiments, an ASO of the disclosure or a conjugate thereof is a prodrug.

### Pharmaceutical Uses

The ASOs of the disclosure may be utilized as research reagents for, e.g., diagnostics, therapeutics, and prophylaxis. In the context of research reagents, such ASOs may be used to specifically modulate the synthesis of TMEM106B protein in cells (e.g. In vitro cell cultures or tissue organoids) and experimental animals, thereby facilitating functional analysis of the target gene or facilitating a pre-clinical appraisal of its usefulness as a target for therapeutic intervention. If employing the ASO of the disclosure or a conjugate thereof in research or diagnostics, the TMEM106B target nucleic acid may be a cDNA or a synthetic nucleic acid derived from DNA or RNA.

The present disclosure provides an in vivo or in vitro method for modulating TMEM106B expression in a target cell expressing TMEM106B, said method including administering an oligonucleotide of the disclosure in an effective amount to said cell. In some embodiments, the target cell is a neuronal cell. In some embodiments, the target cell is a glial cell (e.g., microglia, astrocyte, oligodendrocyte, or Schwann cell). In some embodiments, the target cell is a mammalian cell, e.g., a human cell. The target cell may be an in vitro cell culture or tissue organoid or an in vivo cell forming part of a tissue in a mammal. The target cell may be present in the CNS, such as the brain or spinal cord. In some embodiments, the cell is a brain cell. In some embodiments, the cell is a frontal cortical cell or a frontal temporal lobe cell. In some embodiments, the target cell is a cell of the thalamus, hippocampus, striatum, retina, or spinal cord.

It will be understood that for in vitro use, such as, e.g., for evaluation of TMEM106B expression or inhibition thereof in a target cell, the cell may be isolated from the tissue or may be derived from the tissue (e.g., an established or immortalized cell line), such as CNS tissue, brain tissue, frontal cortex, frontal temporal lobe tissue, thalamus tissue, hippocampus tissue, striatum tissue, retinal tissue, or spinal cord tissue. Cells isolated from the target tissue are referred to as primary cells.

In the context of their use for diagnostic purposes, the ASOs of the disclosure may be used to detect and quantify TMEM106B expression in cell and tissues by northern blotting, in-situ hybridization, real-time quantitative polymerase chain reaction (qRT-PCR), RNA sequencing, or other techniques suitable for quantifying mRNA expression.

In the context of therapeutic applications, the ASOs of the disclosure may be administered to an animal, e.g., a human, suspected of having a disease or disorder, which can be treated by modulating the expression of TMEM106B. Exemplary conditions that may benefit from treatment with the TMEM106B-targeting compounds of the disclosure are described herein. Accordingly, the disclosure provides methods for treating or preventing a disease, including administering a therapeutically or prophylactically effective amount of an ASO of the disclosure or a pharmaceutical composition containing the same to a subject suffering from or susceptible to a disease as referred to herein. Additionally, the disclosure relates to an ASO of the disclosure or a pharmaceutical composition containing the same for use as a medicament. The ASO or the pharmaceutical composition of the disclosure can be administered in an effective amount. Further still, the disclosure provides for the use of the ASO of the disclosure for the manufacture of a medicament for the treatment of a disorder as referred to herein, or for a method of the treatment of a disorder referred to herein.

The disease or disorder may be associated with dysregulated TMEM106B expression or activity. In some embodiments, the disease or disorder may be associated with a mutation in the TMEM106B gene or a gene whose protein product is associated with or interacts with (e.g., is a binding partner of) TMEM106B. Thus, the TMEM106B target nucleic acid may be a mutated form of the TMEM106B sequence. In some embodiments, the TMEM106B target nucleic acid is a regulator of the activity or expression of TMEM106B. Thus, the disclosure further provides a use of an ASO or a pharmaceutical composition thereof for the manufacture of a medicament for the treatment of abnormal (e.g., increased) levels or activity of TMEM106B. Exemplary disorders associated with dysregulated TMEM106B expression or activity may include neurodegenerative disorders such as, e.g., frontotemporal lobar degeneration (FTLD), Parkinson's disease (or parkinsonism; PD), hypomyelinating leukodystrophies (HML), amyotrophic lateral sclerosis (ALS), Alzheimer's disease (AD), hippocampal sclerosis, corticobasal syndrome (CBS), limbic-predominant age-related TDP-43 encephalopathy (LATE), progressive supranuclear palsy (PSP), and neuronal ceroid lipofuscinosis (NCL). Given that several of these disorders are associated with dysfunction of the frontal cortex, the compounds of the disclosure may be used for the treatment of age-associated or disease-associated changes in frontal cortex.

Accordingly, ASOs described herein or pharmaceutical compositions containing the same may be suitable for use in the treatment of FTLD. In particular, the ASOs or pharmaceutical compositions of the disclosure may be advantageous in the treatment of FTLD characterized by intranuclear or cytoplasmic accumulations of ubiquitinated proteins (FTLD-U), such as, e.g., FTLD-TDP, which is characterized by the presence of ubiquitinated TAR DNA binding protein 43 (TDP-43) deposits in frontal and temporal brain regions, as well as other TDP-43 proteinopathies (e.g., ALS, Alzheimer's disease, cerebral age-related TDP-43 with sclerosis (CARTS), limbic-predominant age-related TDP-43 encephalopathy (LATE), chronic traumatic encephalopathy (CTE), and hippocampal sclerosis). In some embodiments, the pharmaceuticals compositions described herein may be useful in treating or preventing a coronavirus (e.g., COVID-19) infection.

### Antisense Oligonucleotide for In Vivo Administration

The compounds and compositions disclosed herein may be administered to a subject (e.g., a subject identified as having a disease or disorder described herein) using standard methods. For example, the compounds and compositions disclosed herein can be administered by any of a number of different routes including, e.g., systemic administration. Non-limiting examples of systemic administration include enteral (e.g., oral) or parenteral (e.g., intravenous, intra-arterial, transmucosal, intraperitoneal, epicutaneous, intramucosal (e.g., intranasal or sublingual), intramuscular, or transdermal) administration. Additional routes of administration may include intradermal, subcutaneous, and percutaneous injection. The compositions disclosed herein may also be administered using methods suitable for local delivery of ASOs or compositions containing the same. Non-limiting examples of local administration include epicutaneous (e.g., topical), intra-articular, and inhalational routes. In particular, the disclosed compositions may be locally administered to brain tissue (e.g., neural cells, such as e.g., neurons or glia) of the subject.

In particular, the compounds and compositions of the disclosure may be administered locally to brain tissue of the subject, such as brain tissue determined to be responsible for the underlying pathology in the subject. Local administration to the brain generally includes any method suitable for delivery of an ASOs or compositions containing the same to brain cells (e.g., neural cells), such that at least a portion of cells of a selected, synaptically connected cell population is contacted with the composition. ASOs may be delivered to any cells of the CNS, including neurons, glia, or both. Generally, the ASO is delivered to cells of the CNS, including, e.g., cells of the spinal cord, brainstem (medulla, pons, and midbrain), cerebellum, diencephalon (e.g., thalamus and hypothalamus), telencephalon (corpus striatum, cerebral cortex (e.g., cortical regions in the occipital, temporal, parietal, or frontal lobes), or combinations thereof, or any suitable subpopulation of cells therein. Further sites for delivery include the red nucleus, amygdala, entorhinal cortex, and neurons in ventrolateral or anterior nuclei of the thalamus.

The ASOs or compositions of the disclosure may be delivered by way of stereotactic injections or microinjections directly into the parenchyma or ventricles of the CNS. In a particular example, the ASOs of the disclosure may be delivered directly to one or more epileptic foci in the brain of the subject. For example, the subject may be administered an ASO of the disclosure or a pharmaceutical composition containing the same by means of a stereotactic injection directly into one or both hemispheres of the allocortex (e.g., hippocampus) or neocortex (e.g., frontal lobe). In a particular example, the subject is administered an ASO of the disclosure by means of a stereotactic injection directly into one or both hemispheres of the brain (e.g., frontal cortex). Alternatively, the ASOs of the disclosure may be administered by intravenous injection. In another example, the subject is administered an ASO of the disclosure intrathecally, and the ASO may be administered directly to the cisterna magna,

To deliver a ASO of the disclosure or a pharmaceutical composition containing the same specifically to a particular region and to a particular population of CNS cells, the ASO may be administered by stereotaxic microinjection. For example, subjects may have a stereotactic frame base surgically fixed in place (screwed into the skull). The brain with a stereotactic frame base (e.g., MRI compatible stereotactic frame base with fiducial markings) is imaged using high resolution MRI. The MRI images are then transferred to a computer which runs stereotactic software. A series of coronal, sagittal and axial images are used to determine the target injection site and trajectory of the cannula or injection needle used for injecting a composition of the disclosure into the brain. The software directly translates the trajectory into three-dimensional coordinates appropriate for the stereotactic frame. Holes are drilled above the entry site and the stereotactic apparatus is positioned with the injection needle implanted at the given depth. The composition (such as a composition disclosed herein) may be injected at the target sites.

Additional routes of administration may also include local application of the ASO under direct visualization, e. g., superficial cortical application, or other non-stereotactic application. The ASO may be delivered intrathecally (e.g., directly into the cisterna magna), in the ventricles (e.g., using intracerebroventricular (ICV) injection) or by intravenous injection.

In one example, the method of the disclosure includes intracerebral or intracerebroventricular administration through stereotaxic injections. However, other known delivery methods may also be adapted in accordance with the disclosure. For example, for a more widespread distribution of the composition across the CNS, it may be injected into the cerebrospinal fluid, e.g., by lumbar puncture. To direct the composition to the peripheral nervous system (PNS), it may be injected into the spinal cord, one or more peripheral ganglia, or under the skin (subcutaneously or intramuscularly) of the body part of interest. In certain situations, the composition can be administered via an intravascular approach. For example, the composition can be administered intra-arterially (carotid) in situations where the blood-brain barrier is disturbed or not disturbed. Moreover, for more global delivery, the composition can be administered during the “opening” of the blood-brain barrier achieved by infusion of hypertonic solutions including mannitol.

The most suitable route for administration in any given case will depend on the particular composition administered, the subject, the particular epilepsy being treated, pharmaceutical formulation methods, administration methods (e.g., administration time and administration route), the subject's age, body weight, sex, severity of the diseases being treated, the subject's diet, and the subject's excretion rate.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Materials and Methods

### ASO Motif Sequences and ASO Compounds

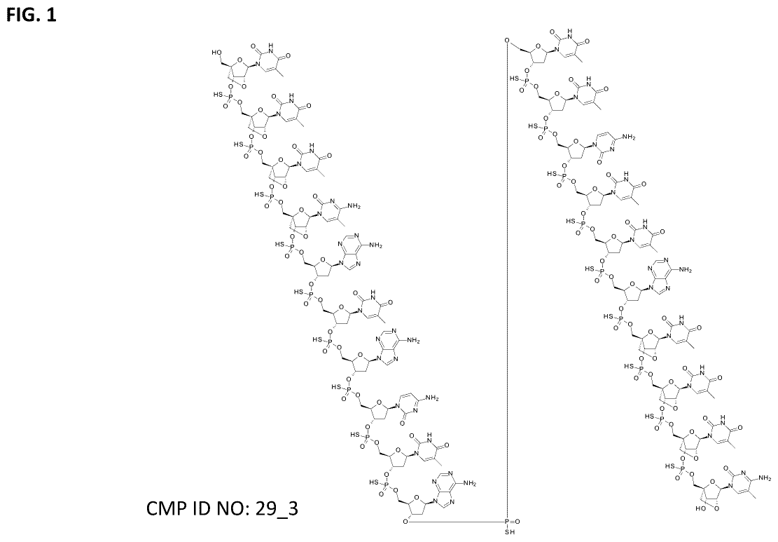

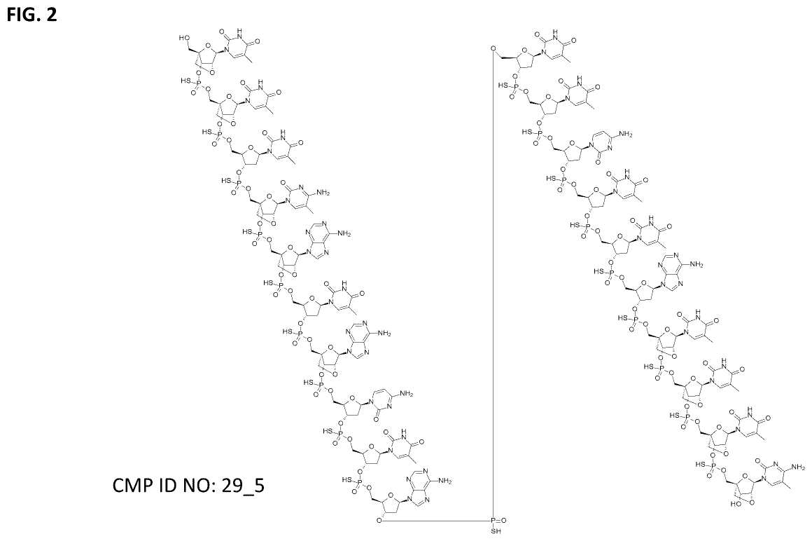

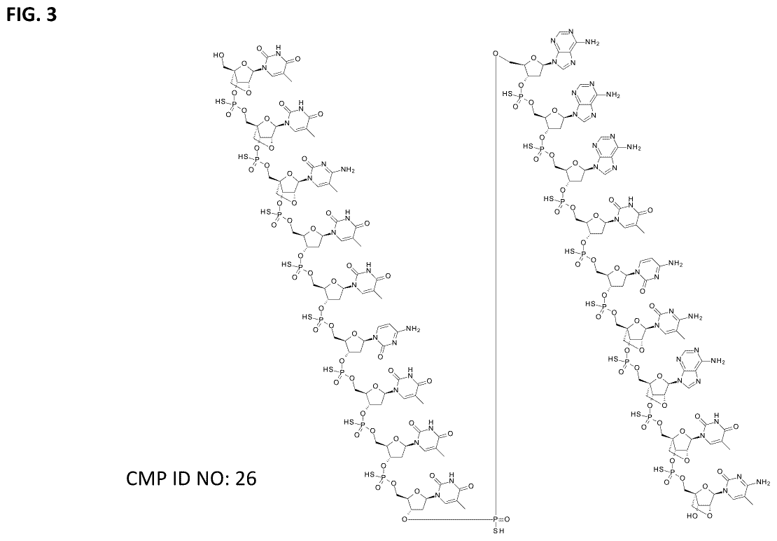

Table 5 shows a list of ASO nucleotide sequences (indicated by SEQ ID NO), the specific ASO compounds (indicated by CMP ID NO), and the HELM annotation designed by the inventors for targeting and degradation of TMEM106B mRNA. The chemical structures of these ASOs are shown in FIGS. 1-3.

TABLE 5
HELM annotation of TMEM106B-targeting antisense compounds of the disclosure

| CMP | | SEQ |
|---|---|---|
| ID NO | HELM Annotation | ID NO: |
| 6 | {[LR](A)[sP].[LR](A)[sP].[LR]([5meC])[sP].[dR](A)[sP].[dR](G) | 32 |
| | [sP].[dR](T)[sP].[dR](C)[sP].[dR](T)[sP].[dR](T)[sP].[dR](T) | |
| | [sP].[dR](T)[sP].[dR](T)[sP].[dR](A)[sP].[dR](C)[sP].[LR](T) | |
| | [sP].[LR]([5meC])[sP].[LR]([5meC])} | |
| 8_1 | {[LR](A)[sP].[LR](T)[sP].[LR](A)[sP].[dR](A)[sP].[dR](G)[sP].[dR] | 33 |
| | (T)[sP].[dR](G)[sP].[dR](T)[sP].[dR](T)[sP].[dR](C)[sP].[dR](A) | |
| | [sP].[dR](T)[sP].[dR](T)[sP].[dR](C)[sP].[LR](T)[sP].[LR]([5meC]) | |
| | [sP].[LR]([5meC])} | |
| 8_3 | {[LR](A)[sP].[LR](T)[sP].[dR](A)[sP].[LR](A)[sP].[dR](G)[sP].[dR] | 34 |
| | (T)[sP].[dR](G)[sP].[dR](T)[sP].[dR](T)[sP].[dR](C)[sP].[dR](A) | |
| | [sP].[dR](T)[sP].[dR](T)[sP].[dR](C)[sP].[LR](T)[sP].[LR]([5meC]) | |
| | [sP].[LR]([5meC])} | |
| 8_4 | {[LR](A)[sP].[LR](T)[sP].[LR](A)[sP].[LR](A)[sP].[dR](G)[sP].[mR] | 35 |
| | (U)[sP].[dR](G)[sP].[dR](T)[sP].[dR](T)[sP].[dR](C)[sP].[dR](A) | |
| | [sP].[dR](T)[sP].[dR](T)[sP].[dR](C)[sP].[dR](T)[sP].[LR]([5meC]) | |
| | [sP].[LR]([5meC])} | |
| 10_4 | {[LR](A)[sP].[LR](T)[sP].[dR](T)[sP].[LR](A)[sP].[dR](T)[sP].[dR] | 36 |
| | (G)[sP].[dR](T)[sP].[dR](T)[sP].[dR](T)[sP].[dR](A)[sP].[dR] | |
| | (T)[sP].[dR](C)[sP].[dR](A)[sP].[LR]([5meC])[sP].[LR]([5meC] | |
| | [sP].[LR](A)P.[idR](A)} | |
| 10_13 | {[LR](A)[sP].[LR](T)[sP].[dR](T)[sP].[dR](A)[sP].[dR](T)[sP].[dR] | 37 |
| | (G)[sP].[dR](T)[sP].[dR](T)[sP].[dR](T)[sP].[dR](A)[sP].[dR] | |
| | (T)[sP].[dR](C)[sP].[dR](A)[sP].[dR](C)[sP].[LR]([5meC])[sP].[LR] | |
| | (A)[sP].[LR](A)} | |
| 12 | {[LR]([5meC])[sP].[LR](A)[sP].[LR](T)[sP].[LR](A)[sP].[dR](C) | 38 |
| | [sP].[dR](T)[sP].[dR](A)[sP].[dR](T)[sP].[dR](T)[sP].[dR](C) | |
| | [sP].[dR](T)[sP].[dR](T)[sP].[dR](A)[sP].[dR](T)[sP].[LR](T) | |
| | [sP].[LR](T)[sP].[LR]([5meC])[P].[LR](A)} | |
| 13 | {[LR]([5meC])[sP].[dR](C)[sP].[LR](A)[sP].[LR](A)[sP].[LR](A) | 39 |
| | [sP].[dR](T)[sP].[dR](C)[sP].[dR](T)[sP].[dR](T)[sP].[dR](A) | |
| | [sP].[dR](T)[sP].[dR](A)[sP].[dR](A)[sP].[dR](T)[sP].[dR](A) | |
| | [sP].[LR](A)[sP].[LR]([5meC])[sP].[dR](T)[sP].[LR](A)[sP].[LR] | |
| | ([5meC])} | |
| 14 | {[LR](G)[sP].[LR](A)[sP].[LR]([5meC])[sP].[LR](A)[sP].[dR](T) | 40 |
| | [sP].[dR](A)[sP].[dR](T)[sP].[dR](A)[sP].[dR](C)[sP].[dR](A) | |
| | [sP].[dR](C)[sP].[dR](T)[sP].[dR](C)[sP].[dR](A)[sP].[LR](A) | |
| | [sP].[LR](A)[sP].[LR](T)[sP].[LR](A)} | |
| 20 | {[LR](T)[sP].[LR]([5meC])[sP].[LR](T)[sP].[LR](T)[sP].[dR](A) | 41 |
| | [sP].[dR](T)[sP].[dR](T)[sP].[dR](T)[sP].[dR](C)[sP].[dR](A) | |
| | [sP].[dR](A)[sP].[dR](A)[sP].[dR](T)[sP].[LR]([5meC])[sP].[LR] | |
| | (T)[sP].[LR]([5meC])[sP].[LR](A)} | |
| 24_1 | {[LR](T)[sP].[LR](T)[sP].[dR](C)[sP].[LR](A)[sP].[LR](T)[sP].[LR] | 42 |
| | (A)[sP].[dR](C)[sP].[dR](T)[sP].[dR](A)[sP].[dR](T)[sP].[dR] | |
| | (T)[sP].[dR](C)[sP].[dR](T)[sP].[dR](T)[sP].[dR](A)[sP].[dR](T) | |
| | [sP].[LR](T)[sP].[LR](T)[sP].[LR]([5meC])[sP].[LR](A)} | |
| 24_3 | {[LR](T)[sP].[LR](T)[sP].[LR]([5meC])[sP].[LR](A)[sP].[dR](T) | 43 |
| | [sP].[dR](A)[sP].[dR](C)[sP].[dR](T)[sP].[dR](A)[sP].[dR](T) | |
| | [sP].[dR](T)[sP].[dR](C)[sP].[dR](T)[sP].[dR](T)[sP].[dR](A) | |
| | [sP].[dR](T)[sP].[LR](T)[sP].[dR](T)[sP].[LR]([5meC])[sP].[LR](A)} | |
| 25_1 | {[LR](T)[sP].[LR](T)[sP].[LR]([5meC])[sP].[LR](T)[sP].[dR](T) | 44 |
| | [sP].[dR](A)[sP].[dR](T)[sP].[dR](T)[sP].[dR](T)[sP].[dR](C) | |
| | [sP].[dR](A)[sP].[dR](A)[sP].[dR](A)[sP].[dR](T)[sP].[LR] | |
| | ([5meC])[sP].[LR](T)[sP].[LR]([5meC])[sP].[LR](A)} | |
| 26 | {[LR](T)[sP].[LR](T)[sP].[LR]([5meC])[sP].[dR](T)[sP].[dR](T) | 45 |
| | [sP].[dR](C)[sP].[dR](T)[sP].[dR](T)[sP].[dR](T)[sP].[dR](A) | |
| | [sP].[dR](A)[sP].[dR](A)[sP].[dR](T)[sP].[dR](C)[sP].[LR]([5meC]) | |
| | [sP].[LR](A)[sP].[LR](T)[sP].[LR]([5meC])} | |
| 29_3 | {[LR](T)[sP].[LR](T)[sP].[LR](T)[sP].[LR]([5meC])[sP].[dR](A) | 46 |
| | [sP].[dR](T)[sP].[dR](A)[sP].[dR](C)[sP].[dR](T)[sP].[dR](A) | |
| | [sP].[dR](T)[sP].[dR](T)[sP].[dR](C)[sP].[dR](T)[sP].[dR](T) | |
| | [sP].[dR](A)[sP].[LR](T)[sP].[LR](T)[sP].[LR](T)[sP].[LR]([5meC])} | |
| 29_5 | {[LR](T)[sP].[LR](T)[sP].[dR](T)[sP].[LR]([5meC])[sP].[LR](A) | 47 |
| | [sP].[dR](T)[sP].[LR](A)[sP].[dR](C)[sP].[dR](T)[sP].[dR](A) | |
| | [sP].[dR](T)[sP].[dR](T)[sP].[dR](C)[sP].[dR](T)[sP].[dR](T) | |
| | [sP].[dR](A)[sP].[LR](T)[sP].[LR](T)[sP].[LR](T)[sP].[LR]([5meC])} | |
| 15 | {[LR](G)[sP].[LR](T)[sP].[dR](T)[sP].[LR](T)[sP].[dR](A)[sP].[dR] | 48 |
| | (T)[sP].[dR](C)[sP].[dR](A)[sP].[dR](C)[sP].[dR](C)[sP].[dR] | |
| | (A)[sP].[dR](A)[sP].[dR](A)[sP].[dR](A)[sP].[LR](T)[sP].[LR] | |
| | (T)[sP].[LR](A)[sP].[LR](T)} | |
| 31 | {[LR](A)[sP].[LR](T)[sP].[LR](G)[sP].[LR](T)[sP].[dR](T)[sP].[dR] | 49 |
| | (T)[sP].[dR](A)[sP].[dR](T)[sP].[dR](C)[sP].[dR](A)[sP].[dR] | |
| | (C)[sP].[dR](C)[sP].[dR](A)[sP].[dR](A)[sP].[LR](A)[sP].[LR] | |
| | (A)[sP].[LR](T)[sP].[LR](T)} | |
| 46 | {[LR](T)[sP].[LR](G)[sP].[dR](T)[sP].[dR](T)[sP].[dR](T)[sP].[dR] | 50 |
| | (A)[sP].[dR](C)[sP].[dR](A)[sP].[dR](T)[sP].[dR](A)[sP].[dR] | |
| | (T)[sP].[dR](C)[sP].[dR](T)[sP].[LR]([5meC])[sP].[LR](A)[sP].[LR] | |
| | ([5meC])[sP].[LR]([5meC])} | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methylcytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[dR]([PPG]) is a DNA 7-deaza-8-aza-guanine nucleoside,
[idR](G) is an inverted DNA guanine nucleoside,
[idR](T) is an inverted DNA thymine nucleoside,
[idR](A) is an inverted DNA adenine nucleoside,
[idR](C) is an inverted DNA cytosine nucleoside
[mR](G) is a 2′-O-methyl RNA guanine nucleoside,
[mR](U) is a 2′-O-methyl RNA uracil nucleoside,
[mR](A) is a 2′-O-methyl RNA adenine nucleoside,
[mR](C) is a 2′-O-methyl RNA DNA cytosine nucleoside,
[sP] is a phosphorothioate internucleoside linkage,
P is a phosphodiester internucleoside linkage.

Antisense oligonucleotide sequences represent the contiguous sequence of nucleobases present in the ASO. Designs refer to the gapmer design, e.g., F-G-F′. In classic gapmer design, e.g., 3-10-3 (5′ flank nucleotides-central block nucleotides-3′ flank nucleotides), all of the nucleotides in the flanks (F and F′) are constituted of the same 2′-sugar-modified nucleoside (e.g., LNA, cET, or MOE), and a stretch of DNA in the central block forming the gap (G). In gapmers with alternating flank designs, the flanks of an ASO are annotated as a series of integers, representing a number of 2′ sugar-modified nucleosides (M) followed by a number of DNA nucleosides (D). For example, a flank with a 2-2-1 motif represents 5′ [M]₂-[D]₂-[M]₃′ and a 1-1-1-1-1 motif represents 5′ [M]-[D]-[M]-[D]-[M] 3′. Both flanks have a 2′ sugar-modified nucleoside at the 5′ and 3′ terminal. The gap region (G), which includes DNA nucleosides (typically between 6 and 16), is located between the flanks.

### Antisense Oligonucleotide Synthesis

ASO synthesis is generally known in the art. Described below is an exemplary protocol which may be employed to produce ASOs having desired sequences and modifications. ASOs are synthesized on uridine universal supports using the phosphoramidite approach on an Oligomaker 48 at 1 μmol scale. At the end of the synthesis, the ASOs are cleaved from the solid support by treating the ASOs with aqueous ammonia for 5-16 hours at 60° C. The ASOs are purified by reverse phase HPLC (RP-HPLC) or by solid phase extractions and characterized by ultra-performance liquid chromatography (UPLC), and the molecular mass is further confirmed by electrospray ionization mass spectrometry (ESI-MS).

### Elongation of the Antisense Oligonucleotide

The coupling of β-cyanoethyl-phosphoramidites (e.g., DNA-A(Bz), DNA-G(ibu), DNA-C(Bz), DNA-T, LNA-5-methyl-C(Bz), LNA-A(Bz), LNA-G(dmf), or LNA-T) was performed by using a solution of 0.1 M of 5′-O-DMT-protected amidite in acetonitrile and DCI (4,5-dicyanoimidazole) in acetonitrile (0.25 M) as an activator For the final cycle, a phosphoramidite with desired modifications was used (e.g. a C6 linker for attaching a conjugate group or a conjugate group as such). Thiolation for introduction of phosphorthioate linkages was carried out using xanthane hydride (0.01 M in acetonitrile/pyridine at a 9:1 ratio). Phosphodiester linkages were introduced using 0.02 M iodine in THF/Pyridine/water at a 7:2:1 molar ratio.

For post-solid phase synthesis conjugation, a commercially available C6 amino linker phosphoramidite was used in the last cycle of the solid phase synthesis. After deprotection and cleavage from the solid support, the amino-linked, deprotected ASO was isolated. The conjugate moieties were introduced via activation of the functional group using standard synthesis methods.

### Purification by Reverse Phase High-Performance Liquid Chromatography

The crude compounds were purified by preparative RP-HPLC on a Phenomenex Jupiter 10 μm C18 150×10 mm column. 0.1 M ammonium acetate, pH 8, and acetonitrile was used as a buffer at a flow rate of 5 mL/min. The collected fractions were lyophilized to produce a purified compound, which typically appeared as a white solid.

### Abbreviations

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl

### Synthesis of Inverted Oligonucleotides

The term “inverted nucleoside” or “reverse nucleoside” (DNA X^{INV}) refers to a nucleoside which includes dimethoxytrityl (DMT) and phosphoramidite groups reversed from the typical orientation; the DMT-group is attached to the 3′-OH, and the phosphoramidite attached to the 5′-OH of the ribose moiety.

The DNA X^{INV} (X=A, T, C, or G) was incorporated as a phosphoramidite into an oligonucleotide during phosphoramidite oligonucleotide synthesis (see Scheme 1, below) This reverse configuration allowed oligonucleotide synthesis in the 5′ to 3′ direction (instead of the standard 3′ to 5′ direction) and afforded to 5′-5′ or 3′-3′ nuclease-resistant linkages instead of the natural 3′-5′ linkage (see Scheme 2, below). See also Ortigao et al., Antisense Res. Dev., 2:129-146 (1992). Exemplary inverted nucleotides are shown in Scheme 2.

To improve the safety profile of the designed ASO compounds, the phosphorothioate backbone was modified with inverted phosphodiester linkages to reduce phosphorothioate content and, resultantly, the chiral complexity of the ASO, while also retaining resistance to exonucleases.

As an example, for the synthesis of ASO of CMP ID NO: 10_4 containing a 3′-3′ phosphodiester linkage at the 3′ end, the appropriate Bz-dA-5′-CE phosphoramidite was first attached to a universal support during the first cycle of the phosphoramidite oligonucleotide synthesis, followed by the addition of the second monomer, namely Bz-LNA A-3′-CE phosphoramidite, during the second cycle of the phosphoramidite oligonucleotide synthesis in the standard 3′-5′ direction to make the corresponding terminal 3′-3′ linkage. Having a single inverted base at the 3′ end with a 3′-3′ linkage imparts oligonucleotide exonuclease resistance and prevents extension by polymerases, as there is no free 3′ hydroxyl group to initiate synthesis.

### Scheme 1: DNA 5′ Phosphoramidites for the Synthesis of a Reverse 3′-3′ or 5′-5′ Internucleoside Linkage

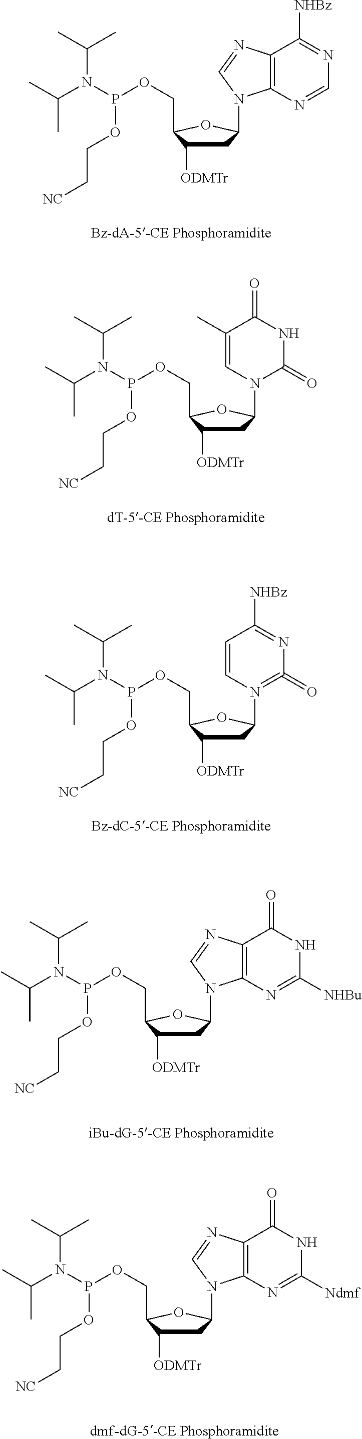

### Scheme 2: Design of Phosphorothioate Internucleoside Linkage and Inverted Phosphodiester Internucleoside Linkage

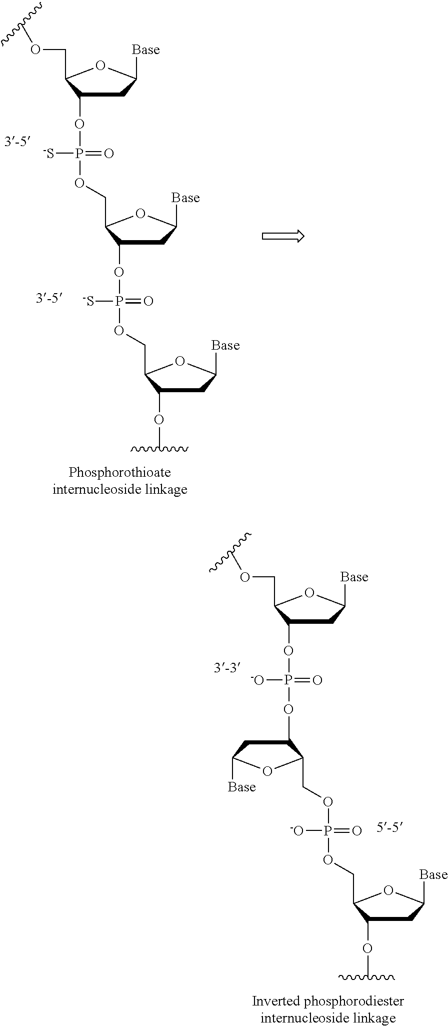

### Example 1: Antisense Oligonucleotides Reduce TMEM106B mRNA Expression in Human SK-N-BE(2) Neuroblastoma Cells

As an initial screen, over 1000 designed TMEM106B-targeting ASOs were tested for their ability to reduce TMEM106B mRNA expression in human SK-N-BE(2) neuroblastoma cells (ECACC; Catalog No. 95011815). SK-N-BE(2) cells were grown in cell culturing media of Minimum Essential Media (MEM) (Sigma, Cat. No. M2279) mixed with Nutrient Mixture F-12 Ham (Sigma, Cat. No. N4888) in a 1:1 (v/v) ratio, supplemented with 1 mM glutamine (Sigma, Cat. No. G7513), and 0.025 mg/mL gentamicin (Sigma, Cat. No. G1397), 15% fetal bovine serum (FBS) (Sigma, Cat. No. F7524), and 10% FBS for all subsequent weeks. Cells were trypsinized every five days by washing with PBS (Sigma, Cat. No 14190-094), followed by addition of 0.25% Trypsin-EDTA solution (Sigma, Cat. No. T3924), and 2-3 minutes of incubation at 37° C. The cells were then subjected to trituration before cell seeding. Cells were maintained in culture for up to 15 passages.

As a next step, 20,000 cells per well were seeded in 96-well plates (Nunc, Cat. No. 167008) in 190 μL growth media. ASOs dissolved in PBS were added approximately 24 hours after the cells were seeded to reach final concentrations. The cells were incubated for three days without any media change. After incubation, cells were harvested by removal of media followed by addition of 125 μL RLT Lysis buffer (Qiagen, Cat. No. 79216) and 125 μL of 70% ethanol. The RNA was purified according to the manufacture's instruction (Qiagen RNease 96 kit) and eluted into a final volume of 50 μL of water, followed by a further 10-fold dilution in water prior to qPCR.

The RNA was heat-shocked for 40 seconds at 90° C. to denature the RNA:LNA duplexes and moved directly to ice and spun down before use. For a one-step qPCR reaction, the qPCR-mix, qScript™ XLE 1-step RT-qPCR TOUGHMIX® Low ROX, (QauntaBio, Cat. No. 95134-500) was mixed with two IDT probes having a concentration of 1× to generate the Mastermix. Taqman probes were acquired from IDT:TMEM:Hs.PT.58.3604573 (primer-probe ratio 2, FAM); POLR2A:Hs.PT.39.a19639531 (primer-probe ratio 2, HEX/VIC). Next, 6 μL of Mastermix and 4 μL RNA with a concentration of 1-2 ng/μL were then mixed in a qPCR plate (MICROAMP® optical 384 well, Cat. No. 4309849). After sealing, the plate was given a spin at 1000 g for 1 minute at room temperature and transferred to a Viia™ 7 system (Applied Biosystems, Thermo). The following PCR conditions were used: 50° C. for 15 minutes, 95° C. for 3 minutes, 40 cycles of: 95° C. for 5 seconds, followed by a temperature decrease of 1.6° C./sec, followed by 60° C. for 45 sec. The data was analyzed using the QuantStudio™ Real_time PCR Software.

The qPCR data was captured and quality control on the raw data was performed using Quantstudio 7 software. The data was then imported into E-Workbook, in which a BioBook template was used to capture and analyze the data. The data were analyzed using the following steps:
1. RNA quantity was calculated by the delta Ct method (Quantity=2\hat{ }(−Ct)*1,000,000,000).
2. RNA quantity was normalized to the calculated quantity for the housekeeping gene assay run in the same well. Relative Target Quantity=QUANTITY_target/QUANTITY_housekeeping.
3. The degree of RNA knockdown was calculated for each well by division with the mean of all PBS-treated wells on the same plate. Normalized Target Quantity=(Relative Target Quantity/[mean] Relative Target Quantity]_PBS_wells)*100
4. The final data were shown as a percentage of RNA relative to untreated (PBS) wells.
5. For concentration-response experiments, a curve was fitted from the RNA knockdown values (step 3-4) for each compound using either 8 or 10 concentrations, depending on the dilution model. Curves are fitted using a 4 Parameter Sigmoidal Dose-Response Model in Biobook.

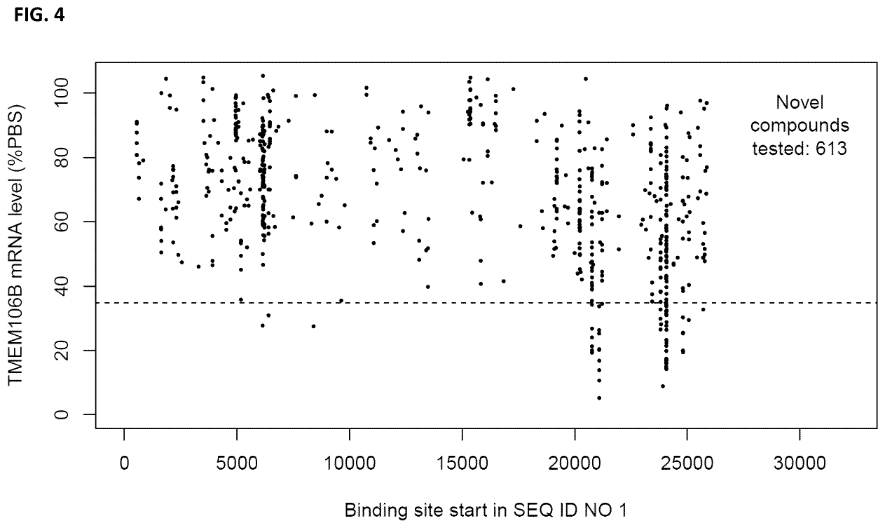

In one set of experiments, 613 novel human TMEM106B targeting ASO compounds were tested at 2.5 μM concentration for three days. The experiment also included 22 published human TMEM106B-targeting compounds, including CMP ID NO: 25_1 and CMP ID NO: 20, which have been described previously in U.S. Patent Application Publication No. US 2021/0054383, which is incorporated by reference herein in its entirety. The knockdown of human TMEM106B induced by the designed compounds was measured, quantified, and the specific ASO sequences were mapped onto their region of hybridization within the human TMEM106B transcript of SEQ ID NO: 1 (FIG. 4).

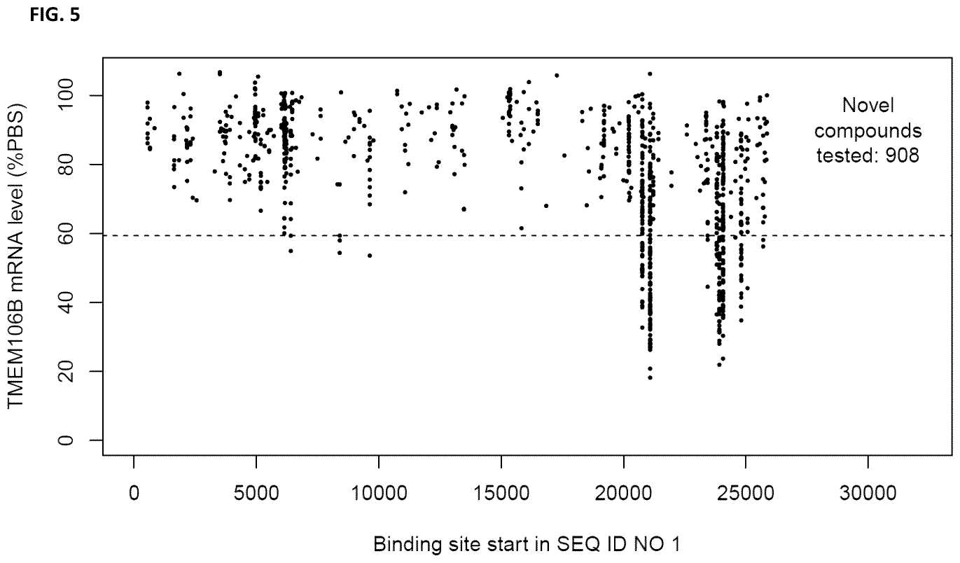

In another set of experiments, 908 novel human TMEM106B ASO compounds were tested at 0.5 μM concentration for three days. The experiment also included 22 published human TMEM106B-targeting compounds, including CMP ID NO: 25_1 and CMP ID NO: 20, which have been described previously in U.S. Patent Application Publication No. US 2021/0054383, which is incorporated by reference herein in its entirety. The knockdown of human TMEM106B induced by the novel compounds was quantified and the specific ASO sequences were mapped onto their region of hybridization within the human TMEM106B transcript of SEQ ID NO: 1 (FIG. 5). Compounds CMP ID NOs: 8_1, 8_3, 24_1, 6, 10_4, 29_5, 15, 24_3, 12, 29_3, 26, and 8_4, along with others exhibited greater knockdown relative to CMP ID NO: 31, along with other ASOs showed superior inhibition of TMEM106B expression relative to CMP ID NO: 31.

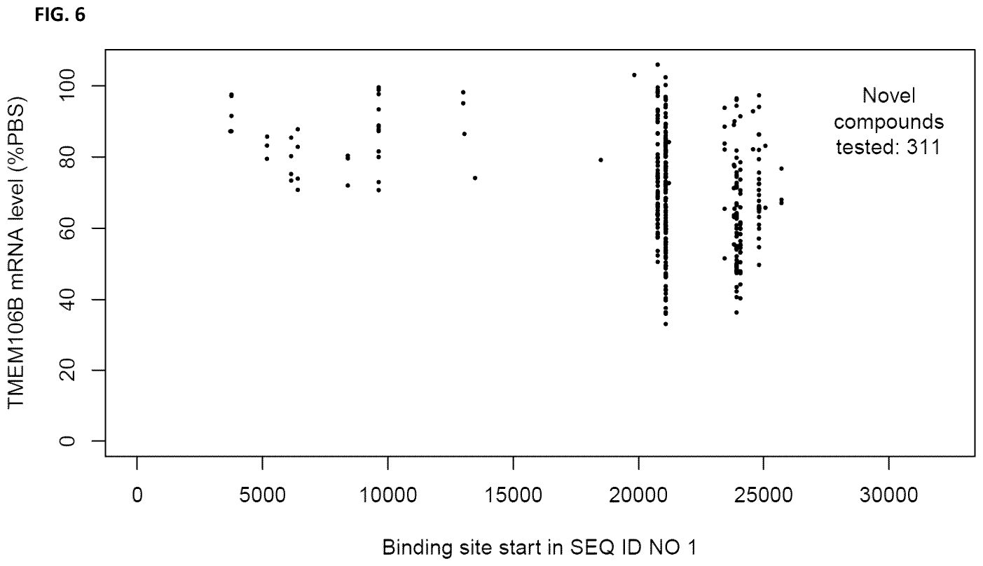

In yet another experiment, 311 novel human TMEM106B-targeting compounds were tested at 0.25 μM concentration. The experiment also included two published human TMEM106B-targeting compounds, including CMP ID NO: 25_1 and CMP ID NO: 46, which are previously described in U.S. Patent Application Publication No. US 2021/0054383, which is incorporated by reference herein in its entirety. The knockdown of human TMEM106B induced by the novel compounds was quantified and the specific ASO sequences were mapped onto their region of hybridization within the human TMEM106B transcript of SEQ ID NO: 1 (FIG. 6). Compounds CMP ID NO: 8_1, CMP ID NO: 8_3, CMP ID NO: 6, CMP ID NO: 8_4, and CMP ID NO: 29_5, along with other ASOs showed superior inhibition of TMEM106B expression relative to CMP ID NO: 25_1. Results of the above three experiments are shown in detail in Table 6, below.

TABLE 6
TMEM106B knockdown in SK-N-BE(2) glioblastoma cells treated
with antisense oligonucleotide compounds of the disclosure

| | TMEM106B mRNA | TMEM106B mRNA | TMEM106B mRNA |
|---|---|---|---|
| | (% of PBS control) | (% of PBS control) | (% of PBS control) |
| CMP ID NO | ASO: 0.25 μM | ASO: 0.5 μM | ASO: 2.5 μM |
| 31 | — | 59.33 | 34.79 |
| 29_5 | 47.35 | 39.04 | — |
| 10_13 | 94.67 | 92.15 | — |
| 20 | — | 35.59 | 11.78 |
| 25_1 | 50.21 (52.61, 44.56, | 38.34 (33.07, 37.92, | 12.82 (11.68, 13.48, |
| | 51.35, 52.32) | 42.41, 37.13, 42.74, | 11.96, 12.83, 16.67, |
| | | 41.07, 37.42, 39.58, | 12.32, 11.36, 11.95, |
| | | 41.87, 34.31, 32.39, | 13.14) |
| | | 39.53, 39.00) | |
| 15 | 66.55 | 50.14 (49.55, 50.73) | 20.16 |
| 24_3 | — | 38.4 | 17.41 |
| 8_3 | 49.21 | 33.3 | — |
| 8_4 | 49.61 | 34.55 | — |
| 14 | — | 66.59 | 35.77 |
| 8_1 | 40.65 | 28.87 | — |
| 6 | 49.7 | 34.8 | — |
| 10_4 | 54.55 | 29.09 | — |
| 24_1 | 40.3 | 23.71 | — |
| 12 | — | 45.9 | 16.88 |
| 29_3 | — | 36.27 | 14.18 |
| 26 | 66.18 | 49.58 (46.25, 52.90) | 19.98 |
| 13 | 101.61 (102.14, 99.78, | 95.61 (96.93, 101.59, | 69.68 (90.95, 97.69, |
| | 95.00, 109.51) | 86.22, 103.93, 93.96, | 99.62, 98.45) |
| | | 97.52, 91.48, 93.21) | |

### Example 2: In Vitro Efficacy of Antisense Oligonucleotide Targeting Human TMEM106B

### IC50 Value Determination for TMEM106B-Targeting Antisense Oligonucleotides

Human TMEM106B-targeting ASO compounds were initially screened in a human neuroblastoma cell line, SK-N-BE(2), as is described in Example 1. In order to have better translation to human patients, the efficacy of candidate ASO compounds against human TMEM106B was tested in human neurons derived from induced pluripotent stem cells (iPSCs) and co-cultured with human primary astrocytes.

Human iPSC-derived neurons were harvested with RLT buffer. RNA extractions were completed with Qiagen RNeasy 96 kit. RNA sample quality was measured, and the sample was subsequently subjected to RNAseq. DNA libraries were prepared from the samples and sequenced in batches of up to 48 samples at a time. ASOs were diluted from stock in an intermediate 96-well plate in a serial dilution with culture-based media buffer. Serial dilutions were designed to obtain concentrations of 10× of the final specified concentration to reduce culture media dilution after addition to neurons. On day 7, cells were harvested for Taqman assay with Cells to Ct reagent (ThermoFisher) according to manufacturer's instructions. The relative expression levels obtained using the Taqman assay were normalized to control wells, and the data was imported into Graph Pad Prism software for IC50 analysis, which was completed with a software curve fit function.

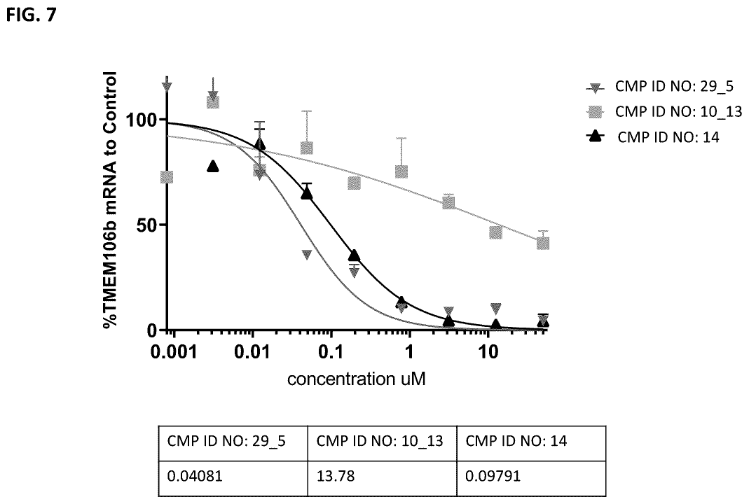

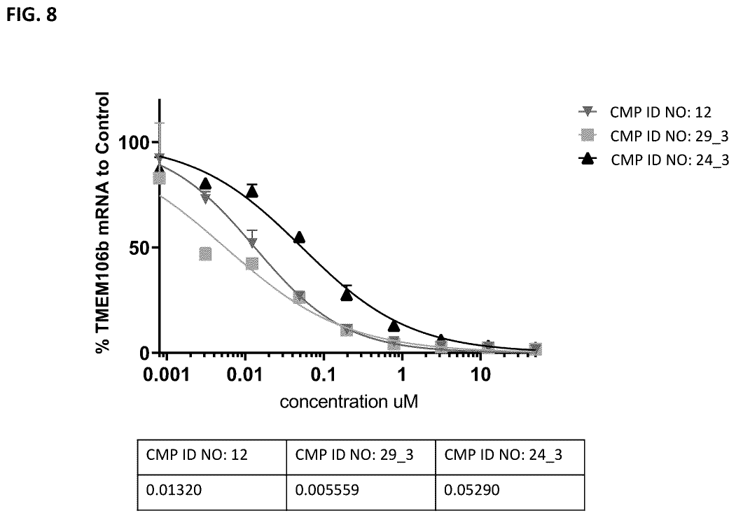

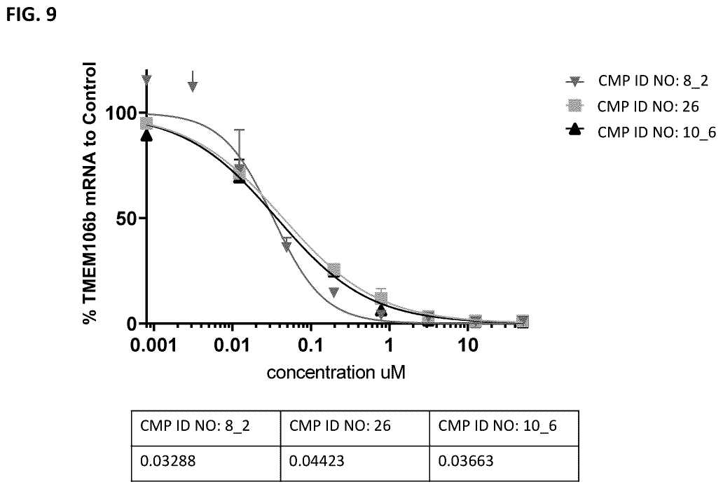

Of all of the tested compounds, 27 ASOs were selected for characterization with IC50 curves after seven days of treatment. Two prior-published ASOs, namely CMP ID NO: 31 and 25_1, were included as performance benchmarks for the newly designed ASO compounds of the disclosure. The newly designed ASOs were found to have an IC50 concentration in the range of 4.6 nM to 175 nM (Table 7 and FIGS. 7-9), which indicate high potency of the tested compounds. Furthermore, near-complete suppression of human neuronal TMEM106B mRNA was observed, indicating a promising therapeutic potential. From this screen, ASOs were identified that have improved potency as compared to CMP ID NO: 25_1, including CMP ID NOs: 8_3, 8_4, 8_1, 29_3, and 12, along with other ASOs. All 27 ASO compounds had an improved IC50 compared to CMP ID NO: 31.

### Off-Target Analysis

The potential off-target activity of the candidate ASO compounds was evaluated in a selected set of 18 ASOs from the IC50 experiments. The results of these studies are summarized in Table 7, below. Cells were treated the ASO at 5 μM, 0.5 μM, and 0.05 μM and performed bulk RNAseq. The ASO concentrations were chosen at 1-5×, 10×, and ˜100-1000× of the IC50 to stress test the ASO compounds for off-target effects, which only have 1-2 base-pair mismatched, inserted, or deleted between the ASO and an off-target gene and thus might have weak activities and may have needed significant amount of ASO concentration to be observed. At 5 μM, potential ASO off-target effects were detected. This concentration was 100×-1000× of the IC50 of the ASO tested; such a concentration would not normally occur in vivo. ASO-treated samples were compared to a non-targeting control ASO (CMP ID NO: 13) in order to identify changes in expression of transcripts other than the intended TMEM106B target. Volcano plots were produced and analyzed to determine potential off-target effects based on bioinformatics prediction of 1 or 2 base pair mismatches. The ASOs in this group were predicted to have a potential 56 to 1069 protein-coding off-target genes identified via bioinformatics searches. However, it was found that some of the ASOs tested (CMP ID NOs: 10_4, 29_3, 24_3, and 29_5, along with other ASOs) had less than 5 off-target genes showing more than 50% mRNA reduction at this non-physiological concentration. There were no off-target gene identified at 0.5 μM or 0.05 μM ASO concentrations, which are closer physiological concentrations of ASOs administered to subjects. These findings indicate that these some ASOs may have a therapeutically advantageous safety profile. Some of the ASOs tested showed off-target binding to 6-15 observed genes at 5 μM. These off-target effects were significantly reduced as ASO concentration was reduced to 0.5 μM and 0.5 μM. Significantly, CMP ID NO: 24_3 had no detectable off-target effects in human neurons, even at the non-physiological ASO concentration of 5 μM. In contrast, other ASOs, including CMP ID NO: 8_1, were found to bind to more than 27 off-target genes. The number of off-target genes targeted by these ASOs were reduced as the ASO concentration was decreased. Combined, nearly all of the identified off-target genes were either not detected or reduced when the ASO was at the 0.05 μM concentration, which was close to the IC50 concentration. The above findings are summarized for different ASO compounds in Table 7. These findings indicate a carefully selected ASO concentration may confer selective inhibition of TMEM106B expression and reduce off-target effects.

TABLE 7
Functional characterization of anti-TMEM106B ASO compounds

| | | In vivo | | | Mm1 | Mm2 | Observed | Off-target | Off-target | Off-target |
|---|---|---|---|---|---|---|---|---|---|---|
| CMP | IC50 | RNA | Mm1 | Mm2 | protein | protein | off-target | genes at | at | at |
| ID NO | (nM) | remaining | genes | genes | coding | coding | at 5 μM | 5 μM | 0.5 μM | 0.05 μM |
| 14 | 97.91 | 60% | 4 | 185 | 4 | 143 | — | — | — | — |
| 26 | 44.23 | 30% | 3 | 85 | 2 | 56 | 2 | LINC01322, | 1 | 0 |
| | | | | | | | | NSUN2 | | |
| 15 | 159.3 | 50% | 18 | 1047 | 10 | 692 | — | — | — | 0 |
| 10_4 | 68.2 | 50% | 4 | 162 | 4 | 121 | 1 | REV1 | — | 0 |
| 8_3 | 8.41 | — | 21 | 606 | 13 | 415 | — | — | — | — |
| 8_4 | 4.624 | — | 21 | 606 | 13 | 415 | — | — | — | 0 |
| 8_1 | 12.37 | 30% | 21 | 606 | 13 | 415 | 27 | LRPPRC, NSF, | 18 | 16 |
| | | | | | | | | GPHN, FGGY, | | |
| | | | | | | | | RGS7, | | |
| | | | | | | | | NSMCE2, | | |
| | | | | | | | | LOC100506207, | | |
| | | | | | | | | AL365184.1, | | |
| | | | | | | | | PHKB, MEG3, | | |
| | | | | | | | | S1PR1 | | |
| 29_3 | 5.559 | 25% | 17 | 494 | 8 | 313 | 4 | BBS4, | — | 0 |
| | | | | | | | | HSC17B4, | | |
| | | | | | | | | HSD17B4, | | |
| | | | | | | | | TUBE1 | | |
| 12 | 13.2 | — | 22 | 1140 | 11 | 731 | — | — | 1 | 0 |
| 24_3 | 52.9 | — | 42 | 1643 | 31 | 1069 | 0 | — | 0 | 0 |
| 25_1 | 30.05 | 25% | 37 | 1284 | 28 | 856 | — | — | — | — |
| 24_1 | 36.57 | 30% | 42 | 1643 | 31 | 1069 | — | — | — | — |
| 29_5 | 40.81 | — | 17 | 494 | 8 | 313 | 0 | — | 0 | — |
| 6 | 6.966 | 60% | 6 | 415 | 5 | 289 | — | — | — | — |
| 31 | 277.3 | 50% | — | — | — | — | — | — | — | — |
| 10_13 | 13780 | 95% | 4 | 162 | 4 | 121 | — | — | — | — |

Table 7 Description:
Table 7 is a summary table of the ASOs selected from the screen for further characterization. Previously identified ASOs of CMP ID NO: 25_1 and 31 were included for comparison. IC50 values are listed. Mm1 genes: 1 base pair mismatched genes. Mm2 genes: 2 base pair mismatched genes. Mm1 protein-coding: 1 base pair mismatched genes that are protein-coding. Mm2 protein-coding: 2 base pair mismatched genes that are protein-coding. Observed off-target @ 5 μM: off-target genes with more than 50% mRNA reduction in human iPSC neurons at 5 μM ASO concentration. Off-target genes @ 5 μM: the names of the first 10 off-target genes. Observed off-target @ 0.5 μM: off-target genes with more than 50% mRNA reduction found in human iPSC neurons at 0.5 μM ASO concentration. Observed off-target @ 0.05 μM: off-target genes with more than 50% mRNA reduction found in human iPSC neurons at 0.05 μM ASO concentration.

### Example 3: Functional Assays of Lysosomal Proteolysis in Human Retinal Pigment Epithelial Cells

To determine if TMEM106B-targeting ASOs of the disclosure impact lysosomal function, functional assays were performed in human retinal pigment epithelial (RPE) cells in vitro. Human (RPE) cells were selected as a model for this experiment due to their flat cell morphology, which facilitates visualization of lysosomes using known imaging techniques. Human RPE cells were purchased from ATCC and cultured according to the manufacturer's instructions.

The cells were treated with a self-quenched BSA that can be readily internalized and trafficked to lysosomes. This was performed by plating human RPE cells at 1000 cells/well in 1% Geltrex-coated 384-well plates. Cells were treated with ASO for five days in 0.5% serum media to prevent overgrowth. 200 nM bafilomycin was used as a positive control. Self-quenched BODIPY-FL conjugates of BSA were purchased from BioVision (7932-10) and experiments were performed according to manufacturer's suggested protocol. SA was added at a final concentration of 20 μg/mL and incubated overnight. Cells were fixed with 4% PFA and 4% sucrose at room temperature for 20 minutes using Bravo automation. Fixed cells were then washed two times with PBS using Biotek 406 microplate washer (Beckman Coulter), followed by permeabilization and blocking by incubation with a solution containing 1×PBS, 0.1% Triton X-100, 2% donkey serum, and 1% BSA at room temperature for 30 minutes. The blocking solution was removed, and cells were incubated with primary antibodies in blocking solution overnight at 4° C. Cell were blocked and then stained for Hoechst (Sigma 1:10,000) and LAMP1 (BD 1:500). After washing six times with PBS on Biotek 406 cell plate washer (Beckman Coulter), cells were incubated with fluorophore-conjugated secondary antibodies (Jackson Immuno Research Laboratories, Inc) for one hour at room temperature in the dark to avoid photobleaching. Cells were then washed six more times with PBS before imaging. Fluorescent images were captured using an InCell 6000 confocal microscope (GE Healthcare Life Sciences). Specifically, 16 fields were imaged per well at 40× magnification. Each treatment condition was represented by four wells. The BSA signal was segmented and total intensity was summed from all 16 fields as the total calculation per well. The image analysis of the BSA signal was performed with InCell 6000 analysis software. Quadruplicates were averaged and plotted with error bars representing the standard error of the mean (SEM).

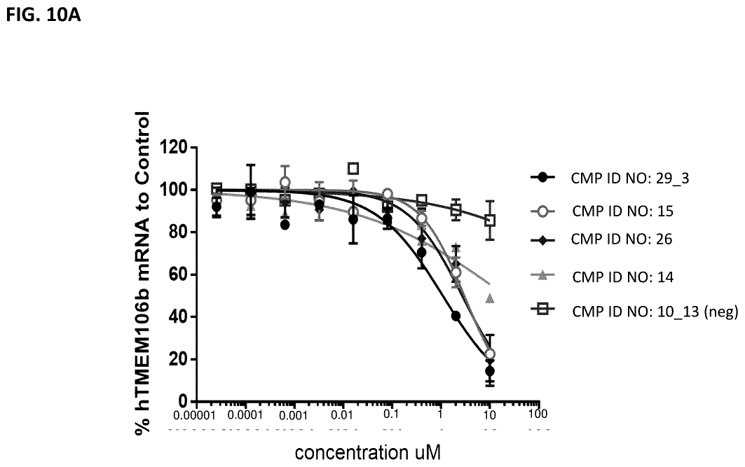

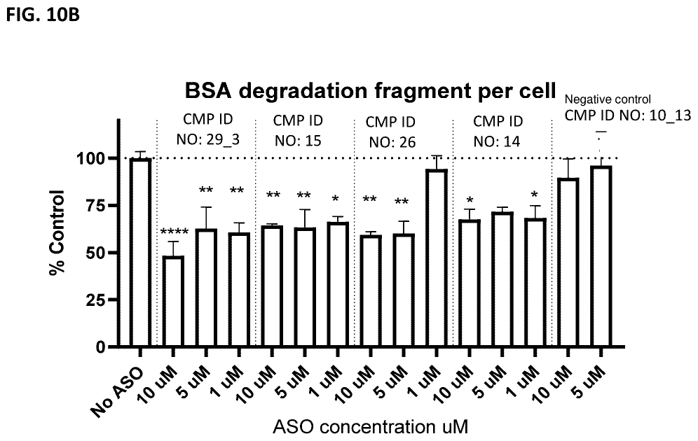

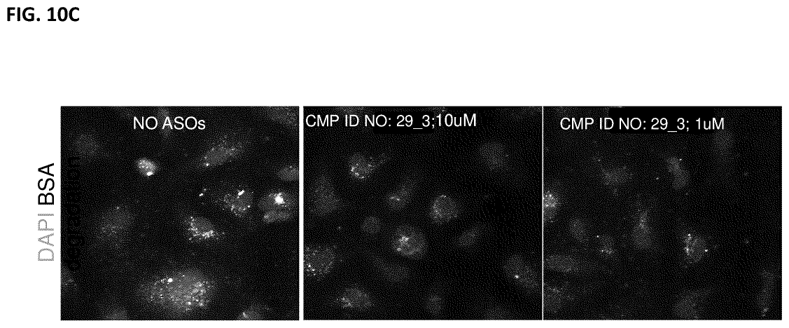

Upon degradation, the degraded and separated BSA fragments fluoresced and allowed the visualization and quantification of lysosome proteolytic activity (FIG. 10C, white dots). Using this assay, it was observed that ASOs of CMP ID NOs: 29_3, 15, 26, and 14 effectively reduced BSA degradation activity by about 50%. These findings indicate that lysosome function was reduced with the ASO-mediated degradation of TMEM106B (FIGS. 10B and 10C).

A select panel of TMEM106B-targeting ASOs was confirmed to have robust TMEM106B knockdown activity in RPE cells. Multiple ASOs, namely CMP ID NOs: 29_3, 15, and 26, reduced TMEM106B mRNA by about 80% at a concentration of 10 μM (FIG. 10A). ASO CMP ID NO: 14 reduced TMEM06b mRNA by about 50% at a concentration of 10 μM. ASO CMP ID NO: 10_13 showed almost no TMEM106B reduction and was, therefore, used as an ASO negative control (FIG. 10A). An increase in the ASO concentration needed to elicit the same level of knockdown was observed in human RPE cells relative to that of human iPSC-derived neurons. This is likely due to the continued proliferation of RPE cells during the five days of ASO treatment, thereby requiring higher ASO concentrations to elicit TMEM106B knockdown.

### Example 4: Caspase Assay to Determine Safety of TMEM106B-Targeting Antisense Oligonucleotides

To measure the in vitro toxicity profiles of ASOs described herein, a caspase assay was performed to measure apoptosis in treated cells. NIH 3T3-L1 cells (ATCC cat. CL-173) were transfected with 100 nM of an ASO using Lipofectamine2000 (Thermo Fisher 11668-019). Caspase 3 and Caspase 7 activation was measured after 24 hours as a proxy for apoptosis following the protocol described below, which was adapted from Dieckmann et al., Mol. Ther. Nucl. Acids 10:45-54 (2018). The experiment was performed in triplicate, with each replicate in a 96-well plate format. 24 hours before transfection, the cells were trypsinized and seeded in 96-well plates (ViewPlate-96, PerkinElmer, cat 6005181) at 4000 cells/well in 100 μL of Dulbecco's Modified Eagle Medium (DMEM) growth medium (Sigma, Cat. No. D0819), 10% fetal bovine serum (Sigma, Cat. No. F7524), 1 mM sodium pyruvate (Sigma Cat. No. S8636), and 0.025 mg/mL Gentamicin (Sigma, Cat. No. G1397) at 37° C. with 5% CO₂.

The ASOs were diluted with Dulbecco's Phosphate-Buffered Saline (DPBS) (Thermo Fisher Scientific, Cat. No. 14190250) to a final concentration of 5 μM. Negative controls were also prepared with 0 μM concentration of oligonucleotide. 9.6 μL of diluted ASO was mixed gently with 110.4 μL of Opti-MEM (Thermo Fisher Scientific, Cat. No. 31985047). 30 μL of LOM solution, which includes Lipofectamine™ 2000 (Thermo Fisher Scientific, Cat. No. 11668019) mixed with Opti-MEM (Thermo Fisher Scientific, Cat. No. 31985047) in a ratio of 1:99 (v/v), was used immediately after preparation, was added to each well of an empty 96-well plate. 30 μL of Opti-MEM diluted oligonucleotides was added to the LOM mix and followed by a 20-minute incubation. The media was removed from the NIH 3T3-L1 cell culture and 50% of culture medium, including of DMEM, 20% fetal bovine serum, and 1 mM sodium pyruvate, and 50 μL of the LNA oligonucleotide in LOM solution was added.

Next, 24 hours after adding the ASO to the cells, 100 μL of the Caspase-Glo® 3/7 reagent (Promega, Cat. No. G8093), prepared as according to the manufacturer's protocol, was added to the cells. The plates were shaken at 500 rpm for 30 seconds and incubated for one hour at room temperature and followed by blocking the back of the plates with BackSeal (PerkinElmer, Cat. No. 6005199). The luminescence was then measured using an EnSight Multimode Plate Reader (PerkinElmer, Cat. No. HH34000000).

The median of raw measurements for wells flagged as “Neutral” (no oligonucleotide) was calculated. Subsequently, each measurement within that plate was normalized by the median. The median of all “Neutral” and “Positive” wells (Positive=known cytotoxic oligonucleotides) was calculated separately within the same plate. Each well was scaled as: ((value−median (neutral))/(median (positive)−median (neutral))*100. Using the values calculated at the previous step, the median across the 3 replicates (3 plates) was calculated and reported for each well. The results of the Caspase assay are summarized in Table 8.

TABLE 8
Summary of caspase assay results

| | Caspase 3/7 |
|---|---|
| | activation (% of |
| CMP ID NO | positive control) |
| 29_5 | 6.36 |
| 10_13 | −3.97 |
| 20 | 11.31 |
| 25_1 | −2 |
| 15 | 5.98 (5.08, 6.87) |
| 24_3 | 4.36 |
| 8_3 | 7.4 |
| 8_4 | 16.91 |
| 14 | 2.25 |
| 8_1 | −3.98 |
| 6 | 2.07 |
| 10_4 | 18.57 |
| 24_1 | 12.74 |
| 12 | 4.63 |
| 29_3 | 5.71 |
| 26 | 3.93 (1.44, 6.41) |

### Example 5: In Vivo Efficacy of Antisense Oligonucleotides Targeting Human TMEM106B mRNA

A select panel of ASO agents described in Example 1 were tested for their ability to reduce TMEM106B expression in mouse brain in vivo. Transgenic mice expressing the human TMEM106B gene were generated via bacterial artificial chromosome (BAC) technology. Male and female mice (23-32 g) were given 5 μL of ASO compounds formulated in 0.9% saline, delivered by freehand injection into the right lateral ventricle with stereotaxic coordinates of −0.5 AP, 1.0 ML, 2.5 DV. Under isoflurane anesthesia, an incision was made in the skin directly above the center of the skull. Mice received an intracerebroventricular injection directly through the bone, and the incision was closed with surgical glue. Animals recovered on a heating pad and were monitored until fully alert.

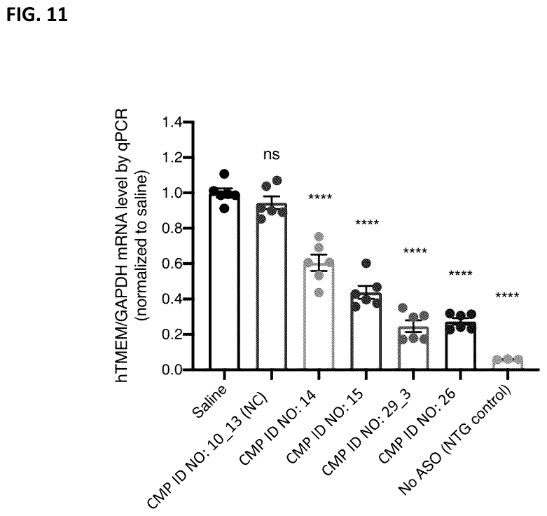

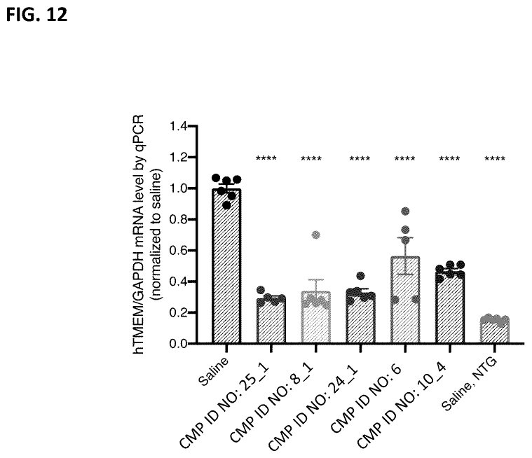

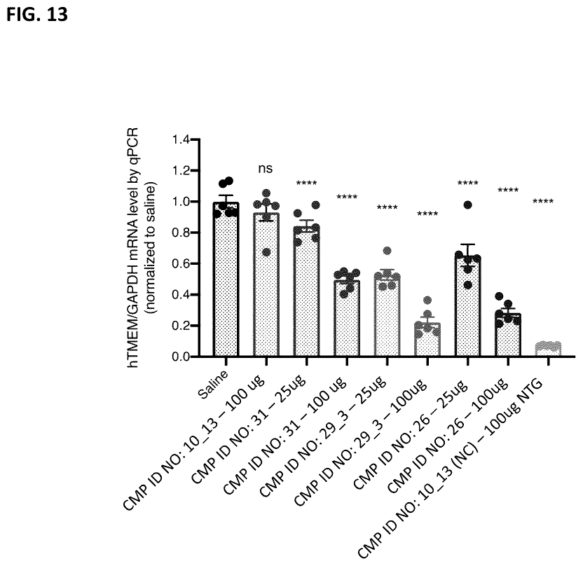

Four in vivo studies were conducted. Study 1 tested a single 100 μg dose of four ASOs of CMP ID NOs: 14, 15, 29_3, and 26, negative control ASO of CMP ID NO: 10_13, or saline (FIG. 11). Study 2 tested a single 100 μg dose of five ASOs of CMP ID NOs: 25_1, 8_1, 24_1, 6, and 10_4 and saline (FIG. 12). Study 3 tested two dose levels for each of CMP ID NO: 31, 29_3, and 26, negative control ASO of CMP ID NO: 10_13, or saline (FIG. 13). Study 4 tested two dose levels for CMP NO: 29_5. The same methods were applied in all four studies. Fourteen days after the injection, mice were sacrificed with isoflurane overdose followed by rapid decapitation, and the brain was placed in an ice-cold mouse brain slicer matrix (Stoelting) and sectioned into two (2 mm) coronal sections (starting at 0.26 mm from Bregma) weighing approximately 30-40 mg. The tissue was immersed in RNALater solution (ThermoFisher Scientific, cat. AM7021) and stored at 4° C. RNA was isolated using RNeasy Mini Kit (Qiagen, cat. 74116) using QIAcube robotic workstation (Qiagen). Briefly, a brain fragment was transferred from the RNALater solution to 1 mL RLT buffer in a 1.5 mL Eppendorf tube containing a 3 mm tungsten carbide bead (Qiagen, cat 69997). Tissue was lysed using TissueLyser II (Qiagen; 3 min at 30 Hz) and centrifuged for 3 minutes at 20,000 g. 550 μL of the lysate (no foam, no sediment) was transferred to a 2 mL Eppendorf tube and placed in QIAcube pre-filled with all the required reagents. RNA was purified according to a program “RNeasy Mini-Animal tissues and cells-Large samples (version 2)” with elution with 50 μL of H₂O. The RNA concentration was measured using NanoDrop 8000 (ThermoFisher Scientific) spectrophotometer and the concentration was adjusted with H₂O to 2 ng/μL.

Expression levels of TMEM106B mRNA on sections from the right hemisphere were measured as described in Example 1, using TMEM106B Hs00998849_ml (Thermo Fisher Scientific, cat. 4351370) as probe and a GAPDH probe (cat. 4351309, Applied Biosystems) for normalization. The data was analyzed using the QuantStudio™ Real_time PCR Software, and the readouts of the duplicates were averaged. Statistical analysis of TMEM106B mRNA reduction was analyzed using a one-way ANOVA in Graphpad Prism 5.0.

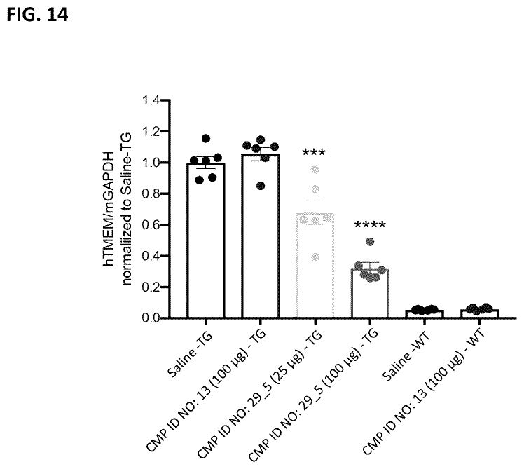

All tested TMEM106B-targeting oligonucleotides reduced TMEM106B mRNA levels two weeks after a single injection (FIGS. 11-14 and in Table 9, below). Data are shown as percent of average control samples (saline-treated mice; the lower the value the larger the inhibition). In Study 1, all four ASOs tested, namely CMP ID NOs: 14 (60% remaining, 15 (44% remaining), 29_3 (25% remaining), and 26 (27% remaining) produced significant reduction of TMEM106B compared to saline-treated mice ((F (6,32)=104.0, p<0.00001 followed by Dunnett's post-hoc test). The negative control ASO (CMP ID NO: 10_13) had no effect (95% remaining) on TMEM106B levels. In Study 2, all five ASOs significantly reduced TMEM106B transcript compared to saline-treated mice (FIG. 12; (F (6,33)=30.19, p<0.0001 followed by Dunnett's post-hoc test). Compound 25_1 is 29%, 8_1 is 34%, 24_1 is 33%, 6 is 56%, 10_4 is 47% remaining. In Study 3, all ASOs produced significant reduction of TMEM106B mRNA (F (8,45)=64.57, p<0.0001 followed by Dunnett's post-hoc test). ASOs of CMP ID NOs: 29_3 and 26 produced greater knockdown at 25 and 100 ug doses compared to CMP ID NO: 31 at the same dose levels (FIG. 13), indicating a 4 fold greater in vivo potency of CMP ID NOs: 29_3 and 26. (% KD for study 3: negative control ASO (100 ug) was not significantly different from saline-treated mice (93% remaining), CMP ID NO: 31 (25 ug) is 94% and (100 ug) is 50%, CMP ID NO: 29_3 (25 ug) is 53% and (100 ug) is 22%, CMP ID NO: 26 (25 ug) is 65% and (100 ug) is 28% remaining. Minimal side effects were observed in mice treated with ASOs described herein (FIG. 14). In Study 4, the negative control ASO CMPD ID NO: 13 had no effect on TMEM106B mRNA, while CMPD ID NO: 29_5 significantly reduced mRNA with 68% remaining at 25 ug (p<0.001) and 32% remaining at 100 ug (p<0.0001) (F (3,20)=41.4, p<0.0001 followed by Dunnett's post-hoc). As expected. non-transgenic controls expressed no human TMEM106b mRNA.

TABLE 9
In vivo knockdown of TMEM106B using antisense
oligonucleotides of the disclosure

| | In vivo TMEM106B RNA |
|---|---|
| CMP ID NO | remaining (100 μg icv) |
| 14 | 60% |
| 26 | 27% |
| 15 | 44% |
| 10_4 | 47% |
| 8_1 | 34% |
| 29_3 | 22% |
| 25_1 | 29% |
| 24_1 | 33% |
| 6 | 56% |
| 31 | 50% |
| 13 | 100% |
| 10_13 | 95% |
| 29_5 | 32% |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. An antisense oligonucleotide, wherein the antisense oligonucleotide is a compound selected from the group consisting of:
(SEQ ID NO: 19; CMP ID NO: 26)
TTCttctttaaatcCATC;
(SEQ ID NO: 20; CMP ID NO: 8_3)
ATaAgtgttcattcTCC;
(SEQ ID NO: 21; CMP ID NO: 8_4)
ATAAgTgttcattctCC;
(SEQ ID NO: 22; CMP ID NO: 12)
CATActattcttatTTCA;
(SEQ ID NO: 23; CMP ID NO: 24_3)
TTCAtactattcttatTtCA;
(SEQ ID NO: 24; CMP ID NO: 24_1)
TTCATactattcttatTTCA;
(SEQ ID NO: 25; CMP ID NO: 29_5)
TTtCAtActattcttaTTTC;
(SEQ ID NO: 26; CMP ID NO: 8_1)
ATAagtgttcattcTCC;
(SEQ ID NO: 27; CMP ID NO: 10_4)
ATtAtgtttatcaCCAa;
(SEQ ID NO: 28; CMP ID NO: 6)
AACagtctttttacTCC;
(SEQ ID NO: 29; CMP ID NO: 29_3)
TTTCatactattcttaTTTC;
(SEQ ID NO: 30; CMP ID NO: 14)
GACAtatacactcaAATA;
and
(SEQ ID NO: 31; CMP ID NO: 15)
GTtTatcaccaaaaTTAT,
wherein non-italicized capital letters are beta-D-oxy LNA nucleosides, italicized capital letters are 2′-O-methyl nucleosides, lowercase letters are DNA nucleosides, all LNA C are 5-methylcytosine, and all internucleoside linkages are phosphorothioate internucleoside linkages.

2. The antisense oligonucleotide of claim 1, wherein the antisense oligonucleotide is CMP ID NO: 26.

3. The antisense oligonucleotide of claim 1, wherein the antisense oligonucleotide is CMP ID NO: 8_3.

4. The antisense oligonucleotide of claim 1, wherein the antisense oligonucleotide is CMP ID NO: 8_4.

5. The antisense oligonucleotide of claim 1, wherein the antisense oligonucleotide is CMP ID NO: 12.

6. The antisense oligonucleotide of claim 1, wherein the antisense oligonucleotide is CMP ID NO: 24_3.

7. The antisense oligonucleotide of claim 1, wherein the antisense oligonucleotide is CMP ID NO: 24_1.

8. The antisense oligonucleotide of claim 1, wherein the antisense oligonucleotide is CMP ID NO: 29_5.

9. The antisense oligonucleotide of claim 1, wherein the antisense oligonucleotide is CMP ID NO: 8_1.

10. The antisense oligonucleotide of claim 1, wherein the antisense oligonucleotide is CMP ID NO: 10_4.

11. The antisense oligonucleotide of claim 1, wherein the antisense oligonucleotide is CMP ID NO: 6.

12. The antisense oligonucleotide of claim 1, wherein the antisense oligonucleotide is CMP ID NO: 29_3.

13. The antisense oligonucleotide of claim 1, wherein the antisense oligonucleotide is CMP ID NO: 14.

14. The antisense oligonucleotide of claim 1, wherein the antisense oligonucleotide is CMP ID NO: 15.

15. The antisense oligonucleotide of any one of claims 1-14, wherein the TMEM106B target nucleic acid is a mammalian TMEM106B target nucleic acid.

16. The antisense oligonucleotide of claim 15, wherein the mammalian TMEM106B target nucleic acid is a human TMEM106B target nucleic acid.

17. A conjugate comprising the antisense oligonucleotide of any one of claims 1-16, and at least one conjugate moiety covalently attached to said antisense oligonucleotide.

18. A pharmaceutically acceptable salt of the antisense oligonucleotide of any one of claims 1-16.

19. A pharmaceutically acceptable salt of the conjugate of claim 18.

20. A pharmaceutical composition comprising the antisense oligonucleotide of any one of claims 1-16 and a pharmaceutically acceptable diluent, solvent, carrier, salt or adjuvant.

21. A pharmaceutical composition comprising the conjugate of claim 17 and a pharmaceutically acceptable diluent, solvent, carrier, salt or adjuvant.

22. An in vivo or in vitro method for modulating TMEM106B expression in a target cell which is expressing TMEM106B, said method comprising administering the pharmaceutical composition of claim 20 or 21 in an effective amount to said cell.

23. A method for treating or preventing a disease comprising administering a therapeutically or prophylactically effective amount of the pharmaceutical composition of claim 20 or 21 to a subject suffering from or susceptible to the disease.

24. A pharmaceutical composition of claim 20 or 21 for use in the treatment or prevention of a disease.
